(19) United States
(12) Patent Application Publication
(10) Pub. No.: US 2019/0056304 A1
(43) Pub. Date: Feb. 21, 2019

(54) METHOD OF IMAGING BLOOD CELLS

(71) Applicant: Abbott Point of Care Inc., Princeton, NJ (US)

(72) Inventor: Sergey Gershtein, Skillman, NJ (US)

(73) Assignee: Abbott Point of Care Inc., Princeton, NJ (US)

(21) Appl. No.: 16/104,254

(22) Filed: Aug. 17, 2018

Related U.S. Application Data
(60) Provisional application No. 62/546,713, filed on Aug. 17, 2017.
(60) Provisional application No. 62/647,421, filed on Mar. 23, 2018.

(51) Int. Cl. G01N 15/14; G01N 33/80

(52) U.S. Cl. CPC G01N 15/1429; G01N 2015/0065; G01N 15/1404; G01N 33/80

(57) ABSTRACT

This present invention relates generally to devices, systems, and methods for performing bioimaging at the microscopic scale and, more particularly, to devices and systems including a disposable testing device configured to perform bioimaging at the microscopic scale, and methods of performing the bioimaging using the disposable testing device. In some aspects, a method is provided for performing a differential blood cell count. The method including moving a blood sample into a sample testing conduit having a first wall formed from at least a portion of an imager chip, a second wall formed from a transparent material layer, and a plurality of spacer elements. The method further including driving a light emitter to project light through the chamber, recording an output signal of at least one of absorbance and fluorescence, and converting the output signal to a number count or percentage for each type of cell in the blood sample.

### CROSS-REFERENCE TO RELATED APPLICATIONS

This application claims priority to U.S. Provisional Application No. 62/546,713 filed on Aug. 17, 2017 and U.S. Provisional Application No. 62/647,421 filed on Mar. 23, 2018, the entireties of which are incorporated herein by reference.

### FIELD OF THE INVENTION

This present invention relates generally to devices, systems, and methods for performing bioimaging at the microscopic scale and, more particularly, to devices and systems including a disposable testing device configured to perform bioimaging at the microscopic scale, and methods of performing the bioimaging using the disposable testing device.

### BACKGROUND OF THE INVENTION

Point-of-care (POC) sample analysis systems are typically based on one or more re-usable hand-held analyzers (i.e., instruments or reading apparatus) that perform sample tests using a single-use disposable testing device, e.g., a cartridge or strip that contains analytical elements, e.g., electrodes or optics for sensing analytes such as pH, oxygen and glucose, as well as various types of proteins, enzymes and blood cells. The disposable testing device may include fluidic elements (e.g., conduits for receiving and delivering the sample to sensing electrodes or optics), calibrant elements (e.g., aqueous fluids for standardizing the electrodes and optics with a known concentration of analyte), and dyes with known extinction coefficients for standardizing optics. The instrument or reading apparatus may contain electrical circuitry and other components for operating the electrodes or optics, making measurements, and performing computations. The instrument or reading apparatus may also have the ability to display results and communicate those results to laboratory and hospital information systems (LIS and HIS, respectively), for example, via a computer workstation or other data management system. Communication between the instrument or reading apparatus and a workstation, and between the workstation and a LIS or HIS, may be via, for example, an infrared link, a wired connection, wireless communication, or any other form of data communication that is capable of transmitting and receiving electrical information, or any combination thereof. A notable point-of-care system (The i-STAT® System, Abbott Point of Care Inc., Princeton, N.J.) is disclosed in U.S. Pat. No. 5,096,669, which is incorporated herein by reference in its entirety. The i-STAT® System comprises one or more disposable testing devices, operating in conjunction with a hand-held analyzer, for performing a variety of measurements on biological specimens such as blood.

One benefit of point-of-care sample testing systems is the elimination of the time-consuming need to send a sample to a central laboratory for testing. Point-of-care sample testing systems allow a nurse or doctor (user or operator), at the bedside of a patient, to obtain a reliable quantitative analytical result, comparable in quality to that which would be obtained in a laboratory. In operation, the nurse selects a testing device with the required panel of tests, draws a biological sample from the patient, dispenses the biological sample into the testing device, optionally seals the testing device, and inserts the testing device into the instrument or reading apparatus. While the particular order in which the steps occur may vary between different point-of-care systems and providers, the intent of providing rapid sample test results close to the location of the patient remains the same. The instrument or reading apparatus then performs a test cycle, i.e., all the other analytical steps required to perform the tests. Such simplicity gives the doctor quicker insight into a patient's physiological status and, by reducing the turnaround time for diagnosis or monitoring, enables a quicker decision by the doctor on the appropriate treatment, thus enhancing the likelihood of a successful patient outcome.

As discussed herein, point-of-care sample testing systems typically include an instrument or analyzer configured to perform sample tests using single-use disposable testing device for the determination of analytes in biological samples. The type of sample tests performed may vary and can be implemented using one or more disposable testing devices including, for example, a qualitative or semi-quantitative testing device (e.g., lateral flow or microarray assays), a quantitative testing device (e.g., an electrochemical assay), or a combined qualitative or semi-quantitative testing device and a quantitative testing device (e.g., a testing device with both lateral flow or microarray assays and an electrochemical assay). In order to perform the sample tests, the instrument or analyzer includes an optical sensor configured to process a signal from the qualitative or semi-quantitative testing device and/or an electrical connector configured to process a signal from the quantitative testing device (see, e.g., U.S. Pat. No. 9,194,859, which is incorporated herein by reference in its entirety). In particular, the optical sensor includes an optical imager configured to image an assay of an optical test cartridge. The assay is a qualitative or semi-quantitative lateral flow test or microarray test (e.g., a one or more lateral flow test strips or microarrays disposed in a conduit of the optical test cartridge). The optical sensor further includes a processor configured to process a signal generated by the optical imager to display a qualitative or semi-quantitative test result.

However, a problem associated with these conventional instruments or analyzer is that they are incapable of performing bioimaging at the microscopic scale, which is important for hematology assays (e.g., cell counting). Moreover, the optical imager is a set type hardwired to a non-disposable instrument or analyzer, and thus there is limited flexibility in performing multiple types of assays (e.g., hematology and immunoassays) within the testing devices without hardware changes. The limitations imposed by having the optical sensor within the non-disposable instrument or analyzer combined with the hardwired design adversely affects the capability of the instruments or analyzer to perform bioimaging at the microscopic scale and multiple tests or measurements without hardware changes.

Conventionally, bioimaging at the microscopic scale (e.g., performing hematological assays such as cell counting and differentials) has been performed using lenses, such as with optical microscopy which utilizes objective lenses for magnification of blood cells. Recently, however, imaging without lenses has matured as a modality competitive with conventional lens-based microscopy. In lensless microscopy, a diffraction pattern resulting from an object (based on, e.g., scattering or fluorescence) is recorded directly on a digital image sensor array without being optically imaged or magnified by any lens elements. The recorded diffraction pattern is then computationally reconstructed to form an “image” of the object(s). The recent maturation of lensless microscopy was made possible largely by the mass production of inexpensive digital image sensors with small pixel size and high pixel counts, along with improvements in computing power and reconstruction algorithms used to process the captured diffraction patterns. Compared with conventional lens-based microscopy, lensless approaches impart several key advantages including: a large space-bandwidth product (large field of view and high resolution simultaneously), high resolution, cost-effectiveness, and portability.

In order to take advantage of lensless microscopy, an alternative type of assay system has been developed using digital image sensors and specially designed chambers that permit the enumeration of particulate matter (e.g., blood cells) within the sample. For example, U.S. Pat. No. 7,850,916, which is incorporated herein by reference in its entirety, describes a chamber for the enumeration of particulate matter (e.g., blood cells) that includes a first planar member that is flexible, a second planar member, and at least three separators. The characterization of the blood cells (e.g., a white blood cell differential count) within the chamber may be performed by classifying each individual blood cell as it is encountered using either traditional image-processing methods or by the techniques described in U.S. Pat. Nos. 5,321,975, 6,235,536, 6,350,613, 8,797,527, and 9,041,790, US Patent Publication Nos. 20130169948 and 20120034647, and Aydogan Ozcan and Euan McLeod, Lensless Imaging and Sensing, Annu. Rev. Biomed. Eng. 2016. 18:77-102, doi:10.1146/annurev-bioeng-092515-010849, all of which are incorporated herein by reference in their entirety.

However, a problem associated with these lensless microscopy assay systems is that typically one or more components of the system such as the digital image sensor array, the light source, the imaging chamber, and the image processing software/hardware are not designed appropriately to lend themselves to point-of-care utilization. Specifically, these conventional lensless microscopy testing systems lack portability and disposability which are aspects normally associate with point-of-care testing devices. Therefore, there exists within the field of bioimaging at the microscopic scale, and in particular for applications in which cells and analytes are to be determined within biological samples such as blood, a need for devices that can rapidly and simply determine the presence, count, identity, and/or concentration of cells and analytes at a patient's point-of-care, and can be performed by less highly trained staff than is possible for conventional laboratory-based testing. It would, for example, be of benefit in the diagnosis and treatment of critical medical conditions for the attending physician or nurse to be able to obtain optical assay results such as cell counts and differentials at the patient's bedside without delay.

### SUMMARY OF THE INVENTION

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. In various embodiments, a method is provided for performing a differential blood cell count including: providing a test cartridge including a sample entry port, a sample testing conduit fluidically connected to the sample entry port, and an imager chip including an array of pixels. The method also includes providing an analyzer including a processor and display; mating the test cartridge with the analyzer. The method also includes introducing a blood sample into the sample entry port before or after the mating the test cartridge with the analyzer. The method also includes dissolving a dry reagent into the blood sample to generate an amended blood sample. The method also includes moving the amended blood sample into the sample testing conduit, where the sample testing conduit includes a first wall formed from at least a portion of an imager chip, a second wall formed from a transparent material layer, and a plurality of spacer elements having an average spacer height and disposed between the first wall and the second wall, and where the average spacer height defines an average chamber height of a chamber between the portion of the imager chip and the transparent material layer. The method also includes driving a light emitter to project light through the chamber and the amended blood sample. The method also includes recording an output signal of at least one of absorbance and fluorescence at the array of pixels based on the light received from the chamber and the amended blood sample. The method also includes converting the output signal using the processor to a number count or percentage for each type of cell in the blood sample. The method also includes displaying the number count or percentage for each type of cell in the blood sample on the display. Other embodiments of this aspect include corresponding computer systems, apparatus, and computer programs recorded on one or more computer storage devices, each configured to perform the one or more actions of the methods.

Implementations of the method may include one or more of the following features. The method further including: unmating the test cartridge from the analyzer. The method may also include disposing of the test cartridge. The method where the dry reagent includes acridine orange or an anticoagulant. The method where the dry reagent binds to nucleic dna in the blood cells. The method where the mating includes inserting the test cartridge into a port of the analyzer. The method where the analyzer further includes a multi-terminal connector, the test cartridge further includes a plurality of discrete connector contacts, the imager chip is electrically connected to at least one of the plurality of discrete connector contacts, and the inserting the test cartridge into the port of the analyzer places the multi-terminal connector in electrical contact with the plurality of discrete connector contacts. The method where the processor is electrically connected to the light emitter, the processor is electrically connected to the imager chip via the at least one of the plurality of discrete connector contacts and the multi-terminal connector, the light emitter is driven via the processor to project the light, and the imager chip is controlled via the processor to record the output signal. The method the analyzer further includes a pump actuator, the test cartridge further includes a pump, the inserting the test cartridge into the port of the analyzer places the pump actuator aligned with the pump, and moving the amended blood sample into the sample testing conduit includes driving the pump actuator to actuate the pump and displace the amended blood sample into the sample testing conduit. Implementations of the described techniques may include hardware, a method or process, or computer software on a computer-accessible medium.

In various embodiments, a system is provided including: one or more processors. The system also includes memory coupled to the one or more processors, the memory encoded with a set of instructions configured to perform a process including: The system also includes receiving an operating state signal from a test cartridge indicative of a type of cartridge inserted into an analyzer. The system also includes determining that the type of cartridge is the test cartridge having a contact connected to an imager chip configured to image blood cells in a blood sample. The system also includes driving a pump actuator to actuate a pump on the test cartridge and move the blood sample from a sample receiving chamber into a sample testing conduit, where the sample testing conduit includes a first wall formed from at least a portion of the imager chip, a second wall formed from a transparent material layer, and a plurality of spacer elements disposed between the first wall and the second wall. The system also includes driving a light emitter to project light through the sample testing conduit and the blood sample. The system also includes recording an output signal of at least one of absorbance and fluorescence at an array of pixels of the imager chip based on the light received from the sample testing conduit and the blood sample. The system also includes converting the output signal to a number count or percentage for each type of cell in the blood sample.

Implementations of the system may include one or more of the following features. The system where the method further includes driving the pump actuator to actuate the pump on the test cartridge and move the blood sample into contact with a dry reagent. The system where the dry reagent includes acridine orange or an anticoagulant. The system where the dry reagent binds to nucleic dna in the blood cells. The system where the dry regent is disposed in the sample receiving chamber. The system where the dry regent is disposed in the sample testing conduit. The system further including displaying the number count or percentage for each type of cell in the blood sample on a display. The system where the light emitter projects the light through the transparent material layer, the sample testing conduit and the blood sample. The system where the light emitter is disposed in the test cartridge, and determining that the type of cartridge is the test cartridge having the contact connected to the imager chip and an another contact connected to the light emitter. The system where the plurality of spacer elements have a predetermined average spacer height that defines a predetermined average chamber height of a chamber between the portion of the imager chip and the transparent material layer. The system where at least one of the second wall and the plurality of spacer elements is deformable such that the second wall and the plurality of spacer elements are drawn toward each other by capillary force from the blood sample being moved into the sample testing conduit.

In various embodiments, a non-transitory machine readable storage medium is provided for storing instructions that, when executed by one or more processors of a computing system, cause the computing system to perform operations including: driving a pump actuator to actuate a pump on a test cartridge and move a blood sample from a sample receiving chamber into a sample testing conduit, where the sample testing conduit includes a first wall formed from at least a portion of an imager chip, a second wall formed from a transparent material layer, and a plurality of spacer elements disposed between the first wall and the second wall. The non-transitory machine readable storage medium also includes driving a light emitter to project light through the sample testing conduit and the blood sample. The non-transitory machine readable storage medium also includes recording an output signal of at least one of absorbance and fluorescence at an array of pixels of the imager chip based on the light received from the sample testing conduit and the blood sample. The non-transitory machine readable storage medium also includes converting the output signal to a number count or percentage for each type of cell in the blood sample.

Implementations of the non-transitory machine readable storage medium may include one or more of the following features. The non-transitory machine readable storage medium where the operations further include driving the pump actuator to actuate the pump on the test cartridge to split the blood sample into a first portion and a second portion, where the first portion of the blood sample is moved into the sample testing conduit. The non-transitory machine readable storage medium where the operations further include driving the pump actuator to actuate the pump on the test cartridge to move the second portion of the blood sample into an auxiliary conduit including an electrochemical sensor for detecting an analyte in the blood sample. The non-transitory machine readable storage medium where the operations further include recording an analyte signal from the electrochemical sensoheightr based on performance of an electrochemical analytical test in the auxiliary conduit, and determining a qualitative, semi-quantitative, or quantitative value proportional to an amount of the analyte in the blood sample based on the analyte signal. The non-transitory machine readable storage medium where the performing the electrochemical analytical test includes applying a potential to the electrochemical sensor with respect to a reference electrode, and measuring a current change across the blood sample that is proportional to the amount of the analyte within the blood sample, and where the analyte signal is recorded as indicative of the measured current change across the blood sample. The non-transitory machine readable storage medium where the operations further include receiving an operating state signal from the test cartridge indicative of a type of cartridge inserted into an analyzer, and determining that the type of cartridge is the test cartridge having a first contact connected to the imager chip and a second contact connected to the electrochemical sensor. The non-transitory machine readable storage medium where the plurality of spacer elements have a predetermined average spacer height that defines a predetermined average chamber height of a chamber between the portion of the imager chip and the transparent material layer. The non-transitory machine readable storage medium where at least one of the second wall and the plurality of spacer elements is deformable such that the second wall and the plurality of spacer elements are drawn toward each other by capillary force from the blood sample being moved into the sample testing conduit.

### BRIEF DESCRIPTION OF THE DRAWINGS:

The present invention will be better understood in view of the following non-limiting figures.

FIG. 1 shows a disposable testing device and instrument in accordance with various embodiments;

FIG. 2 shows an illustrative architecture of a computing system implemented in accordance with various embodiments;

FIGS. 3 and 4A-4R show a testing device or cartridge in accordance with various embodiments;

FIG. 5 shows an imaging device in accordance with various embodiments;

FIG. 6 shows a cross-section of an imaging chamber in accordance with various embodiments;

FIG. 7 shows a top planar view of an imaging chamber in accordance with various embodiments;

FIG. 8 shows an imaging chamber comprising a first planar member, a second planar member, and a plurality of spacer elements disposed between the first planar member and the second planar member in accordance with various embodiments;

FIG. 9 illustrates the plurality of spacer elements of the imaging chamber may be formed from a material that has greater flexibility than the first planar member and the second planar member in accordance with various embodiments;

FIG. 10 illustrates the second planar member of the imaging chamber may be formed from a material that has greater flexibility than the plurality of spacer elements and the first planar member in accordance with various embodiments;

FIGS. 11A-11I show an imaging chamber comprising a first planar member, a second planar member, and a plurality of wells disposed between the first planar member and the second planar member in accordance with various embodiments;

FIG. 12 shows wafer-level micro-fabrication of an imager chip in accordance with various embodiments;

FIG. 13 shows wafer-level micro-fabrication of an electrochemical chip in accordance with various embodiments;

FIG. 14 shows a sensor chip configuration in accordance with various embodiments;

FIG. 15 shows an alternative sensor chip configuration in accordance with various embodiments; and

FIGS. 16-22 show exemplary flowcharts for performing process steps in accordance with various embodiments.

### DETAILED DESCRIPTION OF THE INVENTION

### Introduction

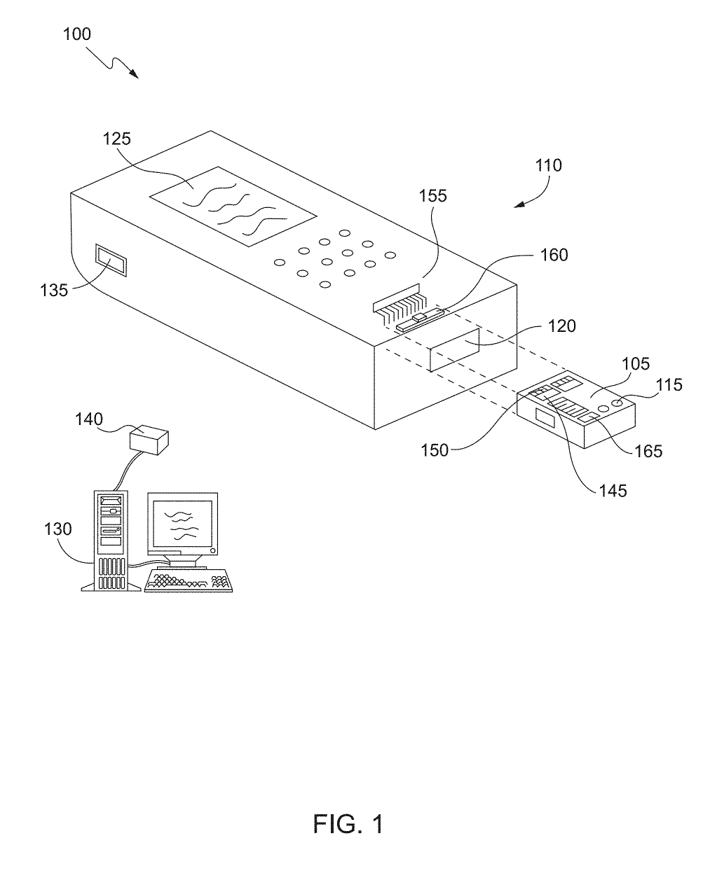

Various embodiments of the present invention are directed to devices, systems, and methods for performing optical and optionally electrochemical assays. For example, FIG. 1 shows an exemplary system 100 that may comprise a self-contained disposable testing device or cartridge 105 and an instrument or reading apparatus 110 (e.g., an analyzer) that is portable or stationary and battery powered or line powered. In some embodiments, the testing device 105 is a single-use device configured to be disposable after the single-use. A fluid sample (e.g., whole blood) to be measured is drawn into a sample receiving chamber via a sample entry orifice 115 in the testing device 105, and the testing device 105 may be inserted into the analyzer 110 through a port 120. The analyzer 110 may comprise a processor configured to perform processes including but not limited to: driving a light emitter, optical sensor, a pump, and/or electrochemical sensor; obtaining an output signal of at least one of absorbance and fluorescence (optical), current (amperometric), potential or charge accumulation (potentiometric), a conductive property of a medium between electrodes (conductometric), and impedance (both resistance and reactance); and converting the output signal to: (i) a number count or percentage for each type of cell in a blood sample, or (ii) a value indicative of a reaction of the biological sample with at least one assay bead. Measurements and determinations performed by the analyzer 110 (for example, (i) the number count or percentage for each type of cell in the blood sample, or (ii) the value indicative of the reaction) may be output to a display 125 or other output device, such as a printer or data management system 130 via a port 135 on the analyzer 110 to a computer port 140. Transmission can be via wired or wireless communication such as a telephone network, Internet connection, Wi-Fi, Bluetooth link, infrared and the like. The sensor(s) 145 (e.g., an optical sensor) in the testing device 105 include a plurality of discrete connector contacts 150 that make electrical contact with the analyzer 110 via a multi-terminal connector 155 when the testing device 105 is inserted into the port 140. For example, the multi-terminal connector 155 may be of the design disclosed in U.S. Pat. No. 4,954,087, which is incorporated herein by reference in its entirety. The analyzer 110 may further comprises a pump actuator 160 and the testing device 105 may further comprise a pump 165. In some embodiments, the inserting the testing device 105 into the port 120 of the analyzer 105 places the pump actuator 160 aligned with the pump 165, and the fluid sample may be moved into a sample testing conduit of the testing device 105 by driving the pump actuator 160 to actuate the pump 165 and displace the fluid sample into the sample testing conduit. In certain embodiments, the analyzer 110 is further configured to perform a method for automatic fluid flow compensation in the testing device 105, as disclosed in U.S. Pat. No. 5,821,399, which is also incorporated herein by reference in its entirety.

To specifically address problems associated with conventional instruments or reading apparatus, some embodiments described herein are directed to devices and systems including a disposable testing device configured to perform bioimaging at the microscopic scale, and methods of performing the bioimaging using the disposable testing device. In one embodiment, a test device for imaging blood cells in a blood sample is provided that includes: a sample entry port for receiving the blood sample; a sample receiving chamber fluidically connected to the sample entry port; a sample testing conduit fluidically connected to the sample receiving chamber, the sample testing conduit comprising: (i) a planar member, (ii) a transparent planar member, and (iii) a plurality of spacer elements having an average spacer height and disposed between the planar member and the transparent planar member to form a chamber having an average chamber height extending between the planar member and the transparent planar member; and an imager chip forming at least a portion of the planar member.

In another embodiments, a system for imaging blood cells in a blood sample is provided for that comprises: an analyzer comprising: a port, a multi-terminal connector, a processor connected to the multi-terminal connector, and memory coupled to the processor; and a test cartridge comprising: a plurality of connector contacts, a sample receiving chamber configured to receive the blood sample, a sample testing conduit fluidically connected to the sample receiving chamber, the sample testing conduit comprising: (i) a first wall, (ii) a second wall, and (iii) a plurality of spacer elements having a predetermined average spacer height and disposed between the first wall and the second wall to form a chamber having a predetermined average chamber height extending between the first wall and the second wall, and an analyte assay region comprising: a portion of the chamber and an imager chip, wherein the imager chip is electrically connected to at least one of the plurality of connector contacts, and at least a portion of the imager chip forms a portion of the first wall of the sample receiving chamber. The test cartridge is insertable into the port such that the multi-terminal connector is in electrical contact with the plurality of connector contacts.

In another embodiment, a method is provided for performing a differential blood cell count comprising: providing a test cartridge comprising a sample entry port, a sample testing conduit fluidically connected to the sample entry port, and an imager chip comprising an array of pixels; providing an analyzer comprising a processor and display; mating the test cartridge with the analyzer; introducing a blood sample into the sample entry port before or after the mating the test cartridge with the analyzer; dissolving a dry reagent into the blood sample to generate an amended blood sample; moving the amended blood sample into the sample testing conduit, wherein the sample testing conduit comprises a first wall formed from at least a portion of an imager chip, a second wall formed from a transparent material layer, and a plurality of spacer elements having an average spacer height and disposed between the first wall and the second wall, and wherein the average spacer height defines an average chamber height of a chamber between the portion of the imager chip and the transparent material layer; driving a light emitter to project light through the chamber and the amended blood sample; recording an output signal of at least one of absorbance and fluorescence at the array of pixels based on the light received from the chamber and the amended blood sample; converting the output signal using the processor to a number count or percentage for each type of cell in the blood sample; and displaying the number count or percentage for each type of cell in the blood sample on the display.

In another embodiment, a test device is provided for imaging assay beads, comprising: a sample entry port for receiving a biological sample; a sample receiving chamber fluidically connected to the sample entry port; and a sample testing conduit fluidically connected to the sample receiving chamber, the sample testing conduit comprising: (i) a first planar member, (ii) a second planar member, and (iii) a plurality of wells having a predetermined average well height and disposed between the first planar member and the second planar member. The second planar member comprises an imager chip comprising an array of pixels; and each of the plurality of wells is aligned vertically with one or more of the pixels in the array of pixels.

In another embodiment, a system is provided for imaging assay beads, comprising: an analyzer comprising: a port, a multi-terminal connector, a processor connected to the multi-terminal connector, and memory coupled to the processor; and a test cartridge comprising: a plurality of discrete connector contacts, a sample receiving chamber configured to receive a biological sample, a sample testing conduit fluidically connected to the sample receiving chamber, the sample testing conduit comprising: (i) a first wall, (ii) a second wall, and (iii) a plurality of wells having an average well height and disposed between the first wall and the second wall, and an analyte assay region comprising: a portion of the sample testing conduit and an imager chip, wherein the imager chip is electrically connected to at least one of the plurality of discrete connector contacts, and at least a portion of the imager chip forms a portion of the first wall of the sample receiving chamber. Each of the plurality of wells is aligned vertically with one or more pixels of the imager chip and each of the plurality of wells comprise at least one assay bead. The test cartridge is insertable into the port such that the multi-terminal connector is in electrical contact with the plurality of discrete connector contacts. The memory is encoded with a set of instructions configured to perform an analytical test on the biological sample, and to perform the analytical test, (i) the processor is electrically connected to a light emitter, (ii) the processor is electrically connected to the imager chip via at least one of the plurality of discrete connector contacts and the multi-terminal connector, (iii) the processor is configured to drive the light emitter to generate light projected into the portion of the sample testing conduit, (iv) the imager chip is configured to convert light received from the portion of the sample testing conduit to an output signal, and (v) the processor is configured to convert the output signal of the imager chip to a value indicative of a reaction of the biological sample with the at least one assay bead in each of the plurality of wells.

In another embodiment, a method is provided for imaging assay beads comprising: mating a test cartridge with an analyzer, wherein the test cartridge comprises a sample entry port, a sample testing conduit fluidically connected to the sample entry port, and an imager chip, and the analyzer comprises a processor and display; introducing a biological sample into the sample entry port before or after the mating the test cartridge with the analyzer; moving the biological sample into the sample testing conduit, wherein the sample testing conduit comprises a first wall formed from at least a portion of an imager chip, a second wall formed from a transparent material layer, and a plurality of wells having an average well height and disposed between the first wall and the second wall, and wherein each of the plurality of wells is aligned vertically with one or more pixels of the imager chip and at least a portion of the plurality of wells comprise at least one assay bead; driving a light emitter to project light through the plurality of wells; recording an output signal of at least one of absorbance and fluorescence at the pixels of the imager chip based on the light received from the plurality of wells; converting the output signal using the processor to a value indicative of a reaction of the biological sample with the at least one assay bead in each of the plurality of wells; and displaying the value on the display.

Advantageously, these approaches provide devices, systems, and methods with greater flexibility in testing device design including: (i) the combination of tests in any given testing device, (ii) the combination of tests on any given sensor chip, (iii) the position of sensors within the testing device, (iv) extending utility of the analyzers to perform various types of assays without hardware changes, and (v) increasing the point-of-care testing opportunities. In addition, these approaches may also reduce the number of different testing device bases (which accommodate the sensor chips) used to manufacture all the different testing devices for the various test. In addition, these approaches allow for bioimaging at the microscopic scale, and in particular for applications in which the count and/or identification of cells and optionally the concentration of analytes at patient point-of-care can be performed by less highly trained staff than is possible for conventional laboratory-based testing.

### System Environment

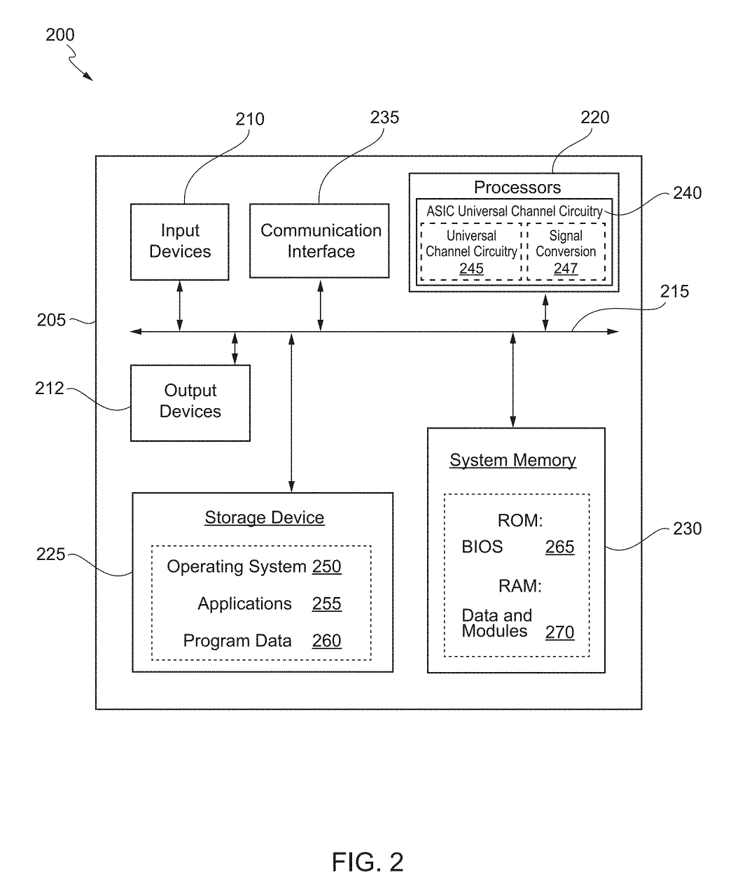

FIG. 2 is an illustrative architecture of a computing system 200 implemented in various embodiments. The computing system 200 is only one example of a suitable computing system and is not intended to suggest any limitation as to the scope of use or functionality of the various embodiments. Also, computing system 200 should not be interpreted as having any dependency or requirement relating to any one or combination of components illustrated in computing system 200.

As shown in FIG. 2, computing system 200 includes a computing device 205. The computing device 205 can be resident on a network infrastructure such as within a cloud environment, or may be a separate independent computing device (e.g., a computing device implemented within the environment of an analyzer such as analyzer 110 as described with respect to FIG. 1)). The computing device 205 may include one or more input devices 210, one or more output devices 212, a bus 215, processor 220, a storage device 225, a system memory (hardware device) 230, and a communication interface 235.

The one or more input devices 210 may include one or more mechanisms that permit an operator to input information to computing device 205, such as, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 212 may include one or more mechanisms that output information to an operator, such as, but not limited to, audio speakers, headphones, audio line-outs, visual displays, antennas, infrared ports, tactile feedback, printers, or combinations thereof. The bus 215 permits communication among the components of computing device 205. For example, bus 215 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 205.

The processor 220 may be one or more integrated circuits, printed circuits, controllers, microprocessors, or specialized dedicated processors that include processing circuitry operative to interpret and execute computer readable program instructions, such as program instructions for controlling the operation and performance of one or more of the various other components of computing device 205 for implementing the functionality, steps, and/or performance of the embodiments discussed herein. In certain embodiments, processor 220 interprets and executes the processes, steps, functions, and/or operations, which may be operatively implemented by the computer readable program instructions. For example, processor 220 can receive an operating state signal from a test cartridge indicative of a type of cartridge inserted into an analyzer; determine that the type of cartridge is the test cartridge having a contact connected to a sensor chip configured to image blood cells in a blood sample; drive a pump actuator to actuate a pump on the test cartridge and move the blood sample from a sample receiving chamber into a sample testing conduit, drive a light emitter to project light through the sample testing conduit and the blood sample, record an output signal of at least one of absorbance and fluorescence at an array of pixels of a sensor chip based on the light received from the sample testing conduit and the blood sample, and convert the output signal to a number count or percentage for each type of cell in the blood sample. In some embodiments, the information obtained or generated by the processor 220, e.g., type of test cartridge, number count or percentage for each type of cell, circuit configurations for channels, a tally for various operations, output current, look-up tables, potential to be applied, etc., can be stored in the storage device 225. In certain embodiments, the processor 220 comprises a thermal controller for controlling a temperature of the biological sample or specimen in a portion of a conduit.

In various embodiments, the processor 220 comprises an application-specific integrated circuit 240 that includes universal channel circuitry 245, and analog to digital signal converter 247. In other embodiments, the processor 220 is in communication with the application-specific integrated circuit 240 that includes the universal channel circuitry 245. The application-specific integrated circuit 240 is an integrated circuit (IC) customized for performing a number of functions including an analog to digital signal interface, current to voltage conversion, multiplexing, resistor selection, signal amplification, potential and conductance generation and/or measurement, and the performance of multiple types of assays. The universal channel circuitry 245 includes circuitry that can be implemented in conjunction with computer readable program instructions, data structures, program modules and other data to switch between various modes or configurations (e.g., a potentiometric mode, an amperometric mode, a conductance mode, an optical mode, etc.) and contribute to the performance of multiple types of assays.

The storage device 225 may include removable/non-removable, volatile/non-volatile computer readable media, such as, but not limited to, non-transitory machine readable storage medium such as magnetic and/or optical recording media and their corresponding drives. The drives and their associated computer readable media provide for storage of the computer readable program instructions, data structures, program modules and other data for operation of computing device 205. In various embodiments, storage device 225 stores operating system 250, application programs 255, and/or program data 260. In some embodiments, the application programs 255, and/or program data 260 may include a database, index, or table, and algorithms, for example, firmware or software for image analysis, data storage, illumination control, data display, sample analysis, and sample movement. In some embodiments, the computing system 200 implements algorithms, which provide the instructions for execution of processor 220, to enhance, detect, analyze, characterize, and measure images of cells and other specimens of interest and to display or transmit the result of these algorithms to a human operator and/or a second computer-based system, such as a personal computing device (e.g., a smartphone) or storage system including hospital record storage systems. In certain embodiments, the computing system 200 implements qualitative, semi-quantitative, or quantitative value algorithms that include components for determining a presence and/or amount of target analyte in a biological specimen or sample, a position determining algorithm for determining the location of a biological sample within a test device based on detected conductance, a cell count and differential algorithm, and a hematocrit determination algorithm for determining a hematocrit of a biological sample based on detected conductance across a biological sample, which provide the instructions for execution of processor 220.

The system memory 230 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 265 (BIOS) including the basic routines that help to transfer information between the various other components of computing device 205, such as during start-up, may be stored in the ROM. Additionally, data and/or program modules 270, such as at least a portion of operating system 250, application programs 255, and/or program data 260, that are accessible to and/or presently being operated on by processor 220, may be contained in the system memory 230.

The communication interface 235 may include any transceiver-like mechanism (e.g., a network interface, a network adapter, a modem, or combinations thereof) that enables computing device 205 to communicate with remote devices or systems, such as other analyzers, a hospital information system, a mobile device or other computing devices such as, for example, a server in a networked environment, e.g., cloud environment. For example, computing device 205 may be connected to remote devices or systems via one or more local area networks (LAN) and/or one or more wide area networks (WAN) using communication interface 235.

As discussed herein, computing system 200 may be configured to perform one or more analytical tests (e.g., a hematology assay). In particular, computing device 205 may perform tasks (e.g., process, steps, methods and/or functionality) in response to processor 220 executing program instructions contained in non-transitory machine readable storage medium, such as system memory 230. The program instructions may be read into system memory 230 from another computer readable medium (e.g., non-transitory machine readable storage medium), such as data storage device 225, or from another device via the communication interface 235 or server within or outside of a cloud environment. In some embodiments, hardwired circuitry of computing system 200 is used in place of or in combination with the program instructions to implement the tasks, e.g., steps, methods and/or functionality, consistent with the different aspects discussed herein. Thus, the steps, methods and/or functionality disclosed herein can be implemented in any combination of hardware circuitry and software.

### Testing Device or Cartridge

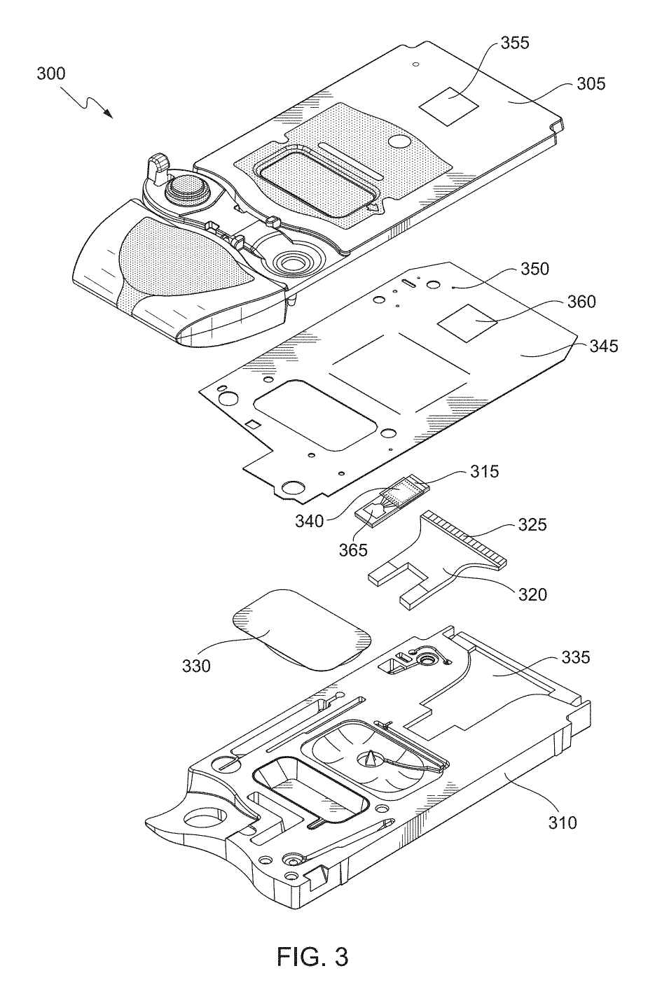

In one embodiment, as shown in FIG. 3, a testing device or cartridge 300 (e.g., testing device 105 as described with respect to FIG. 1) comprises a top portion 305 (e.g., a cover) and a bottom portion 310 (e.g., a base) in which are mounted at least one microfabricated sensor chip 315 disposed on a imager chip carrier 320 (e.g., a substrate) with electrical contacts 325 and optionally a pouch 330 containing a fluid, e.g., a calibrant fluid, a diluent fluid, a reagent, and/or a wash fluid. In some embodiments, the composition of the fluid in the pouch 330 is selected from the group consisting of water, calibrant fluid, reagent fluid, control fluid, wash fluid and combinations thereof. The sensor chip 315 and imager chip carrier 320 may be positioned in recessed region 335 and configured to generate electric signals based on, for example, light transmitted through an imaging chamber 340 of the sensor chip 315 and a biological specimen, e.g., a blood sample from a patient. A gasket 345 may be situated between the top portion 305 and the bottom portion 310 to bond them together, and to define and seal several cavities and conduits within the cartridge 300. The gasket 345 may cover substantially the entire area between the top portion 305 and the bottom portion 310 of the cartridge 300, as shown in FIG. 3, or may be localized over and between only predetermined structural features, e.g., the sensor chip 315 of the cartridge 300 (not shown). The gasket 345 may include apertures 350 to enable physical, fluidic and/or gaseous communication between structural features of the top portion 305 and the bottom portion 310. The gasket 345 may or may not have an adhesive surface, and may have an adhesive surface on both sides thereof, i.e., forming a double-sided adhesive layer. In some embodiments, in which a light emitter is provided by an external element such as the analyzer (e.g., the reading apparatus 110 as discussed with respect to FIG. 1), the top portion 305 and the gasket 345 (or optionally the bottom portion 310) comprise a transparent window or cut-out 355, 360, respectively (shown in top portion 305 and the gasket 345 for illustrative purposes). In other embodiments, in which a light emitter 365 is provided within the testing device or cartridge 300, the light emitter 365 is provided on the sensor chip 315 or imager chip carrier 320, and the transparent window or cut-out 355, 360 are not included in the top portion 305 and the gasket 345 (or optionally the bottom portion 310).

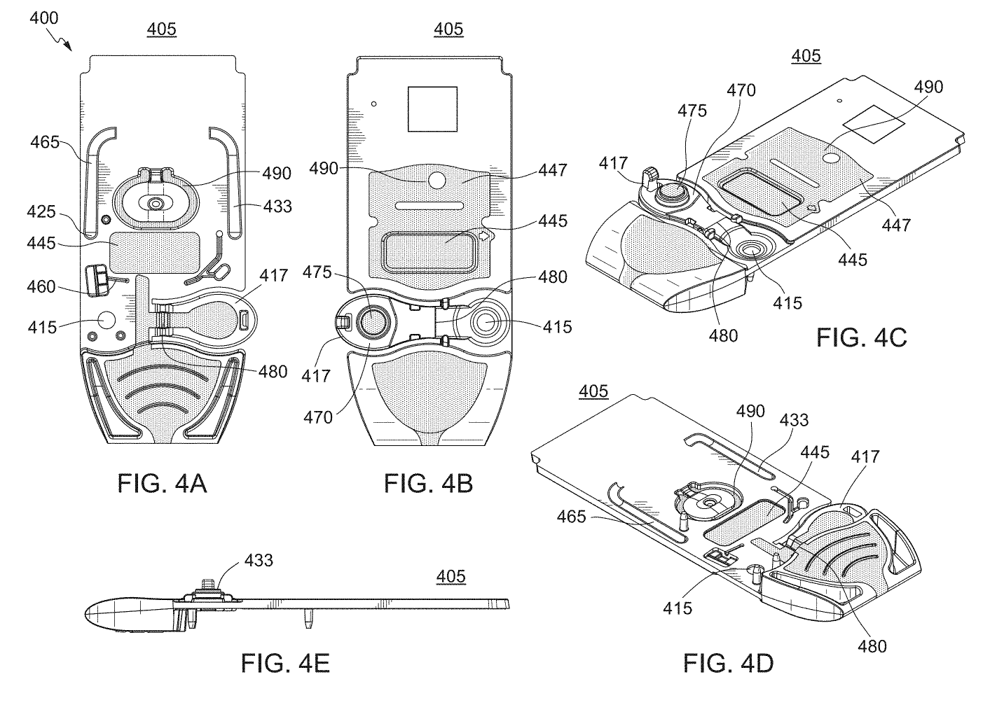

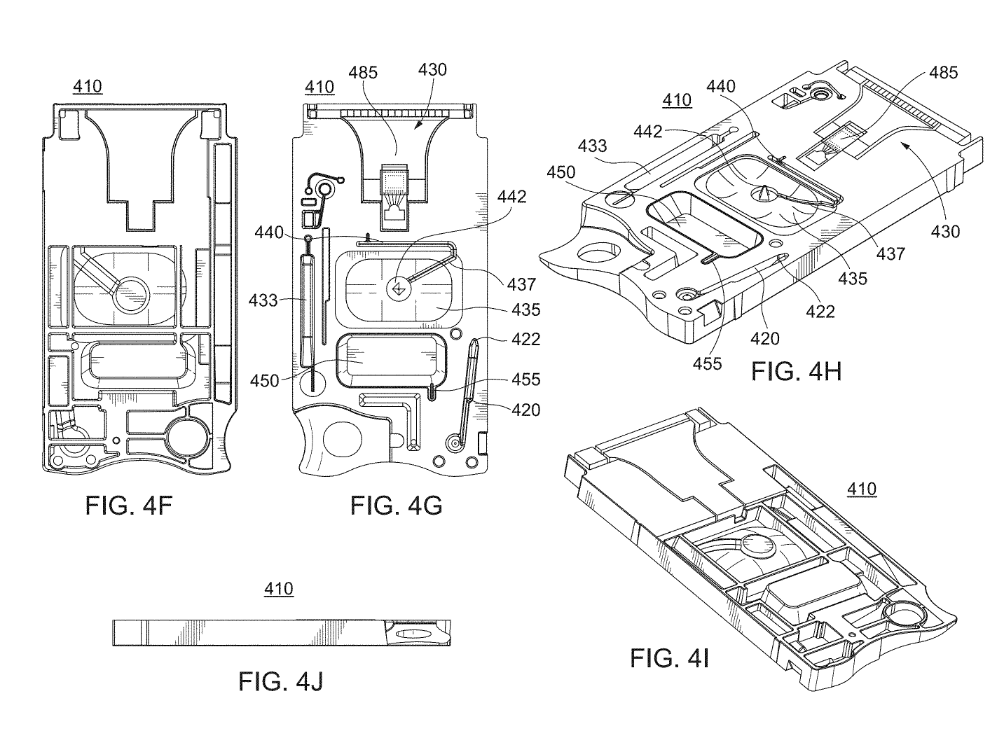

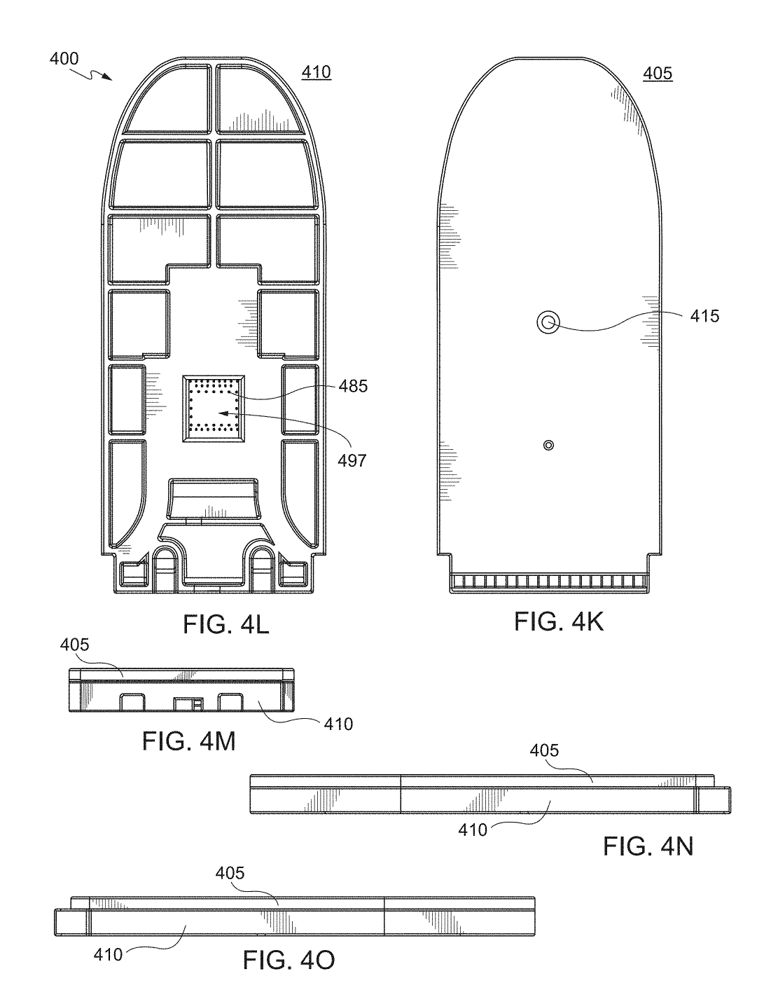

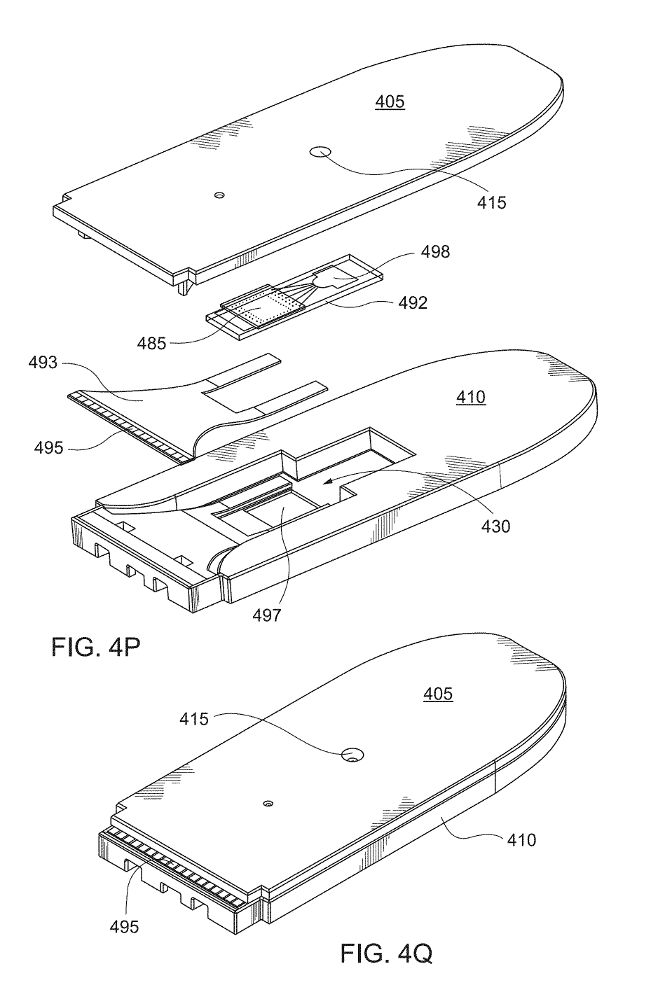

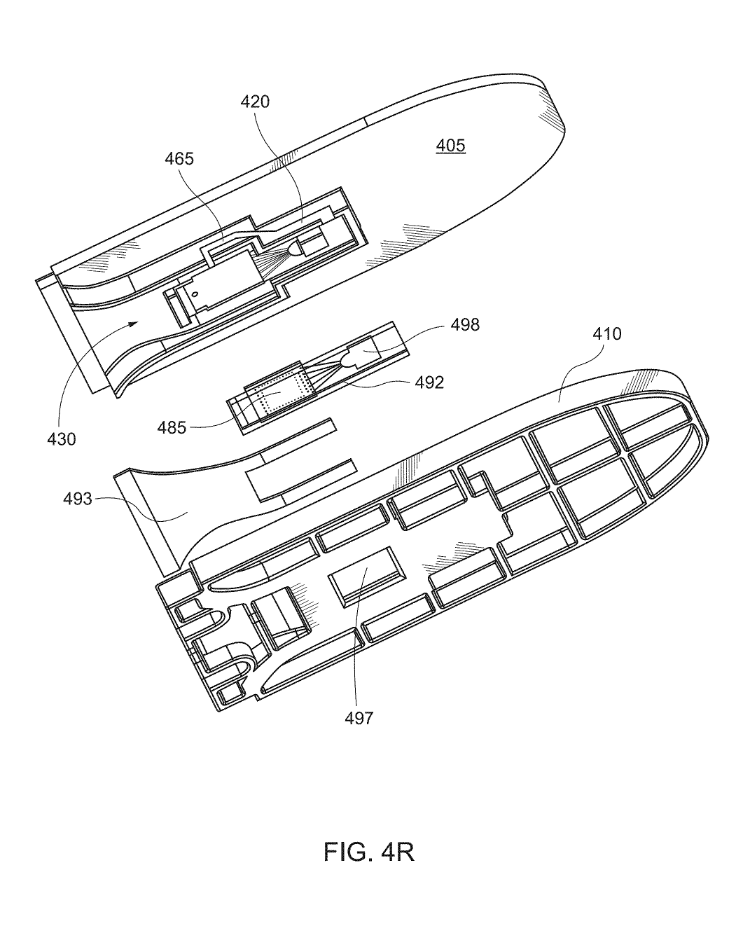

As shown in FIGS. 4A-4J, in some embodiments, the testing device or cartridge 400 (e.g., cartridge 300 as described with respect to FIG. 3) has a housing that comprises a top portion 405 (e.g., a cover) and a bottom portion 410 (e.g., a base) formed of rigid and flexible zones of material. As shown in FIGS. 4A-4J, the rigid zones (non-shaded portions) of the cover 405 and the base 410 respectively are preferably each a single contiguous zone; however, the molding process can provide a plurality of non-contiguous substantially rigid zones. The flexible zones (shaded portions) of the cover 405 and the base 410 respectively are preferably a set of several non-contiguous zones. For example, the flexible zone around a displaceable membrane may be separate and distinct from the flexible zone at a closeable sealing member. Alternatively, the flexible zones may comprise a single contiguous zone.

The testing device or cartridge 400 further comprises a sealable sample entry port 415 and a closable sealing member 417 for closing the sample entry port 415, a sample receiving chamber 420 located downstream of the sample entry port 415, an optional capillary stop 422, an optional filter 425 between the sample receiving chamber 420 and a sensor region 430 (i.e., assay region), and a waste chamber 433 located downstream of the sensor region 430. In certain embodiments, the filter 425 is configured to retain blood cells from a biological sample and permit passage of plasma into the sensor region 430. Preferably, the cross-sectional area of a portion of the sample receiving chamber 420 decreases distally with respect to the sample entry port 415. In some embodiments, a pouch (e.g., the pouch 320 described with respect to FIG. 3) is disposed in a recessed region 435 and in fluid communication with a conduit 437 leading to the sensor region 430, optionally via conduit 440. The pouch may be of the design described in U.S. Pat. No. 5,096,669 or, more preferably, in U.S. Pat. No. 8,216,529, both of which are incorporated herein by reference in their entireties. Recessed region 435 preferably includes a spike 442 configured to rupture the pouch, upon application of a force upon the pouch, for example, by reader or analyzer (e.g., analyzer 110 as described with respect to FIG. 1). Once the pouch is ruptured, the system is configured to deliver the fluid contents from the pouch into conduit 437. Movement of the fluid into the conduit 437 and to the sensor region 430 and/or within the conduit 440 may be effected by a pump, e.g., a pneumatic pump connected to the conduit(s) 437 or 440. Preferably, the pneumatic pump comprises a displaceable membrane 445 formed by a portion of a flexible zone 447 of the housing formed over a recessed region or air bladder 450. In the embodiment shown in FIGS. 4A-4J, upon repeatedly depressing the displaceable membrane 445, the device pumps via conduits 455 and 460 causing fluid from the ruptured pouch to flow through the conduit 437, optionally into the conduit 440, and over the sensor region 430 via conduit 465.

The closable sealing member 417, in some embodiments, includes a portion of the rigid zone that forms a sealing member 470, and a portion of the flexible zone that forms a seal 475. The sealing member 417 can rotate about hinge 480 and engage the seal 475 with the sample entry port 415 when in a closed position, thus providing an air-tight seal. Alternatively, an air-tight seal may be formed by contact of two flexible materials, e.g., a thermoplastic elastomer (TPE) on TPE. Optionally, the sealable sample entry port 415 also includes a vent hole (not shown). In an alternative embodiment, a portion of the rigid zone forms a sealing member, and a portion of the flexible zone forms a perimeter seal around the sample entry port, whereby the sealing member can rotate about a hinge and engage the perimeter seal when in a closed position, thus providing an air-tight seal. Alternatively, the perimeter seal may be formed by contact of two flexible materials. In yet another embodiment, the sealing member may include a slidable closure element as described in pending U.S. Pat. No. 7,682,833, the entirety of which is incorporated herein by reference.

The sensor region 430, in some embodiments, contains a sensor array comprising one or more sensors for analysis such as cell counting or determination one or more target analytes. For example, the sensor array may include an optical sensor for cell counting and optionally an electrochemical sensor for determining one or more target analytes. The optical sensor may include one or more light detectors positioned near conduit 465 for receiving light through a biological specimen, e.g., a blood sample in the conduit 465. In certain embodiments, the one or more light detectors are constructed based on similar technology (e.g., a photosensitive surface comprising an array of pixels) found in complementary metal-oxide semiconductor (CMOS) or charge-coupled device (CCD) image sensors. In some embodiments, the electrochemical sensor includes a base sensor or sensing electrode on a substantially planar chip where the sensing electrode is positioned in an auxiliary conduit (not shown) for receiving a sample mixed with a reagent.

In some embodiments, a portion of the conduit 465 forms an imaging chamber 485. For example, a portion of the conduit 465 may include (i) a planar member, (ii) a transparent planar member, and (iii) a plurality of spacer elements having an average spacer height and disposed between the planar member and the transparent planar member to form the imaging chamber 485 having an average chamber height extending between the planar member and the transparent planar member. In certain embodiments, the one or more light detectors form at least a portion of the planar member. Preferably, the portion of the conduit 465 includes a uniform width dimension in the range of about 0.5 mm to about 2 cm, a uniform length dimension in the range of about 0.5 mm to about 2 cm, and a uniform height dimension in the range of about 1.5 μm to about 35 μm, for example, about 2 μm to about 20 μm. As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent.

The analytes/properties to which the sensors respond may be selected from among particles (e.g., blood cells or microparticles), human chorionic gonadotropin, pH, partial pressure CO₂, partial pressure O₂, glucose, lactate, creatinine, urea, sodium, potassium, chloride, calcium, magnesium, phosphate, hematocrit, prothrombin time (PT), activated partial thromboblastin time (APTT), activated clotting time (ACT), D-dimer, prostate-specific antigen (PSA), creatine kinase-MB (CKMB), brain natriuretic peptide (BNP), troponin I (TnI), cardiac troponin (cTnl), human chorionic gonadotrophin, troponin T, troponin C, myoglobin, neutrophil gelatinase-associated lipocalin (NGAL), galectin-3, prostate-specific antigen (PSA), parathyroid hormone (PTH), galectin-3, aspartate aminotransferase (AST), alanine aminotransferase (ALT), albumin, total protein, bilirubin, alkaline phosphatase (ALP), and the like, and combinations thereof. In various embodiments, an optical sensor is configured to convert light received from cells within a portion of the imaging chamber to an output signal, and a processor connected to the optical sensor is configured to convert the output signal to a number count or percentage for each type of cell in the blood sample. In some embodiments, a differential blood cell count is a measurement of a number or percentage of each type of cell (e.g., white blood cells (WBCs)) that is in a whole blood sample. Cells types include erythrocytes and leukocytes and platelets. Imaging can distinguish various types of leukocytes including neutrophils, lymphocytes, granulocytes, eosinophils, basophils and monocytes. The differential blood cell count may also reveal if there are any abnormal or immature cells. Preferably, the analytes/properties are tested in a liquid sample that is whole blood, however other samples can be used including blood, serum, plasma, urine, cerebrospinal fluid, saliva and amended forms thereof. Amendments can include dilution, concentration, addition of regents such as anticoagulants and the like. Whatever the sample type, it can be accommodated by the sample entry port 415 of the cartridge 400.

In some embodiments, the cartridge 400 further comprises a portion 490 of the flexible zone 447 positioned over the recessed region 435 that is configured for being actuated upon like a pump to apply pressure within the recessed region 435. In certain embodiments, the flexible zone 447 includes a generic symbol description to indicate to the user that pressure should not be applied to the flexible zone 447 by the user. For example, the symbol may comprise an embossed circle with a crossbar. The portion 490 of the flexible zone 447 provides a surface that can accommodate an actuator feature of the analyzer (e.g., analyzer 110 as described with respect to FIG. 1) to apply a force and burst the underlying pouch in the recessed region 435. The thickness of the plastic in the portion 490 of the flexible zone 447 may be preferably from about 200 to about 800 for example about 400 μm. Essentially, the portion 490 of the flexible zone 436 should be sufficiently thin to flex easily, but sufficiently thick to maintain physical integrity and not tear.

In various embodiments, a portion of the sensor region 430 (e.g., a top surface of the substrate of a sensor), a wall of the conduit 465, and/or a wall of the sample receiving chamber 420 is coated with one or more dry reagents to amend the biological sample. The one or more dry reagents may comprise a one or more non-fluorescent or fluorescent dyes such as Eosin, Methylene Blue , Acridine Orange (also referred to as “Basic Orange 15” or “ACO”), or Astrazon Orange (also referred to as “AO” or Basic Orange 21), a component to bind to nucleic DNA in cells (e.g., blood cells such as WBCs), an anticoagulant, an antibody, an antibody fragment, an ionophore, an enzyme, a set of enzymes, a peptide with a cleavable detectable moiety, a substrate, an optical marker dye identifying a type of assay bead, and/or combinations thereof. In some embodiments, the one or more dry reagent are in the sample receiving chamber 420 and dissolve into the sample before the sample arrives at the sensor region 430. In other embodiments, a portion of the sensor region 430 (e.g., a top surface of the substrate of a sensor) includes a reagent region coated with a reactant and/or substrate for cells or an analyte of interest. The reagent region may be defined by a containment ring structure. In some embodiments, the containment ring structure is a hydrophobic ring of polyimide or another photolithographically produced layer. A microdroplet or several microdroplets (approximately 5-40 nL in size) or a series of about a 100 nanodroplets (approximately 50 to 1000 pL in size) containing the one or more dry reagents in some form may be dispensed or printed on the surface of the sensor or adjacent to the sensor. The photodefined ring structure contains this aqueous droplet allowing the reagent region to be localized to a precision of a few microns. The reagent region can be made from 0.03 to approximately 2 mm² in size. The upper end of this size is limited by the size of the conduit and sensor chip 400 in present embodiments, and is not a limitation of the invention.

The biological sample or a fluid may be passed at least once over the dry reagent, e.g., the reagent region to dissolve the reagent within the biological sample or fluid. Within a segment of the biological sample or fluid, the reagent can be preferentially dissolved and concentrated within a predetermined region of the segment. This is achieved through control of the position and movement of the segment. Thus, for example, if only a portion of a segment, such as the leading edge, is reciprocated over the reagent, then a high local concentration of the reagent can be achieved close to the leading edge. Alternatively, if a homogenous distribution of the reagent is desired, for example if a known concentration of a reagent is required for a quantitative analysis, then further reciprocation of the sample or fluid will result in mixing and an even distribution.

As shown in FIGS. 4K-4R, in alternative embodiments, the testing device or cartridge 400 (e.g., cartridge 300 as described with respect to FIG. 3) has a housing that comprises a top portion 405 (e.g., a cover) and a bottom portion 410 (e.g., a base) formed of rigid material. As shown in FIGS. 4A-4J, the rigid material of the cover 405 and the base 410 respectively are preferably each a single contiguous zone; however, the molding process can provide a plurality of non-contiguous substantially rigid zones. The testing device or cartridge 400 further comprises a sample entry port 415, a sample receiving chamber 420 located downstream of the sample entry port 415, and a conduit 465 fluidically connecting the sample receiving chamber 420 to a sensor region 430 (i.e., assay region). Sample motion in the sample receiving chamber 420, conduit 465, and sensor region 430 may be controlled by capillary action where fluidic paths and conduits are dimensioned to promote capillary action. The surfaces of the fluidic paths and/or conduit may also be treated to make them more or less hydrophilic and hydrophobic to further promote capillary action using established techniques known in the art.

In some embodiments, the sensor region 430 comprises at least one microfabricated sensor chip 492 disposed on a imager chip carrier 493 (e.g., a substrate) with electrical contacts 495. The sensor chip 492 and imager chip carrier 493 may be positioned in the sensor region 430 and configured to generate electric signals based on, for example, light transmitted through an imaging chamber 485 of the sensor chip 492 and a biological specimen, e.g., a blood sample from a patient. In some embodiments, in which a light emitter is provided by an external element such as the analyzer (e.g., the reading apparatus 110 as discussed with respect to FIG. 1), the top portion 405 or bottom portion 410 comprise a transparent window or cut-out 497 (shown in bottom portion 410 for illustrative purposes). In other embodiments, in which a light emitter 498 is provided within the testing device or cartridge 400, the light emitter 498 is provided on the sensor chip 492 or imager chip carrier 493, and the transparent window or cut-out 497 is not included in the top portion 405 or bottom portion 410. In some embodiments, a portion of the conduit 465 forms the imaging chamber 485. For example, a portion of the conduit 465 may include (i) a planar member, (ii) a transparent planar member, and (iii) a plurality of spacer elements having an average spacer height and disposed between the planar member and the transparent planar member to form the imaging chamber 485 having an average chamber height extending between the planar member and the transparent planar member. In certain embodiments, the one or more light detectors form at least a portion of the planar member. Preferably, the portion of the conduit 465 includes a uniform width dimension in the range of about 0.5 mm to about 2 cm, a uniform length dimension in the range of about 0.5 mm to about 2 cm, and a uniform height dimension in the range of about 1.5 μm to about 35 μm, for example, about 2 μm to about 20 μm.

The analytes/properties to which the sensors respond may be selected from among particles (e.g., blood cells or microparticles), human chorionic gonadotropin, pH, partial pressure CO₂, partial pressure O₂, glucose, lactate, creatinine, urea, sodium, potassium, chloride, calcium, magnesium, phosphate, hematocrit, prothrombin time (PT), activated partial thromboblastin time (APTT), activated clotting time (ACT), D-dimer, prostate-specific antigen (PSA), creatine kinase-MB (CKMB), brain natriuretic peptide (BNP), troponin I (TnI), cardiac troponin (cTnI), human chorionic gonadotrophin, troponin T, troponin C, myoglobin, neutrophil gelatinase-associated lipocalin (NGAL), galectin-3, prostate-specific antigen (PSA), parathyroid hormone (PTH), galectin-3, aspartate aminotransferase (AST), alanine aminotransferase (ALT), albumin, total protein, bilirubin, alkaline phosphatase (ALP), and the like, and combinations thereof. In various embodiments, an optical sensor is configured to convert light received from cells within a portion of the imaging chamber to an output signal, and a processor connected to the optical sensor is configured to convert the output signal to a number count or percentage for each type of cell in the blood sample. In some embodiments, a differential blood cell count is a measurement of a number or percentage of each type of cell (e.g., white blood cells (WBCs)) that is in a whole blood sample. Cells types include erythrocytes and leukocytes and platelets. Imaging can distinguish various types of leukocytes including neutrophils, lymphocytes, granulocytes, eosinophils, basophils and monocytes. The differential blood cell count may also reveal if there are any abnormal or immature cells. Preferably, the analytes/properties are tested in a liquid sample that is whole blood, however other samples can be used including blood, serum, plasma, urine, cerebrospinal fluid, saliva and amended forms thereof. Amendments can include dilution, concentration, addition of regents such as anticoagulants and the like. Whatever the sample type, it can be accommodated by the sample entry port 415 of the cartridge 400.

In various embodiments, a portion of the sensor region 430 (e.g., a top surface of the substrate of a sensor), a wall of the conduit 465, and/or a wall of the sample receiving chamber 420 is coated with one or more dry reagents to amend the biological sample. The one or more dry reagents may comprise Acridine Orange (also referred to as “Basic Orange 15” or “ACO”), Astrazon Orange (also referred to as “AO” or Basic Orange 21), a component to bind to nucleic DNA in cells (e.g., blood cells such as WBCs), an anticoagulant, an antibody, an antibody fragment, an ionophore, an enzyme, a set of enzymes, a peptide with a cleavable detectable moiety, a substrate, an optical marker dye identifying a type of assay bead, and/or combinations thereof. In some embodiments, the one or more dry reagent are in the sample receiving chamber 420 and dissolve into the sample before the sample arrives at the sensor region 430. In other embodiments, a portion of the sensor region 430 (e.g., a top surface of the substrate of a sensor) includes a reagent region coated with a reactant and/or substrate for cells or an analyte of interest. The reagent region may be defined by a containment ring structure. In some embodiments, the containment ring structure is a hydrophobic ring of polyimide or another photolithographically produced layer. A microdroplet or several microdroplets (approximately 5-40 nL in size) or a series of about a 100 nanodroplets (approximately 50 to 1000 pL in size) containing the one or more dry reagents in some form may be dispensed or printed on the surface of the sensor or adjacent to the sensor. The photodefined ring structure contains this aqueous droplet allowing the reagent region to be localized to a precision of a few microns. The reagent region can be made from 0.03 to approximately 2 mm² in size. The upper end of this size is limited by the size of the conduit and sensor chip 400 in present embodiments, and is not a limitation of the invention. The biological sample or a fluid may be passed over the dry reagent by capillary action, e.g., the reagent region, to dissolve the reagent within the biological sample or fluid.

### Imaging Device

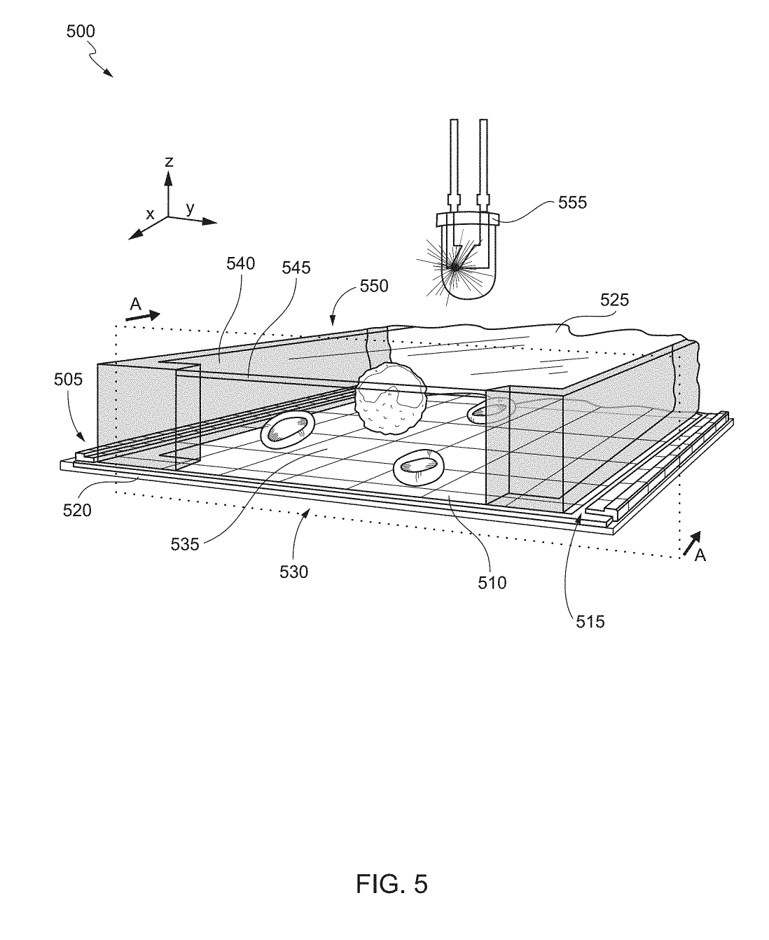

In order to implement lensless microscopy in point-of-care applications (e.g., the analyzer and cartridge systems described with respect to FIGS. 1, 2, 3, and 4A-4R), several aspects of the present invention are directed to imaging devices comprising: (i) one or more light detectors, (ii) an imaging chamber, and (iii) one or more light emitters. Various embodiments of imaging devices are illustrated by the examples shown in FIGS. 5-10 and 11A-11I. In some embodiments, an imaging device 500 comprises a light detector such as an imager chip 505 having an inner surface or photosensitive surface 510 presented by an imaging integrated circuit 515 formed on a substrate. The imaging integrated circuit 515 (e.g., a very-large-scale integrated (VLSI) circuit) may have a high-resolution photosensitive array including a multi-dimensional array of pixels presented at its surface 510 and non-photosensitive supporting circuitry for processing and readout. The imaging integrated circuit 515 may be electrically and mechanically attached to a sensor chip 520, which is a printed circuit board whose components connect to one or more electrical connections (e.g., an electrical connection comprising a plurality of discrete contacts) that are capable of connecting the photosensitive surface 510 to one or more conductive pins such as a temporary electrical connector of an analyzer (e.g., analyzer 110 as described with respect to FIG. 1).

The multi-dimensional array of pixels may be light sensors or photodetectors formed of semiconductor materials used in very-large-scale or larger integrated circuits. The defining property of a semiconductor material is that it can be doped with impurities that alter its electronic properties in a controllable way; in some embodiments, the array is formed substantially of a crystalline inorganic solid such as silicon; and in other embodiments the array is formed substantially of a compound semiconductor comprised of elements of at least two different species. The compound semiconductor may be comprised of elements in groups 13-15 (old groups III-V), for example of elements from group 13 (old group III, boron, aluminum, gallium, indium) and from group 15 (old group V, nitrogen, phosphorus, arsenic, antimony, bismuth). The range of possible formulae for the compound semiconductor may include binary (two elements, e.g., gallium (III) arsenide (GaAs)), ternary (three elements, e.g., indium gallium arsenide (InGaAs)), and quaternary (four elements, e.g., aluminum gallium indium phosphide (AlInGaP)) alloys. In some embodiments, the array of pixels are light sensors or photodetectors such as PD(s), e.g., a silicon photo PIN diode(s) having an undoped intrinsic semiconductor region sandwiched between a p-type semiconductor region and an n-type semiconductor region. Alternatively, other light sensors or detectors with or without filters to control wave lengths may be used without departing from the spirit and scope of the present invention. The spectral response of the multi-dimensional array of pixels may be in the range of 300 nm to 1000 nm. This provides the capability to cover a wide spectrum of LED wavelengths. The size of the photosensitive surface 510 may be selected to fit with other components (e.g., the conduits or sensor region) of the testing device, e.g., the photosensitive surface 510 available as surface mount diode (SMD) and chip scale packaging (CSP) may be used to fit a variety of testing devices. In some embodiments, the sensor chip 520 may have a width from about 1 mm to about 20 mm and a length from about 1 mm to about 20 mm (e.g., a width of about 5 mm and a length of about 6 mm), in order to accommodate a low profile photosensitive surface 510 that has the industry standard 2.0 mm×1.25 mm footprint, which provides high efficiency light detection and low power consumption. In accordance with various aspects, the sensitivity of the photosensitive surface 510 is within the range of 0.5 uA/cm²-4 uA/cm², for example substantially 1 uA/cm².

As described herein, the multi-dimensional array of pixels form part of a high-resolution photosensitive array. The term “high-resolution” as used herein refers to a resolution that equals or exceeds the resolution of standard lens-based optical microscopes. The resolution of a standard lens-based optical microscopes is defined as the shortest distance between two points on a specimen that can still be distinguished by the observer or camera system as separate entities. For example, depending on the context of the application, high-resolution means less than 5 μm, less than 2 μm, less than 1 μm, less than about 0.5 μm, or even less. Resolution in an optical sensor is primarily determined by the pixel size of the photosensitive array. Some photosensitive arrays have many million square pixels each slightly more than 1 μm on a side, resulting in a resolution of about 1 μm; the resolution achievable will improve with decreasing pixel sizes, theoretically exceeding, for example, 1 billion pixels, each as small as 200 nm or less on a side, as the design and fabrication techniques of integrated circuits or other devices improve. Pixels per inch (PPI) or pixels per centimeter (PPCM) are measurements of the pixel density of the optical sensor. The resolution of the optical sensor is the count of pixels that contribute to the final image and is typically measured in megapixels (meaning millions of pixels). For example, a photosensitive array comprising 1280×720 pixels has 921,600 pixels or less than 1 mega pixel resolution, and a photosensitive array comprising 1920×1080 pixels has 2,073,600 pixels or about 2.1 mega pixel resolution. In some embodiments, the photosensitive surface 510 is comprised of an array of pixels having at least 5 mega pixel resolution with at least a 150 ppi pixel density. In some embodiments, each pixel of the array has a length and width of equal to or less than 10 μm, equal to or less than 5 μm. equal to or less than 1 μm, equal to or less than 500 nm, or equal to or less than 250 nm. In certain embodiments, each pixel of the array has an area of about 0.9 μm², 1.1 μm², 1.4 μm², or 1.8 μm². In terms of ranges each pixel of the array may have an area of less than about 10.0 μm², less than about 5.0 μm², less than about 2.0 μm², for example, from about 0.5 μm² to about 1.5 μm².

Micro-fabrication techniques (e.g., photolithography and plasma deposition) may be utilized for construction of multilayered sensor structures in confined spaces. In some embodiments, the imaging integrated circuit 515 is fabricated to include a CCD. In other embodiments, the imaging integrated circuit 515 is fabricated using CMOS technology. CCDs have advantages for contact optical microscopy applications, including the ability to detect light over the full exposed surface of the chip (100% fill factor), though they have slower readout speeds relative to CMOS due to requirement for sequential transfer of charge from light-sensing (parallel register) to readout (serial register) elements. Various configurations of CCD can be used: full-frame architecture may be used to maximize the proportion of the chip available for imaging, but requires an external shutter to prevent image smearing during readout; whereas frame-transfer architecture avoids image smearing, but in the process requires a masked, non-photosensitive area of the parallel register of about the same size as the photosensitive area of the parallel register, with the result that the imaging integrated circuit has about half the photosensitive area of a full-frame architecture. Because of the small area of the individual pixels in the arrays used in accordance with various aspects discussed herein, the charge collected in each pixel will be small under many imaging conditions; however, as the specimen is in contact, or nearly in contact, with the pixel, the pixel's effective acceptance angle for photons emanating from the specimen is larger than that achieved by lenses in conventional microscopy. In some CCD embodiments, to increase the sensitivity further, CCDs of any architecture additionally employ electron multiplying gain, in which high clock voltages applied to an extended region of the serial register(s) amplify the charge of each pixel as it is shifted to the output node(s).

CMOS devices have alternative advantages for these applications, including less expensive fabrication, signal processing by electronic elements embedded in individual pixels, and the ability to read out independently-addressed pixel values individually without sequential transfer. In some CMOS embodiments, thinned back-side illuminated arrays are used; though previously requiring expensive and complex fabrication methods, these may be fabricated cheaply using bonded wafer processes such as those that use silicon-on-insulator substrates with a buried oxide layer as an etch-stop to yield a uniformly optimally thinned light-absorbing back layer (see as an example, U.S. Pat. No. 7,425,460, which is incorporated herein by reference). Light entering ordinary (front-side illuminated) imaging integrated circuits typically passes through overlying layers that scatter light and whose metal circuit elements block the underlying photosensitive layer; in back-side illuminated imaging integrated circuits the photosensitive layer is close to the surface, above the metal circuit-bearing layers, typically resulting in less light blocking (larger “fill factors”) and consequently higher effective quantum efficiency.

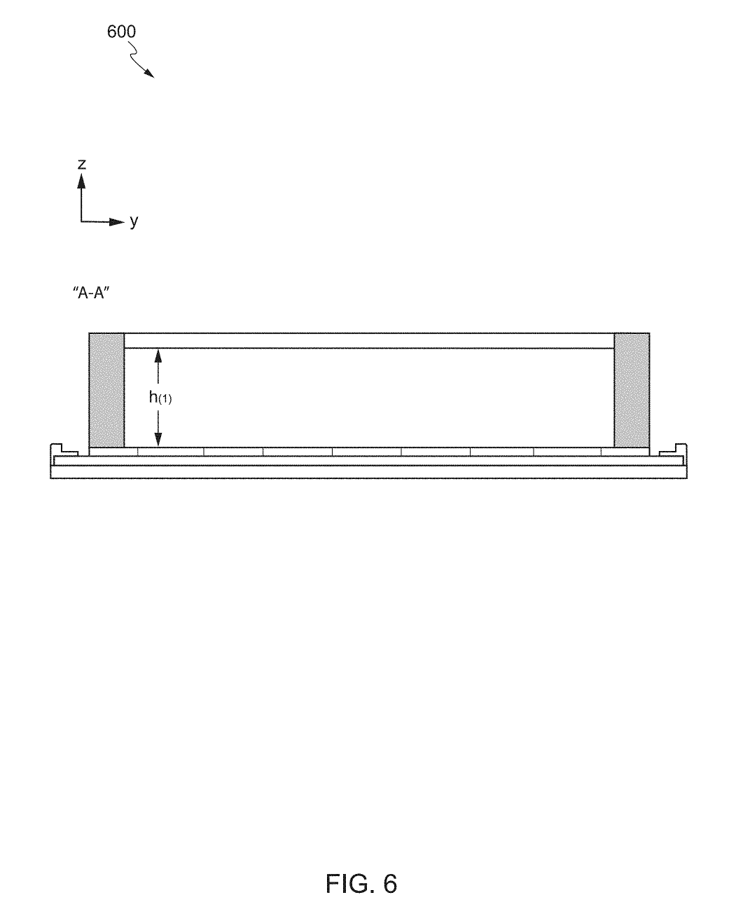

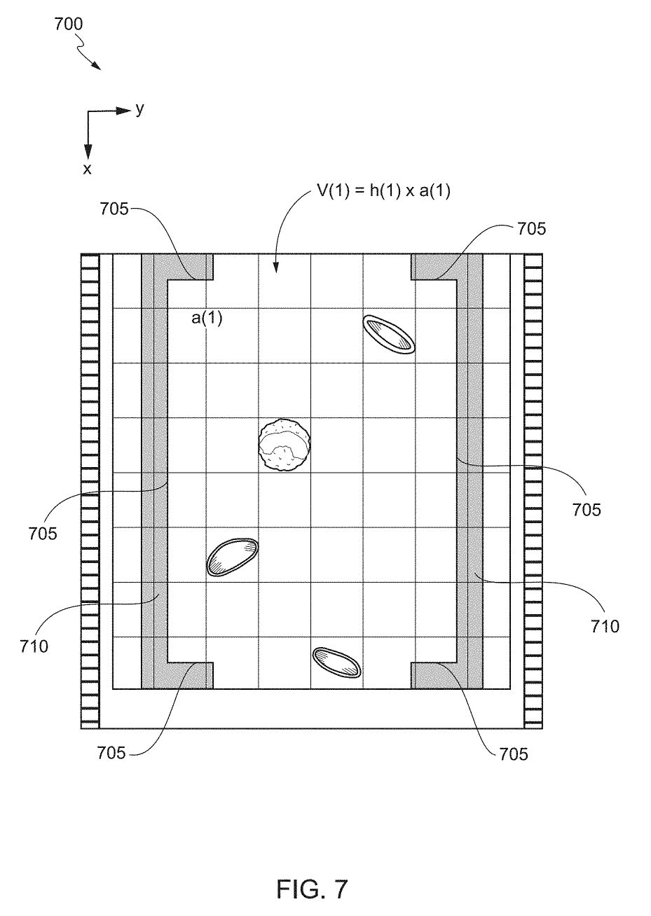

In various embodiments, the imaging device 500 further comprises a sample testing conduit 525 fluidically connected to a sample receiving chamber (e.g., the sample receiving chamber 420 of FIGS. 4A-4R). A portion of the sample testing conduit 525 may include: (i) a planar member 530 having an inner surface 535 (e.g., the imager chip 505 having the photosensitive surface 510) and (ii) a transparent planar member 540 having an inner surface 545 to form an imager chamber 550 having an average chamber height extending between the planar member and the transparent planar member. The imaging chamber 550 structured to obtain images of at least a portion of a sample residing in the imaging chamber 550. In some embodiments, the imaging chamber is structured to obtain images of cells within the sample (e.g., a monolayer of red blood cells and/or white blood cells). In other embodiments, the imaging chamber is structured to obtain images of one or more assay beads used in the performance of an analytical test (e.g., a qualitative or semi-quantitative analytical test for a target analyte within the sample). FIG. 6 shows a cross-section of an imaging chamber 600 having a predetermined height (h1) measured on the Z-axis. In some embodiments, the chamber height (h1) is selected to accommodate the analysis of cells (e.g., the formation of a monolayer of cells) and will be from about 2 μm to about 20 μm, from about 2 μm to about 6 μm, or from about 3 μm to about 5 μm, for example about 4 μm, about 2 μm, or about 6 μm. In other embodiments, the chamber height (h1) is selected to accommodate the analysis of assay beads and will be from about 0.5 μm to about 40 μm, from about 0.5 μm to about 20 μm, or from about 2 μm to about 10 μm, for example about 4 μm, about 8 μm, or about 10 μm. FIG. 7 shows a top planar view of an imaging chamber 700 having an area (a1) measured on the X-Y plane and a volume ((v1)=(h1)×(a1)) measured on the x, y, and z axis. The lateral boundaries 705 of the imaging chamber 700 may be defined, for example, by structural features 710 (e.g., sides of the conduit, glue lines, or hydroscopic material disposed on a planar member surface that inhibit lateral travel) extending between the inner surfaces of the planar member and the transparent planar member, respectively.

The imaging chamber 500, 600, 700 is typically sized to hold about 0.2 to about 2.0 μL of sample, but the imaging chamber 500, 600, 700 is not limited to any particular volume capacity, and the capacity can vary to suit the analysis application. For example, sized to create a monolayer of red blood cells or white blood cells for cell identification and counting. In some embodiments, the imaging chamber 500, 600, 700 is operable to quiescently hold a liquid sample. The term “quiescent” is used herein to describe that the sample is deposited within imaging chamber 500, 600, 700 for analysis, and is not purposefully moved during the analysis. To the extent that motion is present within the blood sample, it will predominantly be due to Brownian motion of formed constituents within the blood sample, which motion is not disabling of the use of this invention. However, in other embodiments, the imaging chamber 500, 600, 700 is operable to actively hold a liquid sample. The term “actively” is used herein to describe that the sample is deposited within imaging chamber 500, 600, 700 for analysis, and is purposefully moved during the analysis (e.g., via a pump such as a displaceable membrane 426 formed by a portion of a flexible zone 427 as described with respect to FIGS. 4A-4R).

As shown in FIG. 5, in various embodiments, the imaging device 500 further comprises a light emitter 555 positioned near the sample testing conduit 525 (e.g., on a side of the conduit or above the conduit). In some embodiments, the light emitter 555 is provided by an external element such as the analyzer (e.g., the reading apparatus 110 as discussed with respect to FIG. 1). In other embodiments, the light emitter 555 is provided on the imager chip or imager chip carrier within the testing device or cartridge (e.g., the testing device or cartridge 110 as discussed with respect to FIG. 1). In some embodiments, the light emitter 555 is positioned so that a path of light from the light emitter 555 to the photosensitive surface 510 of the sensor chip 520 is at an angle of 45 degrees or more to the photosensitive surface 510. In some embodiments, the light emitter 555 is positioned so that a path of light from the light emitter 555 to the photosensitive surface 510 is at an angle of at most 45 degrees to the photosensitive surface 510. In some embodiments, the light emitter 555 is positioned so that a path of light from the light emitter 555 to the photosensitive surface 510 is approximately perpendicular or parallel to the photosensitive surface 510.

Images of the specimen can be obtained in the presence of the light emitter 555. The light emitter 555 may produce light of at least one wavelength for which the imaging integrated circuit 515 is responsive. In some embodiments, the light emitter 555 includes a laser and the predetermined wavelength is the substantially monochromatic wavelength of the laser. In some embodiments, the light emitter 555 includes a blackbody and the predetermined wavelength band is a segment of the electromagnetic spectrum which the blackbody is suitably efficient at producing, with or without use of a bandpass spectral filter interposed between the light emitter 555 and the specimen. In some embodiments, the light emitter 555 comprises one or more light-emitting diodes, for example, an organic light-emitting diode array, oriented so as to produce light in the predetermined wavelength band or bands. In some embodiments, the light emitter 555 is continuous. In some embodiments, the light emitter 555 is pulsed. In some embodiments, the light emitter 555 is polarized. In some embodiments, the light emitter 555 includes any ambient, incandescent, or fluorescent light source. In certain embodiments in which a fluorescent dye is used as a reagent, the light emitter 555 includes a fluorescence illuminator. In some embodiments, the light emitter 555 is structured, such as a periodic grating of bright bars. In conjunction with appropriate oblique, pulsed, polarized, structured, or other forms of illumination, some embodiments can generate additional useful information corresponding to methods known in the art of microscopy, including but by no means limited to dark field, fluorescence, fluorescence lifetime, optical tomography, and polarization microscopy. In some embodiments, the specimen is itself the light emitter 555; for example through chemi-luminescence, or in an instance where the photosensitive array is treated to render the pixels sensitive to radiation emitted by a radioactive specimen. In some embodiments, there may be additional light emitters capable of operating as any of the aforementioned light emitters.

The spectra of the light source(s) may lie in any predetermined region of the electromagnetic spectrum detectable using photosensitive arrays, with or without specialized treatments to extend the effective ranges of wavelengths detectable by such arrays. In some embodiments, the predetermined wavelength or wavelength band is in the infrared spectrum. In some embodiments, the predetermined wavelength or wavelength band is in the ultraviolet spectrum. In some embodiments, the predetermined wavelength or wavelength band is in the visible spectrum. In certain embodiments, the light emitter 555 is configured to transmit light through the imaging chamber 550 to the sensor chip 520 at a wavelength from about 300 nm to about 1000 μm. In other embodiments, the light emitter 555 is configured to transmit light through the imaging chamber 550 to the sensor chip 520 at a plurality of wavelengths from about 300 nm to about 1000 μm.

In various embodiments, the light emitter 555 is located adjacent to the imaging chamber 530 (i.e., near the transparent planar member, for example within about 1 mm, about 2 mm, or about 3 mm). In certain embodiments, the testing cartridge or test device comprises a housing, and the sensor chip 520, the sample testing conduit 525, and the light emitter 555 are housed within the housing. For example, the test cartridge may comprise the light emitter 555, and the light emitter 555 may be electrically connected to at least one of a plurality of connector contacts, and the light emitter 555 may be configured to transmit light through the portion of the imaging chamber 550 to the sensor chip 520 at one or more wavelengths from about 300 nm to about 1000 μm. In other embodiments, the analyzer comprises the light emitter 555, and the test cartridge further comprises a housing comprising a window adjacent to the sample testing conduit 525 for illuminating the portion of the imaging chamber 550, and the test cartridge is insertable into the port of the analyzer such that the light emitter 555 is aligned over the window and the portion of the imaging chamber 550 to transmit light through the portion of the imaging chamber 550 to the sensor chip 520 at one or more wavelengths from about 300 nm to about 1000 μm.

In some embodiments, the light emitter 555 includes individually controlled light-emitting diodes (LEDs) selected for their spectral emission characteristics and their uniformity of emitted light, and positioned so as to facilitate the analyses contemplated. In some embodiments, the light emitter 555 is positioned so as to uniformly illuminate the imaging chamber 550. The LEDs may be controlled, for example, by an embedded controller incorporated within the cartridge or the analyzer. The LEDs may be controlled, for example, either singly or in groups so as to facilitate the analyses to be contemplated, including but not limited to conventional microscopy wherein the illuminator, the specimen and the imaging system are substantially aligned, and dark-field microscopy wherein the specimen is illuminated from an angle outside the acceptance angle of the pixel. In addition, through appropriate selection of the LEDs in the light emitter 555, the contemplated imaging devices can be used for, but not be limited to, e.g., color imaging, fluorescence microscopy, polarization microscopy, infra-red and ultra-violet microscopy. Some embodiments will incorporate multiple light emitters 555, each of which may have different characteristics so as to facilitate the conduct of a wider range of analyses. In some embodiments the light emitter 555 will be easily interchangeable. In some embodiments the light emitter 555 includes organic LED (OLED) or active matrix organic LED (AMOLED) panel with selective addressing. Some embodiments facilitate both uniform specimen illumination and rapid illumination variation so as to facilitate analyses yet to be contemplated with both stationary and moving specimens. In some embodiments, an AMOLED panel is used to illuminate the specimen through appropriate control of the panel photoemitters. In some examples, the light emitter 555 can include LEDs, organic LED panels, fluorescent panels, ultraviolet sources, ambient illumination such as sunlight or room light, incandescent sources, or any other light source, including none, e.g., for chemiluminescent specimens, and combinations of these examples. Configurations of the light emitter 555 include, but are not limited to, flat panels, rectangular or other grid layouts of sources, movable sources, multi-color sources, and sources affixed to the inside or a hemispherical shell mounted over the imaging chamber 550 with the center of the chamber as the center of the shell, or combinations of them. Control of the light emitter 555 may include, but not be limited to, steady illumination, selectively exciting one or a plurality of light emitters 555 simultaneously or in sequence, controlling the intensity of any one or a plurality of light emitters 555, controlling each or a plurality of light emitters 555 so as to have a specific temporal illumination pattern, or using any one or any combination of them and others (including future technologies). The controller for the light emitter 555 may include, but not be limited to, a manual controller such as a switch or knob, an automated embedded computing system, an external computing system such as onboard the analyzer, an external computing system such as a desktop or laptop computer, or a combination of the foregoing.

### Imaging Cells

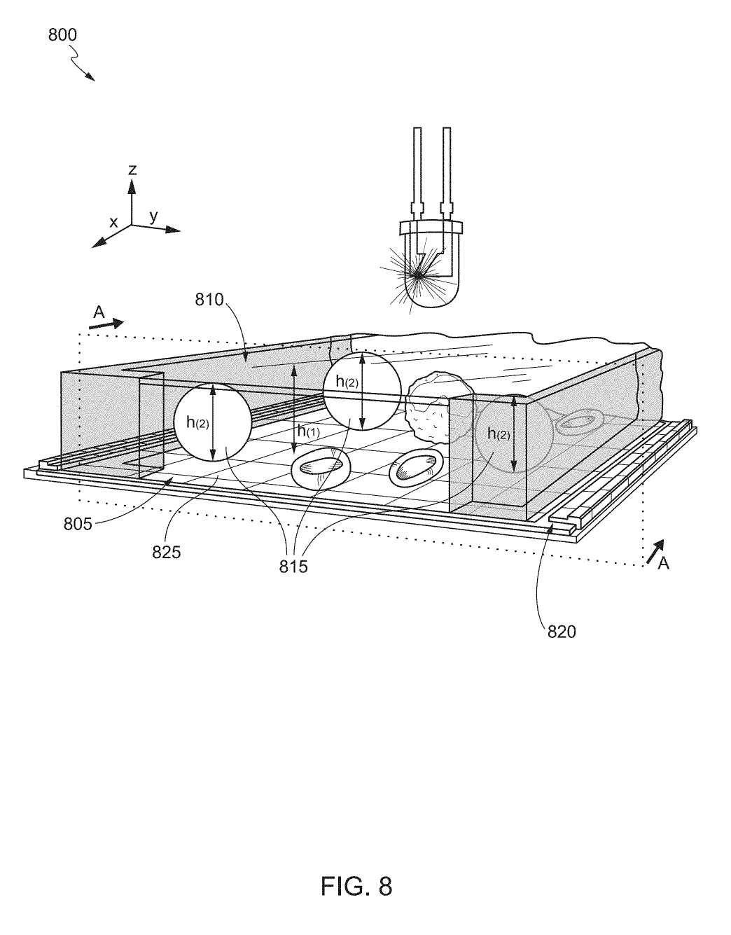

As shown in FIG. 8, an imaging chamber 800 comprises a first planar member 805, a second planar member 810, and a plurality of spacer elements 815 disposed between the first planar member 805 and the second planar member 810. A height (h1) of the imaging chamber 800 is predetermined such that the sample residing within the imaging chamber 800 will travel laterally within the imaging chamber 800 via capillary forces. In various embodiments, at least one of the first planar member 805 and the second planar member 810 is transparent (e.g., a transparent planar member). Transparent plastic films comprising acrylic or polystyrene are examples of acceptable materials for the first planar member 805 and the second planar member 810. In some embodiments, at least a portion of the first planar member 805 or the second planar member 810 (e.g., a planar member) is formed from the imager chip 820. For example, typically optical sensors have a protective window over the photosensitive surface 825. However, in certain embodiments, the optical sensors do not comprise a protective window and instead the imager chip 820 is windowless in order for the sample to come close enough to the photosensitive surface 825 to achieve high resolution, as defined herein, without computational image processing. For portions of the sample within half a pixel width of the photosensitive surface 825, the resolution of the image is limited by the size of the pixels making up the photosensitive surface 825. For example, when a point on the sample (e.g., a cell) is less than half a pixel width from the center of the closest pixel, nearly all the light emitted or scattered from that point toward the array will predominantly be incident on, and therefore excite, only the closest pixel; under these conditions, the resolution may be determined by the pixel size or, more precisely, by the size of a circle of equivalent area (e.g., about a 450 nm resolution for a 400 nm×400 nm pixel), although resolution may be further enhanced by computation, sample flow, or other means. A near-field criterion may be considered to be reached, for example, when the distance between the photosensitive surface and the sample is less than the wavelength of interest. No lenses or any other optical components are required to achieve these conditions, and thus to achieve such pixel-limited resolution.

In other embodiments, the photosensitive surface 825 is treated with one or more thin layers. The layers may be considered thin when the aggregate thickness of such layers as applied to the photosensitive surface 825 still allows for the near-field criterion described herein to be satisfied or substantially satisfied. In several embodiments, the layers are thin enough for the sample to come within half a pixel width of the photosensitive surface 825. In some embodiments, the layers are thin enough in the direction of the optical path so that the total distance that the optical path takes through the layers is no more than about the wavelength of interest. In some embodiments, a thin layer of transparent chemically resistant material coats the photosensitive surface 825. Such a thin-film substrate may be any sufficiently transparent and insulating material, including but not limited to silicon oxide, titanium oxide, aluminum oxide, tantalum oxide, magnesium fluoride, lanthanum fluoride, aluminum fluoride, silicon nitride, and silicon oxynitride; and it may be deposited by a variety of means including but not limited to magnetron sputtering, chemical vapor deposition, thermal or vacuum arc plasma evaporation. In some embodiments, the substrate is a dielectric thin film acting as an interference filter, thereby restricting the spectral sensitivity of the underlying pixels as appropriate to a given application. In some embodiments, the substrate is used to effect certain forms of color imaging. In certain embodiments, the substrate is substantially transmissive to a portion of a predetermined wavelength band, such as a band-pass filter. In other embodiments such as for fluorescence or emission microscopy, the substrate is substantially transmissive to an alternative predetermined wavelength band which corresponds to the wavelength band produced by fluorescence, emission, or in other ways, of the sample. In some embodiments, the substrate includes a dielectric thin film acting as an anti-reflection coating. In some embodiments, there are multiple substrates situated in close contact to each other. In some embodiments, the photosensitive surface 825 is silanized so as to decrease adhesion between the surface and the sample. In some embodiments, the chemically resistant material includes diamond, deposited in a suitably thin layer as, for example, by chemical vapor deposition. In some embodiments, the chemically resistant material includes Al₂O₃ or Si₃N₄, deposited in a suitably thin layer as, for example, by chemical vapor deposition. Such materials can impart more robust characteristics to the photosensitive surface 825, allowing for ease of cleaning as well as protection of the surface from abrasive samples. In some embodiments, a passivation layer, typically of Si3N4, coats the imaging integrated circuit, resulting in reduced conductivity when used with metallic or other conductive samples such as salt solutions. In some embodiments, a thin layer of polarizing material coats the photosensitive surface 825. In some embodiments, a thin layer of absorptive material coats the photosensitive surface 825. In some embodiments, a thin layer of interference material coats the photosensitive surface 825. In some embodiments, a thin layer of surface plasmon generating material coats the photosensitive surface 825. Technology is available to deposit such layers as a thin film and in arbitrary pixel-by-pixel patterns.

In various embodiments, the plurality of spacer elements 815 are any structure that is disposable between the first planar member 805 and the second planar member 810, and operable to space the first planar member 805 and the second planar member 810 apart from one another and maintain the chamber height (h1). The height of each of the plurality of spacer elements 815 typically do not equal one another exactly, but are substantially equal and within commercially acceptable tolerance for spacing means used in similar analysis apparatus. As such the height of the plurality of spacer elements 815 is characterized as the average spacer height (h2). In some embodiments, the average spacer height (h2) is selected to accommodate the analysis of cells (e.g., the formation of a monolayer of cells) and will be from about 2 μm to about 20 μm, from about 2 μm to about 6 μm, or from about 3 μm to about 5 μm, for example about 4 μm, about 2 μm, or about 6 μm. In other embodiments, the average spacer height (h2) is selected to accommodate the analysis of assay beads and will be from about 0.5 μm to about 40 μm, from about 0.5 μm to about 20 μm, or from about 2 μm to about 10 μm, for example about 4 μm, about 8 μm, or about 10 μm. The height of the spacer elements can be determined by any known analytical technique typically used to measure the height or size of an object, such as flow cytometry, laser device, SEM imaging, particle size analyzer, etc. As used herein, the “average spacer height” means the average height of at least 90% of the spacer elements used to construct the chamber. Average is understood as a calculated “central” value of a set of numbers (the sum of the set of numbers divided by the count), where the set of numbers is the set of height values for at least 90% of the spacer elements used to construct the chamber.

In some embodiments, the plurality of spacer elements 815 are spherical beads (e.g., uniform polymer, silica or magnetic microsphere products for diagnostic, research and flow cytometry applications commercially available from, for example, Bangs Laboratories, Inc. In some embodiments, the plurality of spacer elements 815 are pillar structures fabricated on: (i) at least a portion of a surface of the first planar member 805 (e.g., the photosensitive surface 825 of the sensor chip 820), and/or (ii) at least a portion of a surface of the second planar member 810. In some embodiments, the plurality of spacer elements 815 are structures embossed on: (i) at least a portion of a surface of the first planar member 805 (e.g., the photosensitive surface 825 of the sensor chip 820), and/or (ii) at least a portion of a surface of the second planar member 810. In some embodiments, the plurality of spacer elements 815 are formed directly (physical contact between the objects) on: (i) at least a portion of a surface of the first planar member 805 (e.g., the photosensitive surface 825 of the sensor chip 820), and/or (ii) at least a portion of a surface of the second planar member 810. In some embodiments, the plurality of spacer elements 815 are formed indirectly (no physical contact between the objects) on: (i) at least a portion of a surface of the first planar member 805 (e.g., the photosensitive surface 825 of the sensor chip 820), and/or (ii) at least a portion of a surface of the second planar member 810.

In several embodiments, the plurality of spacer elements 815 comprise a material that has greater flexibility than one or both of the first planar member 805 and the second planar member 810; i.e., relatively speaking, one or both of the first planar member 805 and the second planar member 810 may be considered to be rigid relative to the plurality of spacer elements 815 and the plurality of spacer elements 815 may be considered to be flexible relative to one or both of the first planar member 805 and the second planar member 810. In other embodiments, the plurality of spacer elements 815 comprise a material that has less flexibility than one or both of the first planar member 805 and the second planar member 810; i.e., relatively speaking, one or both of the first planar member 805 and the second planar member 810 may be considered to be flexible relative to the plurality of spacer elements 815 and the plurality of spacer elements 815 may be considered to be rigid relative to one or both of the first planar member 805 and the second planar member 810.

In particular, it has been discovered that if the imaging chamber 800 is formed using spacer elements disposed between planar members, and if at least one the first planar member 805 and the second planar member 810, and/or the plurality of spacer elements 815 is flexible, the imaging chamber 800 behaves differently than the conventional hemocytometer chambers, and the difference is highly advantageous. When the imaging chamber 800 is filled with a liquid (e.g., a blood sample), the capillary forces tend to pull the first planar member 805 and the second planar member 810 together, thus exerting a slight pressure on the retained plurality of spacer elements 815. This pressure will cause the flexible element to deform in such a manner as to cause the chamber height (h1) to approximate, on average, the mean dimension of the plurality of spacer elements 815 (the average spacer height (h2)) disposed between the first planar member 805 and the second planar member 810. For example, if both the first planar member 805 and the second planar member 810 are rigid and the plurality of spacer elements 815 are flexible, separators larger than the mean diameter will be compressed, and the first planar member 805 and the second planar member 810 will approximate until more and more separators come into contact with the first planar member 805 and the second planar member 810, preventing further approximation. At that point, the height of the chamber (h1) will have an average height which substantially approximates the average height (h2) of the plurality of spacer elements 815 and is readily ascertainable, provided the standard deviation of the heights of the plurality of spacer elements 815 is acceptable and the plurality of spacer elements 815 are sufficiently flexible. As used herein, the term “flexibility” is the ability of a material to deform elastically and return to its original shape when the applied force or stress is removed (defined as the displacement caused by a unit force). As used herein, the terms “flexible” and “sufficiently flexible” are defined as an object having a Taber stiffness of less than 0.4 mN/m. As used herein, the terms “rigidity” and “stiffness” are the inverse of flexibility and is the extent to which a material capable resisting deformation upon the application of a force or stress (defined as the force required to produce a unit displacement). As used herein, the term “rigid”, “stiff”, “sufficiently rigid”, and “sufficiently stiff” are defined as an object having a Taber stiffness of greater than 0.4 mN/m. In another example, if the plurality of spacer elements 815 are rigid and the first planar member 805 and/or the second planar member 810 is flexible, the first planar member 805 and/or the second planar member 810 will deform and be “tented-up” in a small area around each of the larger separators and be lower over smaller separators. The height of the chamber (h1) will have an average height which substantially approximates the average height (h2) of the plurality of spacer elements 815, provided the first planar member 805 and/or the second planar member 810 is sufficiently flexible.

The height of the spacer elements can be determined by any known analytical technique typically used to measure the height or size of a structure, such as laser device, SEM imaging, internal standard means, etc. An example of an internal standard includes a flexible or flowable material which is not miscible with the sample and which contains a known, stable and uniform concentration of a sensible optical dye. The material can be dyed flexible beads, dyed oil or the like, and may be present in one or more areas of the chamber. Since the optical density is in direct proportion to the thickness of the calibrator material, measurement of the optical density of the part of the calibrator material, which completely fills the chamber height, will allow the calculation of the exact chamber height at a set location to within the precision capabilities of the optical system. As used herein, the “average chamber height” means the average height of at least 90% of the chamber. Average is understood as a calculated “central” value of a set of numbers (the sum of the set of numbers divided by the count), where the set of numbers is the set of height values for at least 90% of the chamber.

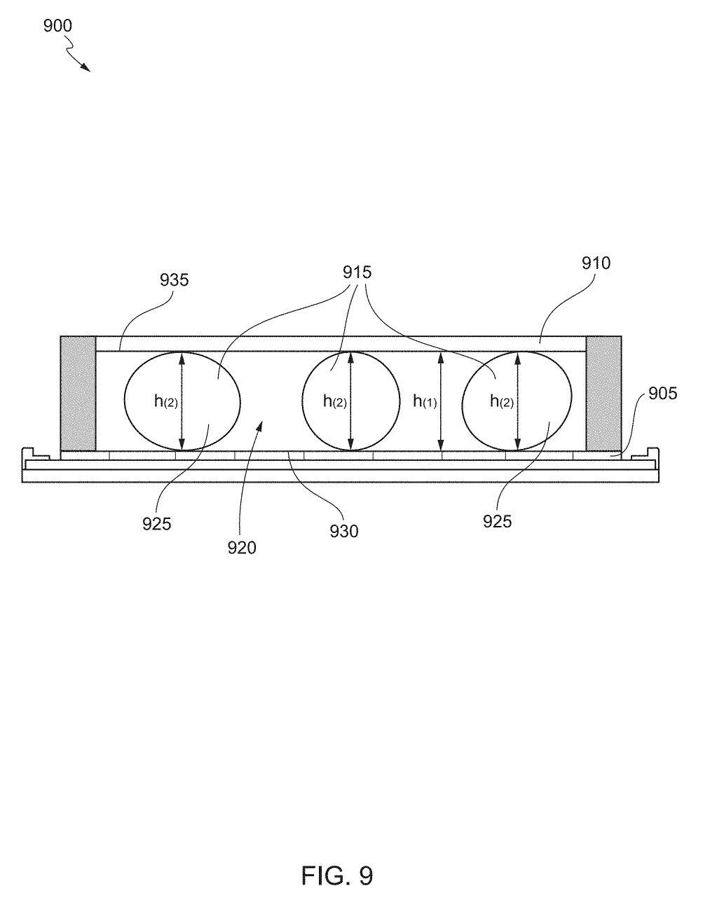

As shown in FIG. 9, in some embodiments, an imaging device 900 comprises a first planar member 905 and a second planar member 910 separated by a plurality of spacer elements 915 that define an imaging chamber 920 between the first planar member 905 and the second planar member 910. The plurality of spacer elements 915 are formed from a material that has greater flexibility than the first planar member 905 and the second planar member 910; i.e., the first planar member 905 and the second planar member 910 may be considered to be rigid relative to the plurality of spacer elements 915 and the plurality of spacer elements 915 may be considered to be flexible relative to the first planar member 905 and the second planar member 910. In this example, larger spacer elements 925 may be compressed to the point where the first planar member 905 and the second planar member 910 have approximated to the point where most of the plurality of spacer elements 915 are touching the interior surfaces 930, 935 of the first planar member 905 and the second planar member 910, respectively, thereby making the average spacer height (h2) substantially equal to the chamber height (h1) when the blood sample is received in the sample testing conduit. Testing indicates that that the desired chamber height (h1) can be controlled to 1% or better at chamber heights of less than four microns.

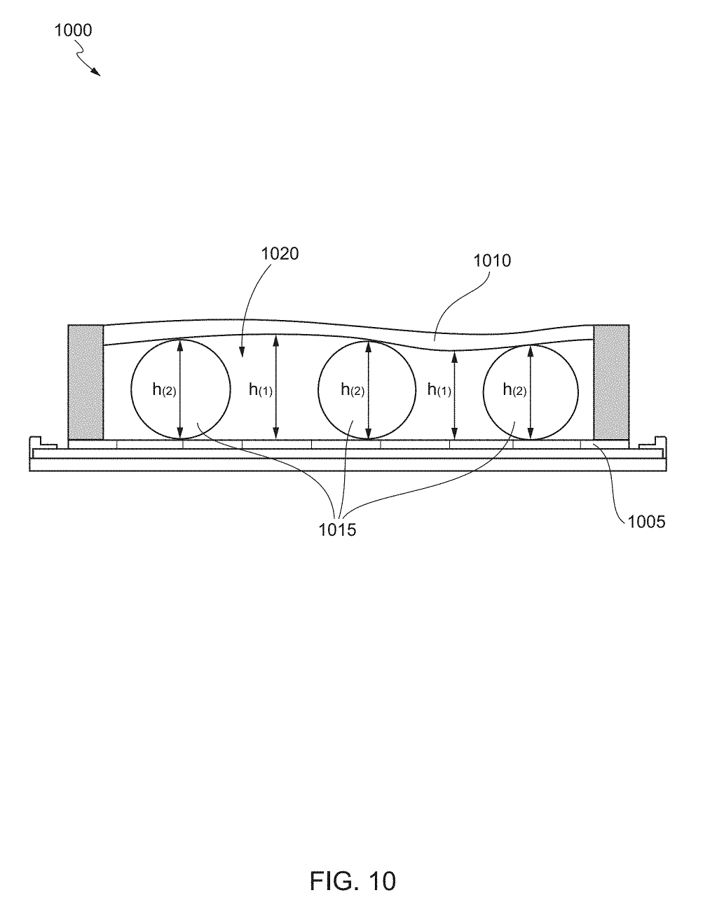

As shown in FIG. 10, in other embodiments, an imaging device 1000 comprises a first planar member 1005 and a second planar member 1010 separated by a plurality of spacer elements 1015 that define an imaging chamber 1020 between the first planar member 1005 and the second planar member 1010. The second planar member 1010 is formed from a material more flexible than the plurality of spacer elements 1015 and the first planar member 1005, and will overlay the plurality of spacer elements 1015 in a tent-like fashion i.e., relatively speaking, the second planar member 1010 may be considered to be flexible relative to the plurality of spacer elements 1015, and the plurality of spacer elements 1015 may be considered to be rigid relative to the second planar member 1010. In this example, it should be apparent although small local areas of the imaging chamber 1020 will deviate from the desired chamber height (h1), the average height of all the tented areas will approximate the average spacer height (h2), thereby making the average spacer height (h2) substantially equal to the chamber height (h1) when the blood sample is received in the sample testing conduit. Testing indicates that that the desired chamber height (h1) can be controlled to 1% or better at chamber heights of less than four microns.

### Imaging Assay Beads

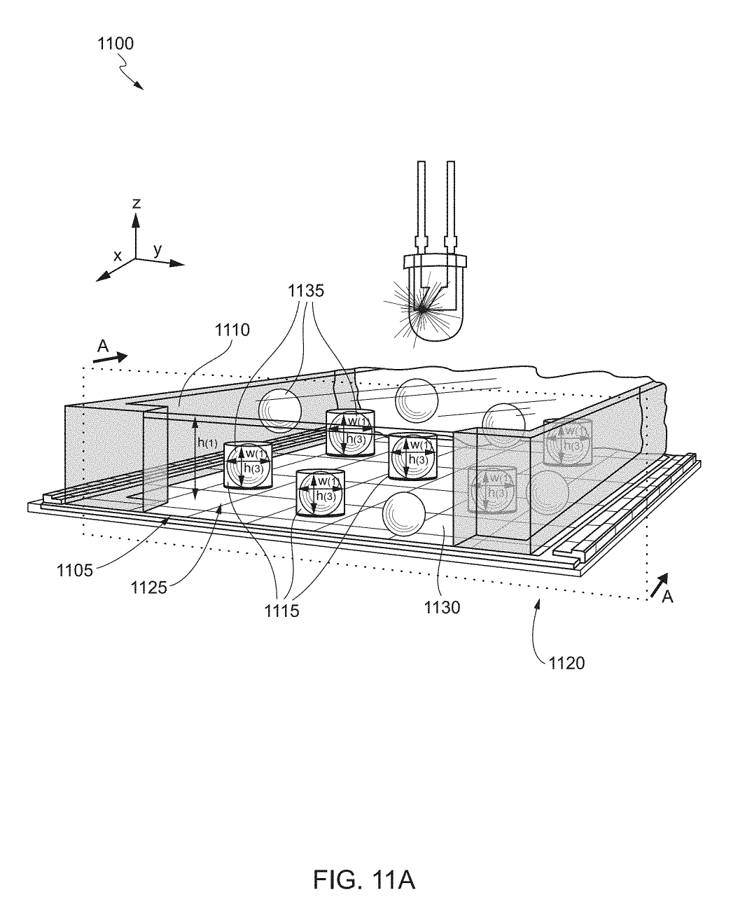

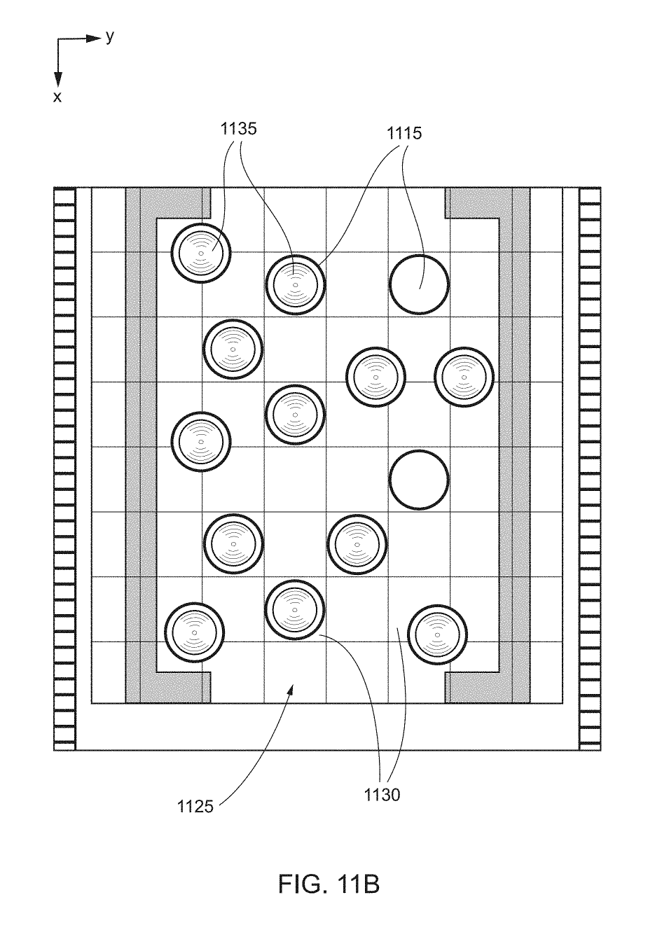

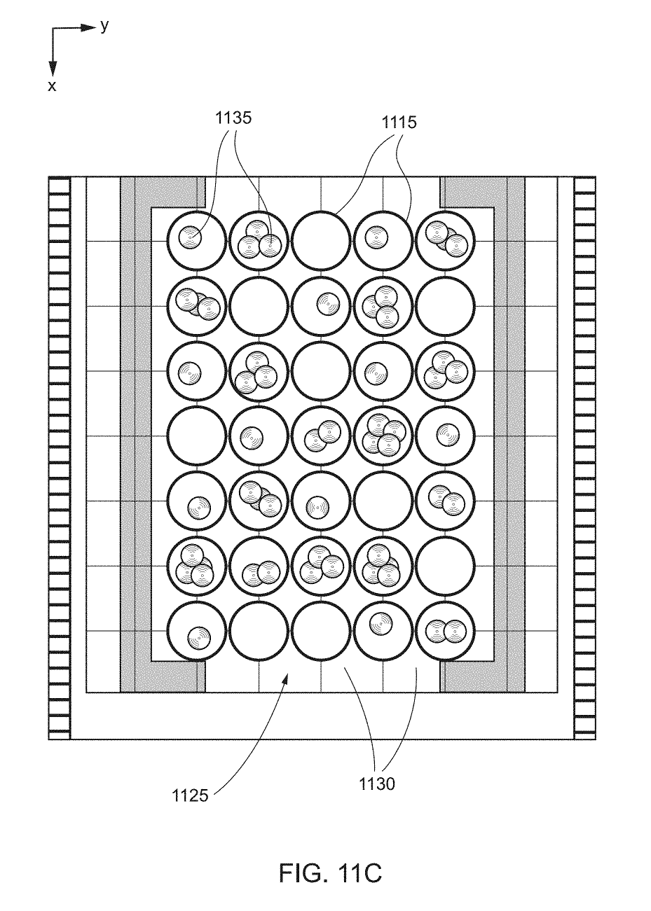

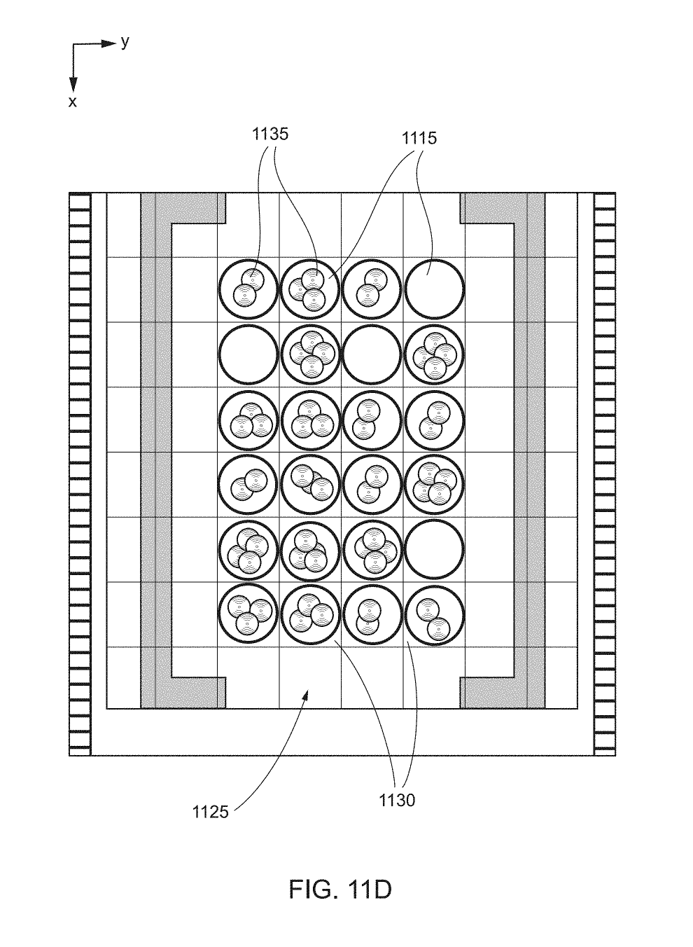

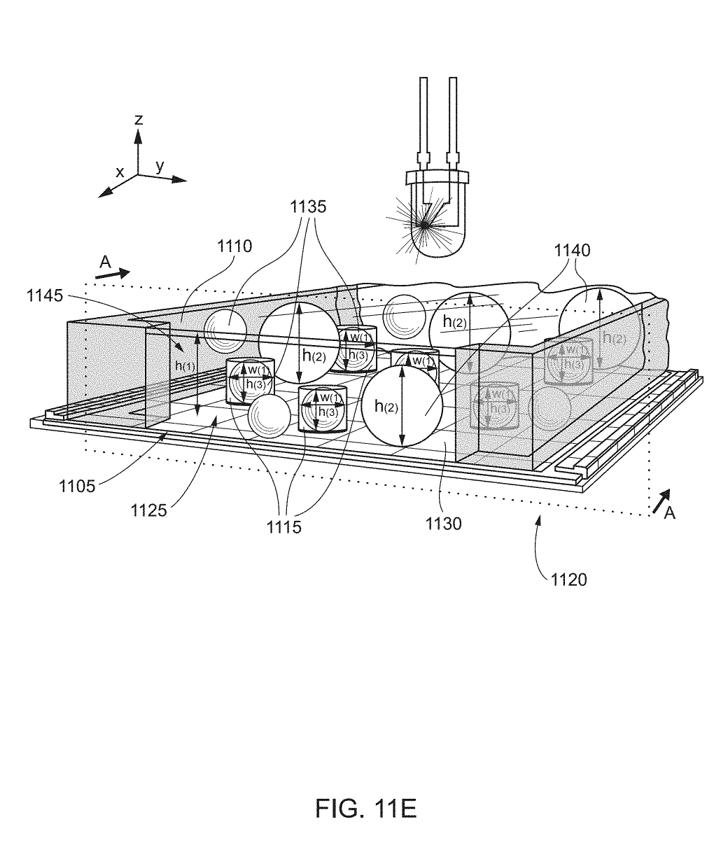

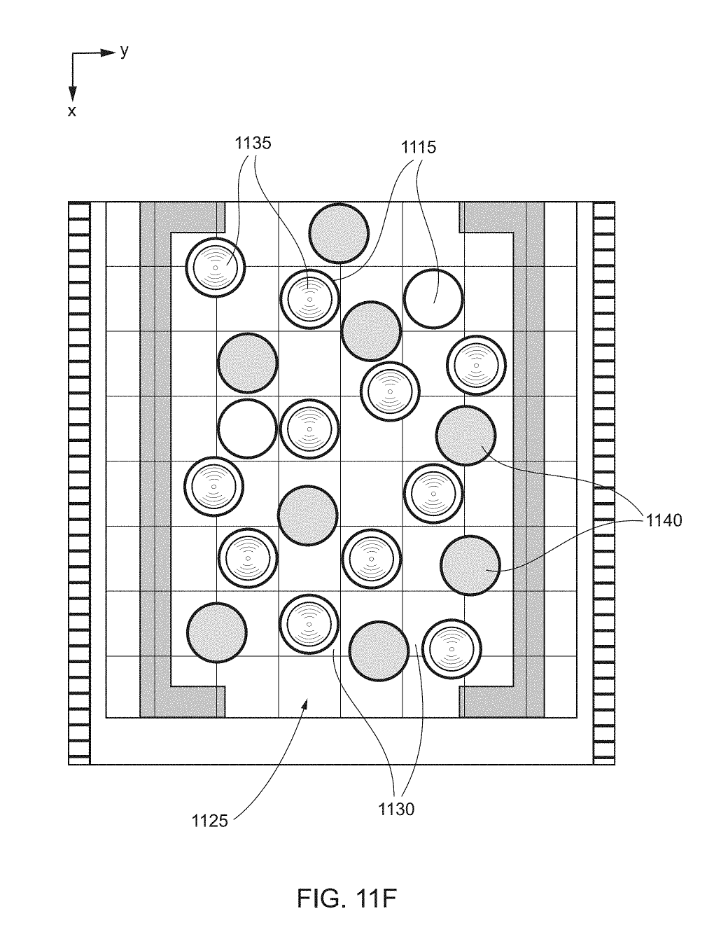

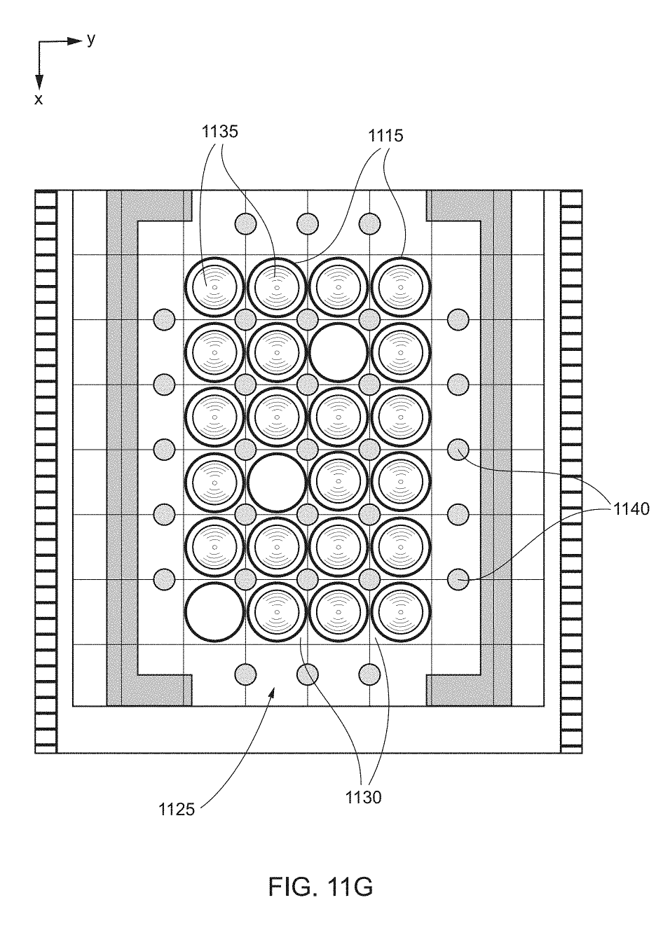

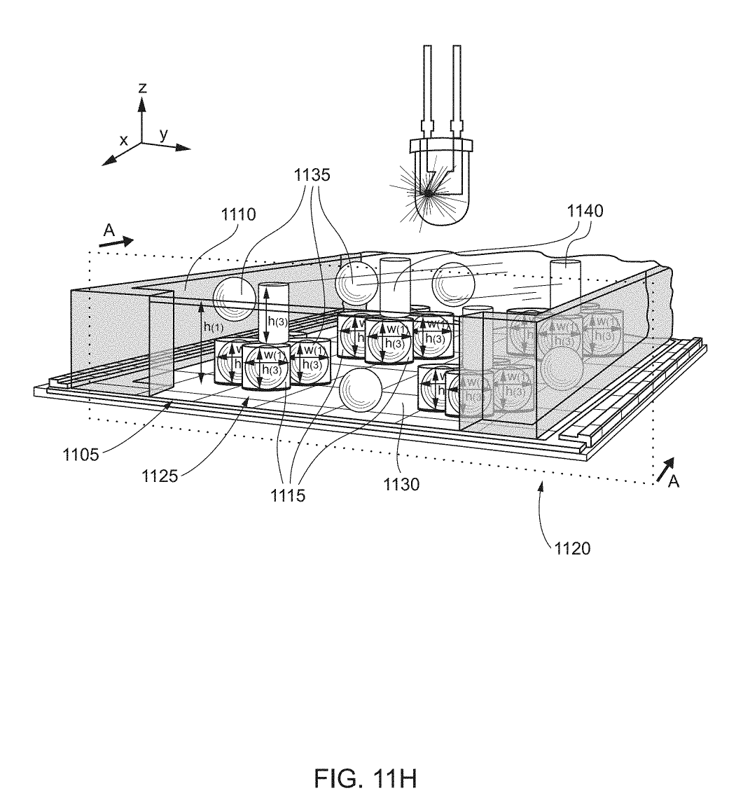

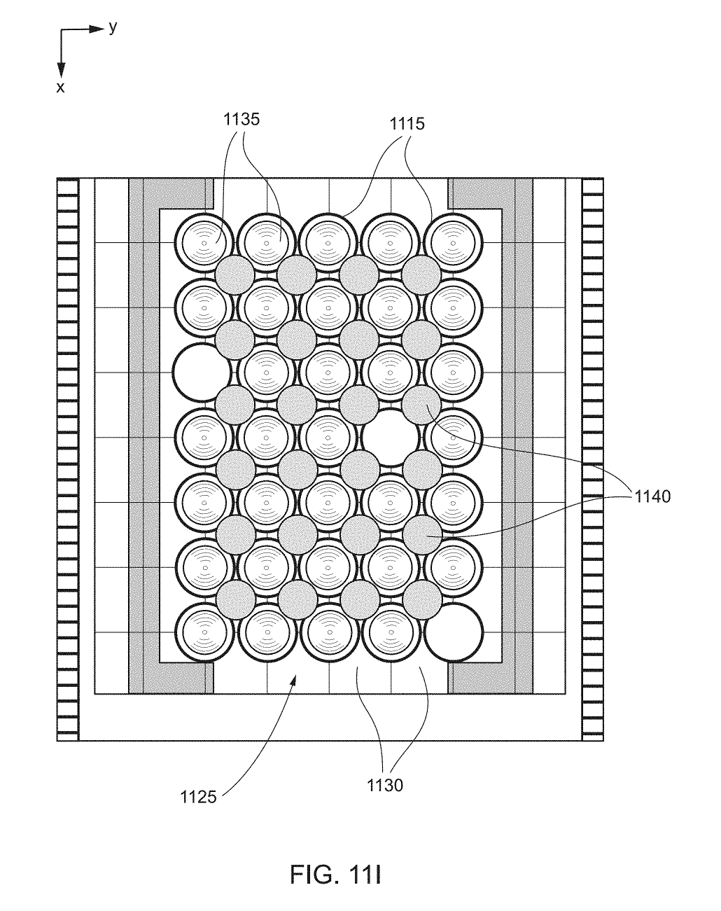

As shown in FIG. 11A, an imaging chamber 1100 may comprise a first planar member 1105, a second planar member 1110, and a plurality of wells 1115 disposed between the first planar member 1105 and the second planar member 1110. A height (h1) of the imaging chamber 1100 may be predetermined such that the sample residing within the imaging chamber 1100 will travel laterally within the imaging chamber 1100 via capillary forces. In various embodiments, at least one of the first planar member 1105 and the second planar member 1110 is transparent (e.g., a transparent planar member). Transparent plastic films comprising acrylic or polystyrene are examples of acceptable materials for the first planar member 1105 and the second planar member 1110. In some embodiments, at least a portion of the first planar member 1105 or the second planar member 1110 (e.g., a planar member) is formed from the imager chip 1120, as similarly described with respect to imaging chamber 800 illustrated in FIG. 8.

As shown in FIGS. 11B, 11C, and 11D, the plurality of wells 1115 may be arranged on the photosensitive surface 1125 in a pattern such that the wells are aligned vertically with one or more of the pixels 1130 in the array of pixels. In some embodiments, the plurality of wells 1115 are arranged on the photosensitive surface 1125 such that at least 50% of the wells are aligned vertically with one or more of the pixels 1130 in the array of pixels (see, e.g., FIG. 11B). In several embodiments, the plurality of wells 1115 are arranged on the photosensitive surface 1125 such that each of the wells is aligned vertically with one or more of the pixels 1130 in the array of pixels (see, e.g., FIG. 11C). In certain embodiments, the plurality of wells 1115 are arranged on the photosensitive surface 1125 such that each of the wells is aligned vertically with exactly one of the pixels in the array of pixels (see, e.g., FIG. 11D). Consequently, the alignment between the wells and the pixels facilitates satisfaction of the near-field criterion and resolution of the assay beads to be captured within the wells.

In various embodiments, the plurality of wells 1115 are any structure that is disposable between the first planar member 1105 and the second planar member 1110, and operable to hold one or more assay beads 1135. In certain embodiments, each well 1115 is sized and each assay bead 1135 is sized such that each well 1115 is structured to hold exactly one assay bead 1135 (see, e.g., FIG. 11B). In other embodiments, each well 1115 is sized and each assay bead 1135 is sized such that each well 1115 is structured to hold at least one assay bead 1135 (see, e.g., FIG. 11C). In other embodiments, each well 1115 is sized and each assay bead 1135 is sized such that each well 1115 is structured to hold a plurality of assay beads 1135 (see, e.g., FIG. 11D). The height of each of the plurality of wells 1115 typically do not equal one another exactly, but may be substantially equal. As such the height of the plurality of wells 1115 may be characterized as the average well height (h3). In some embodiments, the average well height (h3) is selected to accommodate the analysis of the assay beads 1135 and will be from about 0.5 μm to about 40 μm, from about 0.5 μm to about 20 μm, or from about 2 μm to about 10 μm, for example about 4 μm, about 8 μm, or about 10 μm. The width of each of the plurality of wells 1115 typically do not equal one another exactly, but may be substantially equal. As such the width of the plurality of wells 1115 may be characterized as the average well width (w1). In some embodiments, the average well width (w1) is selected to accommodate the analysis of assay beads 1135 and will be from about 0.5 μm to about 40 μm, from about 2.0 μm to about 20 μm, or from about 2 μm to about 10 μm, for example about 4 μm, about 10 μm, or about 15 μm.

In some embodiments, the plurality of wells 1115 are columns (e.g., round or square uniform polymer, silica or polystyrene columns). In some embodiments, the plurality of wells 1115 are hollow pillar structures fabricated on: (i) at least a portion of a surface of the first planar member 1105 (e.g., the photosensitive surface 1125 of the sensor chip 1120), and/or (ii) at least a portion of a surface of the second planar member 1110. In some embodiments, the plurality of wells 1115 are hollow structures embossed on: (i) at least a portion of a surface of the first planar member 1105 (e.g., the photosensitive surface 1125 of the sensor chip 1120), and/or (ii) at least a portion of a surface of the second planar member 1110. In some embodiments, the plurality of wells 1115 are formed directly (physical contact between the objects) on: (i) at least a portion of a surface of the first planar member 1105 (e.g., the photosensitive surface 1125 of the sensor chip 1120), and/or (ii) at least a portion of a surface of the second planar member 1110. In some embodiments, the plurality of spacer elements 1115 are formed indirectly (no physical contact between the objects) on: (i) at least a portion of a surface of the first planar member 1105 (e.g., the photosensitive surface 1125 of the sensor chip 1120), and/or (ii) at least a portion of a surface of the second planar member 1110.

In various embodiments, the assay beads 1135 are microparticles having a general shape such as spherical, cylinder, cube, dodecahedron, elliptical, or other regular or irregular shapes. In some embodiments, the assay beads 1135 are formed of a polymer such as a latex, glass, silica, or polystyrene. In other embodiments, the assay beads 1135 are formed of a magnetic material such that they exhibit magnetic properties when placed in a magnetic field with no residual magnetism once removed from the magnetic field. The assay beads 1135 may have a diameter, width and/or length from about 0.1 μm to about 35 μm, from about 0.1 μm to about 20 μm, or from about 0.1 μm to about 10 μm. The assay beads 1135 may be coated with a reagent capable of binding a target antigen in a sample. The reagent may comprise an antibody, an antibody fragment, an ionophore, an enzyme, a set of enzymes, a peptide with a cleavable detectable moiety, an optical marker dye identifying a type of assay bead, and/or combinations thereof.

In some embodiments, the assay beads 1135 are mobile within the imagining chamber 1100, which can accelerate binding reactions, making the capture step of the assay faster. For example, the imaging chamber 1100 can contain mobile assay beads 1135 capable of interacting with an analyte and being localized over the photosensitive surface 1125, whereby motion of specimen via capillary forces, active forces (e.g., via a pump) and/or gravity in the imaging chamber 1100 is used to capture the mobile assay beads 1135 in the plurality of wells 1115. Alternatively, the imaging chamber 1100 can contain mobile magnetic assay beads 1135 capable of interacting with an analyte and being localized over the photosensitive surface 1125, whereby magnetic forces are used to capture the mobile magnetic assay beads 1135 in the plurality of wells 1115. In some embodiments, exactly one assay bead 1135 is capable of being captured (by capillary action, active action, gravity or magnetic force) in each of the plurality of wells 1115; however, not all wells 1115 must capture a bead 1135 (see, e.g., FIG. 11B). For example, the plurality of wells 1115 are arranged such that at least 50% of the wells 1115 capture an assay bead 1135, at least 65% of the wells 1115 capture an assay bead 1135, or at least 85% of the wells 1115 capture an assay bead 1135. In other embodiments, at least one assay bead 1135 is capable of being captured (by capillary action, active action, gravity or magnetic force) in each of the plurality of wells 1115; however, not all wells 1115 must capture at least one assay bead 1135 (see, e.g., FIG. 11C). For example, the plurality of wells 1115 are arranged such that at least 60% of the wells 1115 capture at least one assay bead 1135, at least 75% of the wells 1115 capture at least one assay bead 1135, or at least 85% of the wells 1115 capture at least one assay bead 1135. In other embodiments, a plurality of assay beads 1135 are captured (by capillary action, active action, gravity or magnetic force) in each of the plurality of wells 1115; however, not all wells 1115 must capture a plurality of beads 1135 (see, e.g., FIG. 11D). For example, the plurality of wells 1115 are arranged such that at least 65% of the wells 1115 capture a plurality of assay beads 1135, at least 75% of the wells 1115 capture a plurality of assay beads 1135, or at least 85% of the wells 1115 capture a plurality of assay beads 1135.

In other embodiments, the assay beads 1135 are immobilized within the imagining chamber 1100. For example, the assay beads 1135 may be dispensed into the plurality of wells 1115, forming an adhered, porous bioactive layer. The bioactive layer has the property of binding specifically to the analyte of interest, or of manifesting a detectable change when the analyte is present, and is most preferably an immobilized antibody directed against a target analyte. In some embodiments, exactly one assay bead 1135 from the plurality of assay beads 1135 is immobilized in each of the plurality of wells 1115. In some embodiments, exactly one assay bead 1135 from the plurality of assay beads 1135 is immobilized in at least 50% of the plurality of wells 1115 (see, e.g., FIG. 11B). In other embodiments, at least one assay bead 1135 from the plurality of assay beads 1135 is immobilized in each of the plurality of wells 1115. In other embodiments, at least one assay bead 1135 from the plurality of assay beads 1135 is immobilized in at least 50% of the plurality of wells 1115 (see, e.g., FIG. 11C). In other embodiments, a plurality of assay beads 1135 from the plurality of assay beads 1135 are immobilized in a portion of each of the plurality of wells. In other embodiments, a plurality of assay beads 1135 from the plurality of assay beads 1135 are immobilized in a portion of at least 50% of the plurality of wells (see, e.g., FIG. 11D).

As shown in FIGS. 11E, 11F, 11G, and 11H, in various embodiments, the imaging chamber 1100 further comprises plurality of spacer elements 1140. The plurality of spacer elements 1140 may be any structure that is disposable between the first planar member 1105 and the second planar member 1110, and operable to space the first planar member 1105 and the second planar member 1110 apart from one another and maintain the chamber height (h1), as described with respect to the plurality of spacer elements 815 illustrated in FIG. 8. In some embodiments, the plurality of spacer elements 1140 have a predetermined average spacer height (h2) and are disposed between the first planar member 1105 and the second planar member 1115 to form a chamber 1145 having a predetermined average chamber height (h1) extending between the first planar member 1105 and the second planar member 1115. FIGS. 11E and 11F show that the plurality of spacer elements 1140 maybe spherical beads. FIG. 11G shows the plurality of spacer elements 1140 may be pillar structures fabricated on: (i) at least a portion of a surface of the first planar member 1105 (e.g., the photosensitive surface 1125), and/or (ii) at least a portion of a surface of the second planar member 1110. In several embodiments, the plurality of spacer elements 1140 comprise a material that has greater flexibility than one or both of the first planar member 1105 and the second planar member 1110. In other embodiments, the plurality of spacer elements 1140 comprise a material that has less flexibility than one or both of the first planar member 1105 and the second planar member 1110. For example, at least one of: (i) the second planar member 1110 and (ii) the plurality of spacer elements 1135, may be deformable such that the predetermined average spacer height (h2) is substantially equal to the predetermined average chamber height (h1) (e.g., (h2)=(h1)). Alternatively, at least one of: (i) the second planar member 1110 and (ii) the plurality of spacer elements 1135, may be rigid such that the predetermined average spacer height (h2) is substantially equal to the predetermined average chamber height (h1) (e.g., (h2)=(h1)).

In other embodiments, the plurality of spacer elements 1140 are disposable between the first planar member 1105 and the second planar member 1110 and disposable above or below the wells 1115 such that the spacer elements 1140 and the wells 1115 are operable together to space the first planar member 1105 and the second planar member 1110 apart from one another and maintain the chamber height (h1). FIGS. 11H and 11I show the plurality of spacer elements 1140 may be pillar structures fabricated on: (i) at least a portion of a surface of one or more wells 1115, and/or (ii) at least a portion of a surface of the second planar member 1110. For example at least one of: (i) the second planar member 1110 and (ii) the plurality of spacer elements 1135, is deformable such that the predetermined average well height (h3) in addition to the predetermined average spacer height (h2) is substantially equal to the predetermined average chamber height (h1) (e.g., (h3)+(h2)=(h1)) when the blood sample is received in the sample testing conduit. Alternatively, at least one of: (i) the second planar member 1110 and (ii) the plurality of spacer elements 1135, is rigid such that the predetermined average well height (h3) in addition to the predetermined average spacer height (h2) is substantially equal to the predetermined average chamber height (h1) (e.g., (h3)+(h2)=(h1)) when the blood sample is received in the sample testing conduit.

### Sensor and Chip Designs

In one embodiment, a microfabricated sensor chip (e.g., the at least one sensor chip 315 described with respect to FIG. 3) comprises a sensor or transducer (e.g., an optical sensor). In other embodiments, a microfabricated sensor chip comprises a sensor array including at least a first sensor (e.g., an electrochemical sensor) and a second sensor (e.g., an optical sensor). In some embodiments, the sensors are fabricated singularly, as adjacent structures within a sensor array of a single chip. In other embodiments, the sensors are fabricated singularly, separated from another within a sensor array of a plurality of chips, or any combination thereof. In certain embodiments, the microfabricated sensor chip(s) comprises a plastic, polyester, polyimide, or silicon planar substrate, a plastic, polyester, polyimide, or silicon non-planar substrate, a transparent plastic, polyester, polyimide, or silicon substrate, a printed circuit board (PCB), and the like.

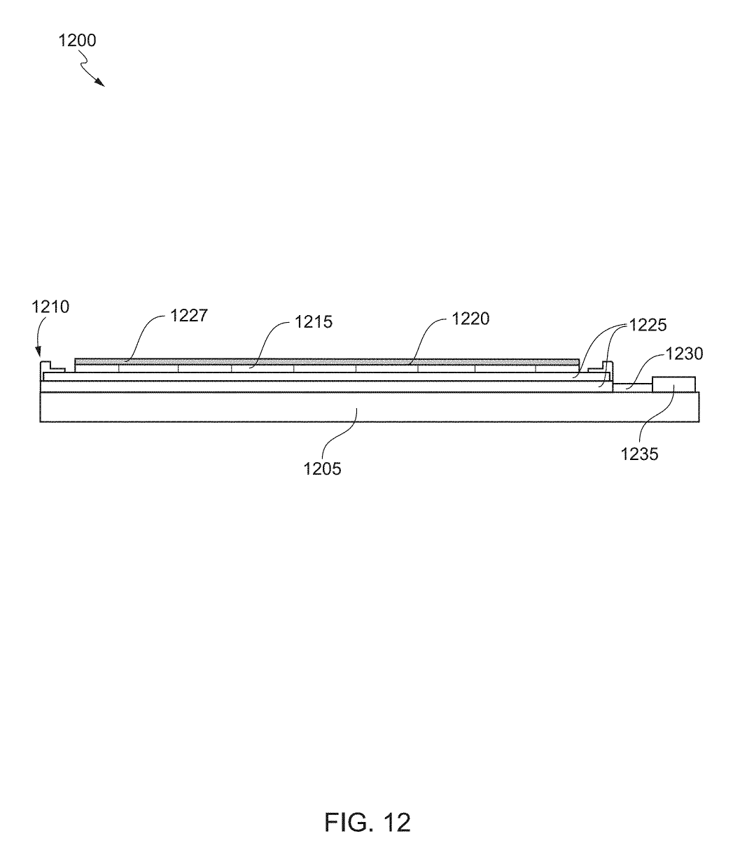

In various embodiments, the microfabricated sensor chip is an imager chip comprising one or more optical sensors such as a light detector. For example, wafer-level micro-fabrication of a preferred embodiment of an imager chip 1200 may be achieved as shown in FIG. 12. A non-conducting substrate 1205 having a planar top and bottom surface may be used as a base for the imager chip 1200. An imaging integrated circuit 1210 may be provided or formed on the substrate 1205 by conventional means, e.g., a micro-fabrication technique known to those of skill in the art to form a multi-dimensional array of pixels 1215 presented at its surface 1220 (i.e., a photosensitive surface) and non-photosensitive supporting circuitry 1225 for processing and readout. In certain embodiments in which a fluorescent dye such as acridine orange is used as a reagent, the imager chip 1200 further comprises a filter layer 1227 (e.g., an absorptive or dichroic filter layer) between the sample and the multi-dimensional array of pixels 1215. For example, the filter layer 1227 may be disposed on the top surface of the multi-dimensional array of pixels 1215. The filter layer 1227 is an optical filter layer which absorbs at a specific wavelength, or one or more bands of wavelengths, e.g. red, blue, green, etc. The filter layer 1227 may be manufactured on the imager chip 1200 as a coating, e.g., dispensed and dried to form the layer. Alternatively, the filter layer 1227 may be manufactured on the imager chip 1200 as patterned photoresist material as described for example in U.S. Pat. No. 5,200,051, which is incorporated herein by reference in its entirety. Alternatively, the filter layer 1227 may be manufactured on the imager chip 1200 as a separate film layer fixed to a surface of the imager chip 1200, e.g., the top surface of the multi-dimensional array of pixels 1215.

The microfabricated imager chip 1200 may further comprise an electrical connection 1230 (e.g., an electrical connection comprising a plurality of discrete contacts) that connects the imaging integrated circuit 1210 to one or more conductive pins 1235 such as a temporary electrical connector. The size of the multi-dimensional array of pixels 1215 may be selected to fit with other components (e.g., the conduits or sensor region) of the testing device, e.g., a multi-dimensional array of pixels 1215 may be provided as a surface mount diode (SMD) and chip scale packaging (CSP) may be used to fit a variety of testing devices. In some embodiments, the imager chip 1200 may have a width from about 1 mm to about 20 mm and a length from about 1 mm to about 20 mm (e.g., a width of about 5 mm and a length of about 6 mm), in order to accommodate a low profile multi-dimensional array of pixels 1215 that has the industry standard 2.0 mm×1.25 mm footprint, which provides high efficiency light detection and low power consumption. In accordance with various aspects of the present invention, the sensitivity of the multi-dimensional array of pixels 1215 may be within the range of 0.5 uA/cm²-4 uA/cm², for example substantially 1 uA/cm².

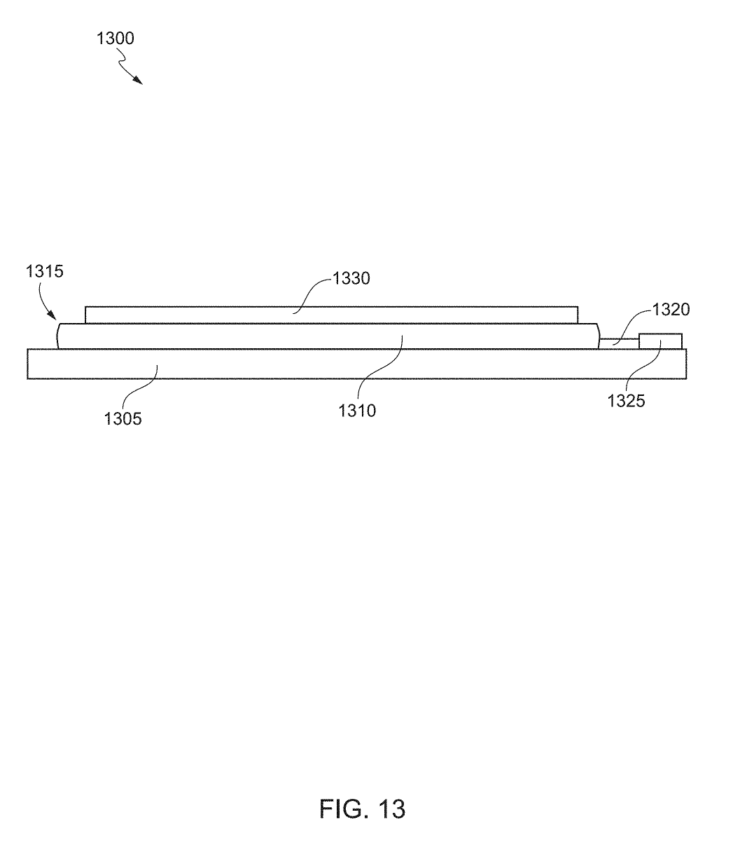

In various embodiments, the microfabricated sensor chip is an electrochemical sensor chip comprising one or more electrochemical sensors such as an amperometric electrode. For example, wafer-level micro-fabrication of a preferred embodiment of the electrochemical sensor chip 1300 may be achieved as shown in FIG. 13. A non-conducting substrate 1305 having a planar top and bottom surface may be used as a base for the electrochemical sensor chip 1300. A conducting layer 1310 may be deposited on the substrate 1305 by conventional means, e.g., screen printing, or micro-fabrication technique known to those of skill in the art to form at least one component 1315 (e.g., an amperometric electrode). The conducting layer 1310 may comprise a noble metal such as gold, platinum, silver, palladium, iridium, or alloys thereof, although other unreactive metals such as titanium and tungsten or alloys thereof may also be used, as many non-metallic electrodes of graphite, conductive polymer, or other materials may also be used. In certain embodiments, the one or more of the electrochemical sensors may be formed as electrodes with gold surfaces coated with a photo defined polyimide layer 1330 that includes openings to define a grid of small gold electrodes (e.g., a gold microarray electrode) at which an electroactive species may be oxidized. The electrochemical sensor chip 1300 may also comprise an electrical connection 1320 that connects each component of the conducting layer 1310 to one or more conductive pins 1325 such as a temporary (make and break) electrical connector.

In some embodiments, the electrochemical sensor chip 1300 comprises an array of 5-10 μm noble metal disks, e.g., 7 μm noble metal disks, on 15 μm centers. The array of noble metal disks or electrodes may cover a region, e.g., a circular region, approximately 300 to 900 μm in diameter, optionally 400-800 μm or about 600 μm in diameter, and may be formed by photo-patterning a thin layer of polyimide or photoresist of thickness up to 1.5 μm over a substrate made from a series of layers comprising Si,SiO2,TiW, and/or Au, or combinations thereof. In some embodiments, the electrodes have a working area of about 130,000 to 300,000 sq μm (i.e., a microelectrode), the volume of sample directly over the electrodes may be about 0.1-0.3 μL, and the volume of the sample over the sensor chip may be 1-3 μL. In accordance with these aspects of the present invention, the conduit (e.g., the conduit 465 described with respect to FIGS. 4A and 4D) in a region of the electrodes (e.g., the one or more sensor recesses 430 described with respect to FIGS. 4G- and 4H) has a volume to sensor area ratio of less than about 6 μL to about 1 square mm.

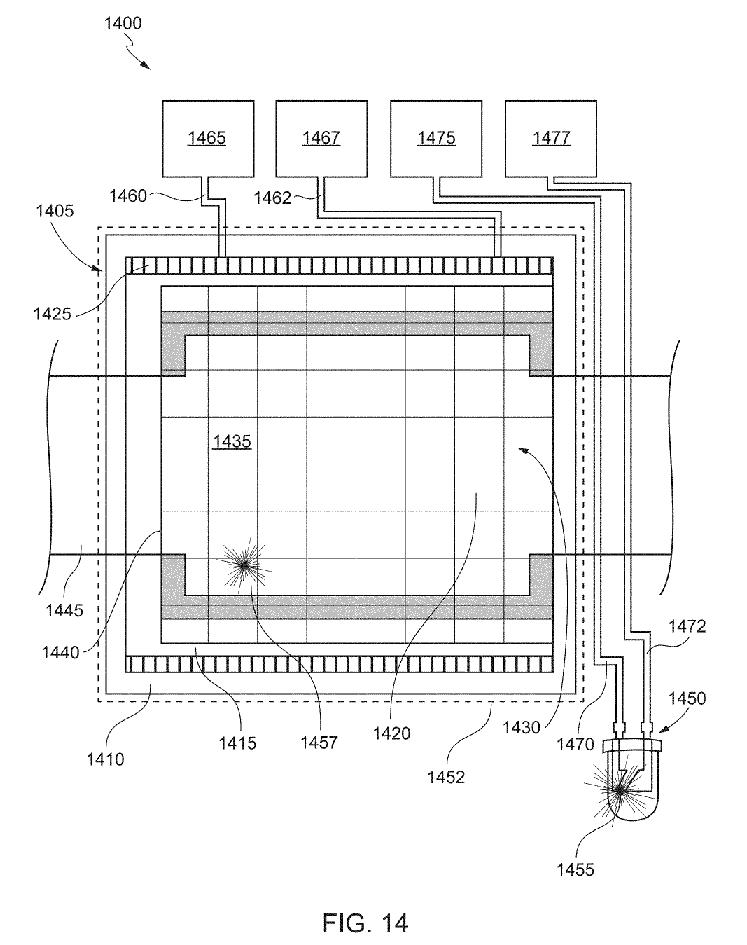

As shown in FIG. 14, in some embodiments, a microfabricated sensor chip 1400 includes an optical sensor 1405. The optical sensor 1405 may be formed of a non-conducting substrate 1410 and an imaging integrated circuit 1415 may be provided or formed on the substrate 1410. The imaging integrated circuit 1415 may include a multi-dimensional array of pixels 1420 for light detection and non-photosensitive supporting circuitry 1425 for processing and readout. The multi-dimensional array of pixels 1420 create a photosensitive surface 1430 of the optical sensor 1405. In certain embodiments, each pixel of the array of pixels 1420 comprises a photodetector (e.g., one or more light detectors) and optionally an amplifier in order to measure properties of light such as absorbance and florescence from an imaging chamber 1435 disposed adjacent to the optical sensor 1405. As described with respect to FIGS. 5-10 and 11A-11I, at least a portion of the photosensitive surface 1430 forms a portion of the wall or planar member 1440 of conduit 1445 (e.g., the conduit 465 described with respect to FIGS. 4A and 4D).

In various embodiments, one or more light emitters 1450 are positioned near the conduit 1445 (e.g., on a side of the conduit or above the conduit) and configured to transmit light through a portion of another wall or transparent planar member 1452 and the imaging chamber 1435 to the sensor chip 1400. In some embodiments, the one or more light emitters 1450 are located adjacent to the transparent planar member 1452 of the conduit 1445, and configured to transmit light through the transparent planar member 1452 and the imaging chamber 1435 to the sensor chip 1400. As such, the sensor chip 1400, the conduit 1445, and the one or more light emitters 1450 are contained within a same housing of the testing device (as described with respect to FIGS. 4A-4R). In other embodiments, the one or more light emitters 1450 are positioned near the transparent planar member 1452 of the conduit 1445 but not within same housing of the testing device, and instead are provided within the instrument or analyzer. As such, the sensor chip 1400 and the conduit 1445 are housed within the same housing (as described with respect to FIGS. 4A-4R), and the housing further comprises a window adjacent to the transparent planar member 1452 for illuminating the imaging chamber 1435 from the outside environment. In certain embodiments, the one or more light emitters 1450 are configured to transmit light through the window, the transparent planar member 1452, and the imaging chamber 1435 to the sensor chip 1400.

In several embodiments, the optical sensor 1405 is configured to measure the absorption of radiation (i.e., light), as a function of frequency or wavelength, due to the interaction of the radiation with a biological sample in the conduit 1445. In accordance with these aspects, the one or more light emitters 1450 are arranged to transmit incident light 1455 of one or more wavelengths through the conduit 1445 having the biological sample. Upon the incident light 1455 striking the sample, photons that match an energy gap of cells, a target analyte, or a chromatic substance related to a presence of the cells or the target analyte present in the biological specimen are absorbed. Other photons of light 1457 transmit through the conduit 1445 and biological specimen unaffected. The optical sensor 1405 is arranged to collect the photons of light 1457 transmitted through the conduit 1445 and the biological sample, and convert the transmitted photons of light 1457 into current. By comparing the attenuation of the photons of light 1457 with the incident light 1455, an absorption spectrum can be obtained to identify the presence, identity, count, and/or concentration of cells or the target analyte in the biological specimen.

In various embodiments, the optical sensor 1405 is connected via wirings 1460, 1462 to a conductive contact 1465, 1467, respectively (e.g., temporary electrical connector). In embodiments in which the one or more light emitters 1450 are provided within the same housing as the optical sensor 1405, the one or more light emitters 1450 are connected via wirings 1470, 1472 to another conductive contact 1475, 1477, respectively (e.g., temporary electrical connector). The wirings 1460, 1462, 1470, 1472 may be formed with gold surfaces that are optionally coated with a photo defined polyimide or photoresist layer such that the wirings 1460, 1462, 1470, 1472 are insulated from exposure to the environment of the sensor region (e.g., the biological sample disposed within the conduit 1445). The wirings 1460, 1462, 1470, 1472 terminate at the conductive contacts 1465, 1467, 1475, 1477, respectively (e.g., the discrete connector contacts 150 as described with respect to FIG. 1), which are used to make electrical contact with a connector (e.g., the multi-terminal connector 155 as described with respect to FIG. 1) in the analyzer (e.g., an i-STAT® cartridge reader as described in U.S. Pat. No. 4,954,087, the entirety of which is incorporated herein by reference). The design and arrangement of sensor chip 1400, optical sensor 1405, one or more light emitters 1450, wirings 1460, 1462, 1470, 1472, and/or conductive contacts 1465, 1467, 1475, 1477 is preferably selected based on printing and performance characteristics (e.g., minimize interference between multiple sensors, maximize transmission of light through the conduit and biological specimen, avoidance of interfering light, size constraints, etc.). However, it should be understood to those of ordinary skill in the art that any design or arrangement for the components is contemplated without departing from the spirit and scope of the present invention.

In some embodiments, the universal channel circuitry of the analyzer applies a drive current (e.g., a voltage greater than 2V and a current less than 1 mA), optionally via the conductive contact 1475, to the one or more light emitters 1450, and measures output current from the one or more light emitters 1450 via an optional conductive contact 1477. The output current is channeled from the conductive contact 1477 into the universal channel circuitry, and feedback resistor(s) of the universal channel circuitry may us the output current to set a nominal range of 0.5 mA to 4 mA, for example substantially 2 mA, which can provide over 1 mA at up to 4 V. The feedback resistor(s) are able to establish a constant current to continually drive the one or more light emitters 1450 for a predetermined period of time. The universal channel circuitry of the analyzer may control the optical sensor 1405 via the conductive contact 1467. The optical sensor 1405 channels output current (i.e., the current converted from the photons of light 1457 received from the one or more light emitters 1450) to the conductive contact 1465. The output current is channeled from the conductive contact 1465 into the universal channel circuitry and converted to a measurable voltage proportional to the amount of light detected by the optical sensor 1405. The processor (e.g., the processor 220 described with respect to FIG. 2) converts the measurable voltage to a qualitative, semi-quantitative, or quantitative value proportional to: (i) a number count or percentage for each type of cell in the blood sample, (ii) or an amount of target analyte in the biological specimen.

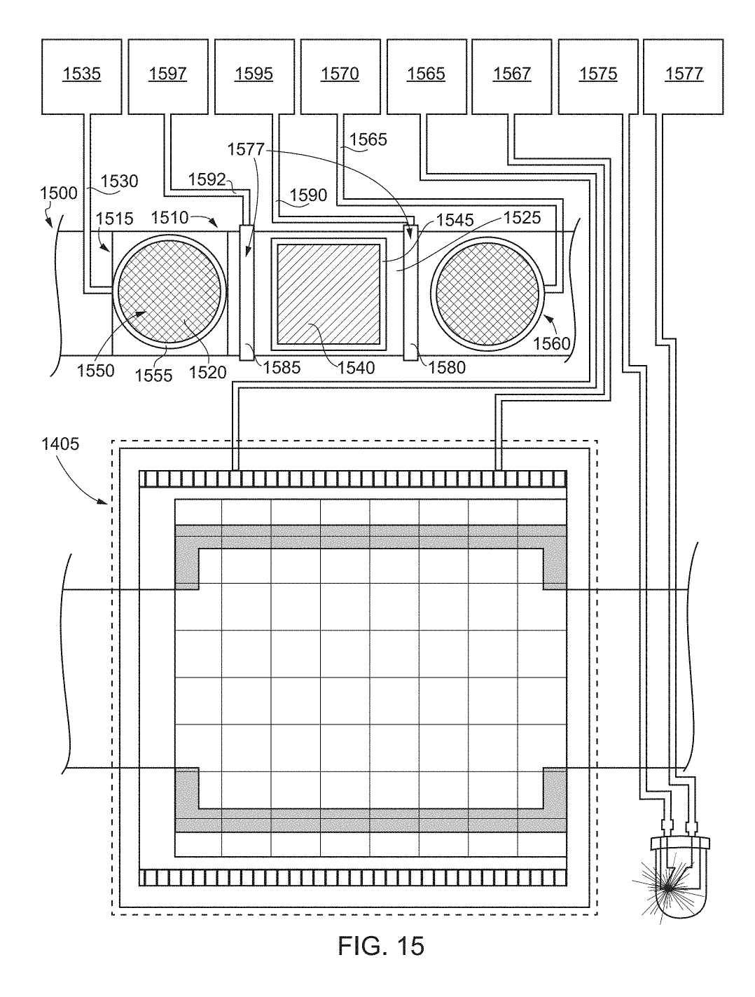

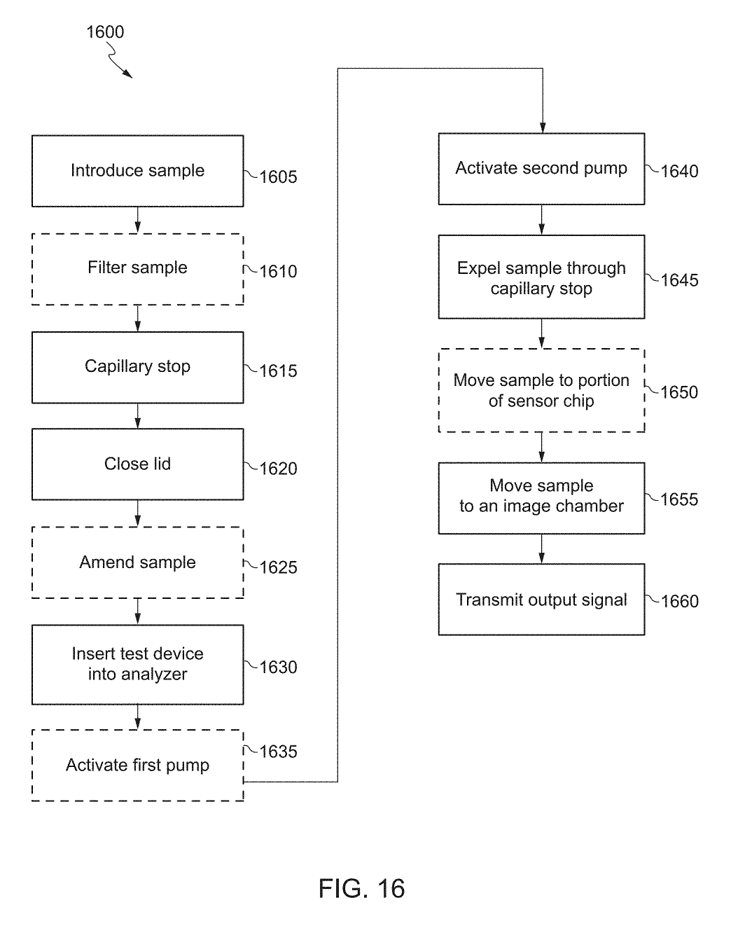

As shown in FIG. 15, in alternative embodiments, a microfabricated sensor chip 1500 includes a first sensor 1505 (e.g., an optical sensor) and optionally a different sensor chip 1510 or the same sensor chip 1500 includes a second sensor 1515 (e.g., an electrochemical sensor). The first sensor 1505 may be an optical sensor constructed as similarly described with respect to FIG. 14. The second sensor 1515 may be an electrochemical sensor constructed with an array of metal disks or electrodes that cover a region of the sensor chip 1500/1510. In some embodiments, the electrochemical sensor may be formed as electrodes 1520 with gold surfaces that are exposed (e.g., no polyimide or photoresist covering) to the inside environment of an auxiliary conduit 1525 and configured to directly contact the biological sample disposed within the conduit 1525. The second sensor 1515 may be connected via wiring 1530 to a conductive contact 1535 (e.g., temporary electrical connector). The wiring 1535 may be formed with a gold surface that is optionally coated with a photo defined polyimide or photoresist layer such that the wiring 1530 is insulated from exposure to the environment of the sensor region (e.g., the biological sample disposed within the conduit 1445). The wiring 1530 terminates at the conductive contact 1535 (e.g., the discrete connector contacts 150 as described with respect to FIG. 1), which are used to make electrical contact with a connector (e.g., the multi-terminal connector 155 as described with respect to FIG. 1) in the analyzer (e.g., an i-STAT® cartridge reader as described in U.S. Pat. No. 4,954,087, the entirety of which is incorporated herein by reference). The design and arrangement of sensor chips 1500/1510, first sensor 1505, second sensor 1515, wiring 1530, and/or conductive contact 1535 is preferably selected based on printing and performance characteristics (e.g., minimize interference between multiple sensors, maximize transmission of light through the conduit and biological specimen, avoidance of interfering light, size constraints, etc.). However, it should be understood to those of ordinary skill in the art that any design or arrangement for the components is contemplated without departing from the spirit and scope of the present invention.

In some embodiments, a portion of the sensor chip 1500/1510 (e.g., a top surface of the substrate), a wall of the auxiliary conduit 1525, and/or a wall of the sample receiving chamber (e.g., the sample receiving chamber 420 described with respect to FIGS. 4A-4R) can be coated with one or more dry reagents to amend the biological sample for an electrochemical assay. For example, the sensor chip 1500/1510 may include a reagent region 1540 coated with an antibody-enzyme conjugate for an analyte of interest. The reagent region 1540 may be defined by a containment ring structure 1545. In some embodiments, the containment ring structure 1545 is a hydrophobic ring of polyimide or another photolithographically produced layer. A microdroplet or several microdroplets (approximately 5-40 nL in size) or a series of about a 100 nanodroplets (approximately 50 to 1000 pL in size) containing the antibody-enzyme conjugate in some form may be dispensed or printed on the surface of the sensor chip 1500/1510. The photodefined ring structure 1545 contains this aqueous droplet allowing the reagent region 1540 to be localized to a precision of a few microns. The reagent region 1545 can be made from 0.03 to approximately 2 mm² in size. The upper end of this size is limited by the size of the conduit and sensor chip the sensor chip 1500/1510 in present embodiments, and is not a limitation of the invention.

The biological sample or a fluid may be passed at least once over the dry reagent, e.g., the reagent region 1540 to dissolve the reagent within the biological sample or fluid. Reagents used to amend biological samples or fluid within the cartridge may include the antibody-enzyme conjugate, magnetic beads coated with capture antibodies, or blocking agents that prevent either specific or non-specific binding reactions among assay compounds. Within a segment of the biological sample or fluid, the reagent can be preferentially dissolved and concentrated within a predetermined region of the segment. This is achieved through control of the position and movement of the segment. Thus, for example, if only a portion of a segment, such as the leading edge, is reciprocated over the reagent, then a high local concentration of the reagent can be achieved close to the leading edge. Alternatively, if a homogenous distribution of the reagent is desired, for example if a known concentration of a reagent is required for a quantitative analysis, then further reciprocation of the sample or fluid will result in mixing and an even distribution.

In certain embodiments, the second sensor 1515 is an immunosensor positioned in the auxiliary conduit 1525 for receiving a biological sample mixed with the antibody-enzyme conjugate that is configured to bind to a target analyte within the biological sample. For example, the second sensor 1515 may be configured to detect an enzymatically produced electroactive species (e.g., 4-aminophenol) from the reaction of a substrate (e.g., 4-aminophenylphosphate) with an antibody-enzyme conjugate (e.g., one or more antibodies bound to alkaline phosphatase (ALP)). In accordance with these aspects, the second sensor 1515 contains a capture region or regions 1550 coated with capture antibodies that are configured to bind to a target analyte bound to the antibody-enzyme conjugate. The capture region 1550 may be defined by a containment ring structure 1555. In some embodiments, the containment ring structure 1535 is a hydrophobic ring of polyimide or another photolithographically produced layer. A microdroplet or several microdroplets (approximately 5-40 nL in size) containing capture antibodies in some form, for example bound to beads or microspheres, may be dispensed on the surface of the second sensor 1515. The photodefined ring structure 1555 contains this aqueous droplet allowing the capture region 1550 to be localized to a precision of a few microns. The capture region 1550 can be made from 0.03 to approximately 2 mm² in size. The upper end of this size is limited by the size of the auxiliary conduit 1525 and sensor chip 1500/1510 in present embodiments, and is not a limitation of the invention.

In various embodiments, the microfabricated sensor array (e.g., a sensor array on the microfabricated sensor chip 1500, the different sensor chip 1510, or a completely different sensor chip such as a ground chip) further comprises a reference sensor or electrode 1560. In accordance with certain aspects, in which the second sensor 1515 is an amperometric sensors, the reference electrode 1560 is configured as a counter electrode to complete the circuitry. In a preferred embodiment, the reference electrode 1560 may comprise silver metal (Ag) and its silver salt (AgCl) deposited on a solid substrate (i.e., an Ag/AgCl reference electrode). The reference electrode 1560 may be connected via wiring 1565 to a reference contact 1570 (e.g., temporary electrical connector). The microfabricated sensor array may be designed such that the ground chip is positioned upstream of the semiconductor chip 1500/1510. However, it should be understood that other arrangements for sensor and ground chips are possible without departing from the spirit and scope of the present invention. For example, the sensor array may further comprise one or more additional sensor chips (not shown) configured to detect various analytes of potential interest, such as troponin I, troponin T, CKMB, procalcitonin, bHCG, HCG, NTproBNP, proBNP, BNP, myoglobin, parathyroid hormone, d-dimer, NGAL, galectin-3, and/or PSA, among other analytes.

In certain embodiments, the universal channel circuitry of the analyzer applies a potential via the conductive contact 1535 to the second sensor 1515 and the reference electrode 1560, and measures current changes generated by oxidation current from the substrate as an electrochemical signal. The electrochemical signal being proportional to the concentration of the analyte in the biological sample. The second sensor 1515 may have an applied potential of approximately +0 mV to 90 mV, e.g., 60 mV versus the reference electrode 1560 and, in another embodiment, the second sensor 1515 has an applied potential of approximately +40 mV versus the reference electrode 1560. The signal generated by the enzyme reaction product at approximately +10 mV is distinguishable from the signal generated by the unreacted substrate at approximately +200 mV. It should be noted that the exact voltages used to amperometrically detect the substrate and the analyte will vary depending on the chemical structure of the substrate. It is important that the difference in the voltages used to detect the substrate be great enough to prevent interference between the readings.

In various embodiments, the sensor chip 1500/1510 further includes one or more conductometric sensors 1577 (e.g., hematocrit sensors). The one or more conductometric sensors 1577 are configured to determine biological sample arrival and/or departure at the reagent region 1540 and biological sample arrival and/or departure at the first and second sensors 1505 and 1515. More specifically, the one or more conductometric sensors 1577 lie perpendicular to a length of the conduit 1525 or sensor conduit, and an electrical resistance between pairs of electrodes for the sensor 1577 may be used to monitor a relative position of a fluid front of the biological sample. For example, at the extremes, an open circuit reading may indicate that the biological sample has been pushed off the reagent region 1540 and a closed circuit reading may indicate the reagent region 1540 is covered with the biological sample.

In some embodiments, the one or more conductometric sensors 1577 comprise at least two electrodes 1580 and 1585 (i.e., electrode pair). The electrode 1580 may be positioned upstream from the reagent region 1540, and the electrode 1585 may be position downstream of the reagent region 1540 and upstream of the second sensor 1515. As shown in FIG. 15, the electrodes 1580 and 1585 may be connected via wirings 1590 and 1592 to a conductive contact 1595, which functions as a conductometric low pin, and a conductive contact 1597, which functions an alternating current source or conductometric high pin, respectively. The wirings 1590 and 1592 may be formed with a gold surface that is coated with a photo defined polyimide or photoresist layer such that the wirings 1590 and 1592 are insulated from exposure to the biological sample disposed within the conduits. As such, in some embodiments, the biological sample or fluid reaches the reagent region 1540 after departing the sample receiving chamber and passing over the electrode 1580, then the biological sample subsequently arrives at the second sensor 1515 after departing the reagent region 1540 and passing over the electrode 1585.

While some embodiments are disclosed herein with respect to certain types of sensors (e.g., optical, electrochemical, and conductometric sensors) being electrically connected to certain pins, this is not intended to be restrictive. Instead, it should be understood to those of ordinary skill in the art that any design or arrangement for the sensors and pins is contemplated without departing from the spirit and scope of the present invention. For example, the universal channel circuitry is configured in such a manner that any pin and connector connection can be used as a channel for optical, amperometric, conductometric, and/or potentiometric measurements.

### Assay Methods

FIGS. 16-22 show exemplary flowcharts for performing the process steps of the present invention. The steps of FIGS. 16-22 may be implemented using the computing devices and systems described above with respect to FIGS. 1-15. Specifically, the flowcharts in FIGS. 16-22 illustrate the architecture, functionality, and operation of possible implementations of the systems, methods and computer program products according to several embodiments of the present invention. In this regard, each block in the flowcharts may represent a module, segment, or portion of code, which comprises one or more executable instructions stored on non-transitory machine readable storage medium that when executed by one or more processors (e.g., a processor of the analyzer) cause the one or more processors to perform the specified logical function(s) within the one or more executable instructions. It should also be noted that, in some alternative implementations, the functions noted in the blocks may occur out of the order noted in the figure. For example, two blocks shown in succession may, in fact, be executed substantially concurrently, or the blocks may sometimes be executed in the reverse order, depending upon the functionality involved. It will also be noted that each block of the flowchart illustrations, and combinations of blocks in the flowchart illustrations, can be implemented by special purpose hardware-based systems that perform the specified functions or acts, or combinations of special purpose hardware and computer instructions.

FIG. 16 illustrates a method 1600 (with reference to the testing device 400 as illustrated in FIGS. 4A-4J) of using a testing device to perform an optical assay in accordance with one embodiment of the invention. At step 1605, an unmetered biological sample may be introduced into a sample receiving chamber (e.g., the sample receiving chamber 420 described with respect to FIGS. 4G and 4H) of a testing device, through a sample entry port (e.g., sealable sample entry port 415 described with respect to FIGS. 4B and 4C). Optionally at step 1610, the biological sample may be filtered to remove cells ( ) such that only a plasma fraction of the sample reaches the sensors (e.g., in some embodiments (e.g., assays performed using the assay beads), if the cells are not substantially removed they may scatter the light from the LED and affect assay performance). In some embodiments, the sample receiving chamber comprises the filter material such that only the plasma fraction reaches the sample metering portion of the device. In other embodiments, a first conduit (e.g., conduit 465 described with respect to FIG. 4A) comprises the filter material such that the metered portion of the sample is filtered to remove the cells. At step 1615, a capillary stop (e.g., capillary stop 422 described with respect to FIGS. 4G and 4H) may prevent passage of the sample into the first conduit (e.g., conduit 465 described with respect to FIG. 4A) at this stage, and the sample chamber is filled with the sample. The capillary stop at the end of the sample chamber delimits a metered portion of the biological sample. At step 1620, a lid (e.g., closable sealing member 417 described with respect to FIGS. 4A and 4B) maybe closed to prevent leakage of the biological sample from the sensing device. While the biological sample is within sample chamber or the first conduit, the biological sample may be optionally amended at step 1625 with a compound or compounds (e.g., reagents such as dyes, enzymes, enzyme substrate, activators, stabilizers, binders, anticoagulants, buffers, enzyme-labeled antibody conjugate and the like) present initially as a dry coating on the inner surface of the sample chamber or first conduit.

At step 1630, the testing device may be inserted into an analyzer (e.g., analyzer 105 described with respect to FIG. 1) in accordance with some aspects of the present invention. Optionally at step 1635, insertion of the sensing device into the analyzer may activate a first pump (e.g., the portion of the flexible zone 490 as described with respect to FIGS. 4A and 4B) or mechanism that punctures a fluid-containing package when the package is pressed against a spike (e.g., spike 442 as described with respect to FIGS. 4G and 4H). Fluid (e.g., a substrate) may thereby be expelled into a second conduit (e.g., conduit 437 as described with respect to FIGS. 4G and 4H) that is in fluidic communication with the first conduit. A constriction in the second conduit prevents further movement of the fluid. At step 1640, operation of a second pump (e.g., displaceable membrane 445 as described with respect to FIGS. 4A, 4B, 4G, and 4H) by the analyzer applies pressure to an air-bladder of the sensing device, forcing air through a third conduit (e.g., conduit 455 as described with respect to FIGS. 4G and 4H) and into the sample chamber at a predetermined location.

At step 1645, the metered portion of the biological sample is expelled through the capillary stop by air pressure produced within the air-bladder at step 1640 into the first conduit. Optionally at step 1650, the biological sample is moved forward within the first conduit to a portion of the first conduit (e.g., conduit 465 as described with respect to FIG. 4A) that is exposed to a sensor chip (e.g., sensor chip 1400 as described with respect to FIG. 14) by air pressure produced within the air-bladder such that the biological specimen can be amended with a compound or compounds (e.g., reagents such as enzymes, enzyme substrate, activators, stabilizers, buffers, enzyme-labeled antibody conjugate and the like) present initially as a dry coating on a portion of the sensor chip (i.e., one or more reagent regions). Additionally or alternatively, the fluid in the second conduit may be moved past the constriction into the first conduit and into contact with the biological specimen by air pressure produced by the first pump. The fluid may include a substrate that may be acted upon by the biological specimen and/or amended compounds to produce a chromatic substance. To facilitate the dissolution of the substrate, compound or compounds in the biological sample and/or promote efficient reaction, the biological sample may be oscillated by air pressure produced within the air-bladder. In one embodiment, an oscillation frequency of between about 0.2 Hz and about 5 Hz is used, most preferably about 0.7 Hz.

At step 1655, the biological sample is move forward within the first conduit to a portion of the first conduit forming an imaging chamber (e.g., imaging chamber 530 as described with respect to FIG. 5) that is exposed to the sensor chip (e.g., sensor chip 505 or 1400 as described with respect to FIGS. 5 and 14) by air pressure produced within the air-bladder such that analysis (e.g., optical analysis) of the biological specimen can be performed. Once the biological sample is move forward to the imaging chamber, the biological specimen is dispersed through-out the imaging chamber by capillary action. In various embodiments, the biological sample dispersed though-out the imaging chamber over the photosensitive surface such that one or more light emitters can transmit incident light of one or more wavelengths into the portion of the imaging chamber and the biological specimen. Upon the incident light striking the biological sample, photons that match an energy gap of cells, a target analyte, or a chromatic substance related to a presence of cells or the target analyte present in the biological specimen are absorbed. Other photons transmit through the imaging chamber and biological specimen unaffected. The photosensitive surface collect the photons of light transmitted through the imaging chamber and the biological sample, and convert the transmitted photons of light into current. At step 1660, the current is transmitted to the analyzer as an output signal via a conductive contact, and the analyzer compares the attenuation of the transmitted light with the incident light to obtain an absorption spectrum and converts the output signal to: (i) to a number count or percentage for each type of cell in the blood sample, or (ii) an analyte signal proportional to the light received from the imaging chamber and collected by the photosensitive surface.

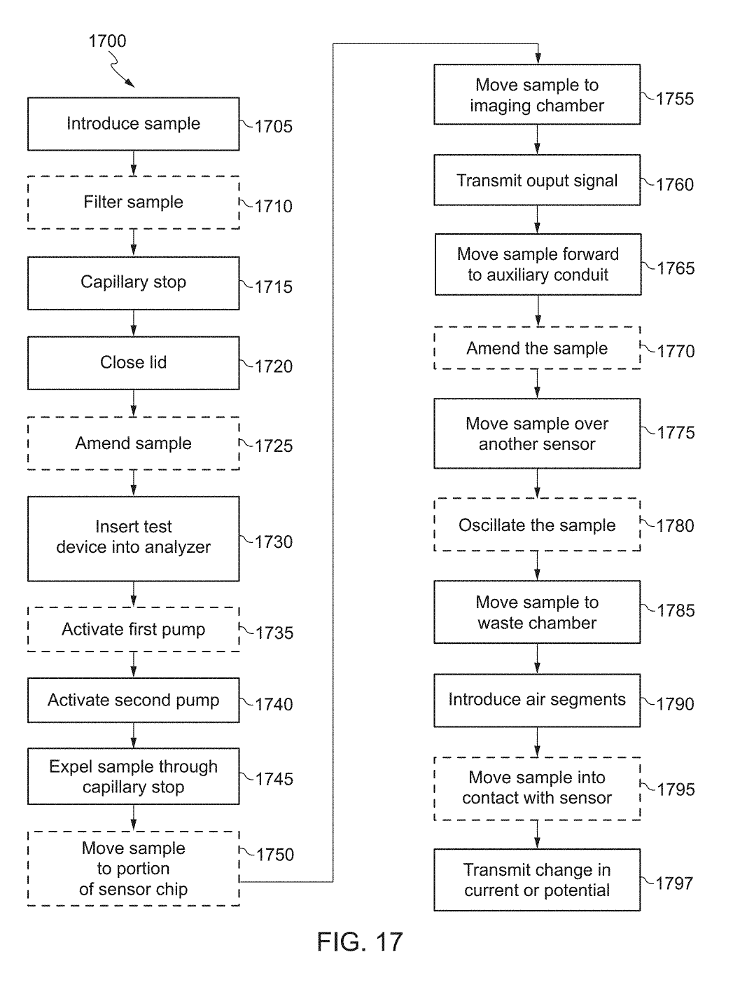

FIG. 17 illustrates a method 1700 (with reference to the testing device 400 as illustrated in FIGS. 4A-4J) of using a testing device to perform an optical assay and an electrochemical assay in accordance with one embodiment of the invention. At step 1705, an unmetered biological sample may be introduced into a sample chamber (e.g., the sample holding chamber 420 described with respect to FIGS. 4G and 4H) of a testing device, through a sample entry port (e.g., sealable sample entry port 415 described with respect to FIGS. 4B and 4C). Optionally at step 1710, the biological sample may be filtered to remove cells such that only a plasma fraction of the sample reaches the sensors (e.g., in some embodiments, if the cells are not substantially removed they may scatter the light from the LED and affect assay performance). In some embodiments, the sample receiving chamber comprises the filter material such that only the plasma fraction reaches the sample metering portion of the device. In other embodiments, a first conduit (e.g., conduit 465 described with respect to FIG. 4A) comprises the filter material such that the metered portion of the sample is filtered to remove the cells. At step 1715, a capillary stop (e.g., capillary stop 422 described with respect to FIGS. 4G and 4H) may prevent passage of the sample into the first conduit (e.g., conduit 465 described with respect to FIG. 4A) at this stage, and the sample chamber is filled with the sample. The capillary stop at the end of the sample chamber delimits a metered portion of the biological sample. At step 1720, a lid (e.g., closable sealing member 417 described with respect to FIGS. 4A and 4B) maybe closed to prevent leakage of the biological sample from the sensing device. While the biological sample is within sample chamber or the first conduit, the biological sample may be optionally amended at step 1725 with a compound or compounds (e.g., reagents such as dyes, enzymes, enzyme substrate, activators, stabilizers, binders, anticoagulants, buffers, enzyme-labeled antibody conjugate and the like) present initially as a dry coating on the inner surface of the sample chamber or first conduit.

At step 1730, the testing device may be inserted into an analyzer (e.g., analyzer 105 described with respect to FIG. 1) in accordance with some aspects of the present invention. Optionally at step 1735, insertion of the sensing device into the analyzer may activate a first pump (e.g., the portion of the flexible zone 490 as described with respect to FIGS. 4A and 4B) or mechanism that punctures a fluid-containing package when the package is pressed against a spike (e.g., spike 442 as described with respect to FIGS. 4G and 4H). Fluid (e.g., a substrate) may thereby be expelled into a second conduit (e.g., conduit 437 as described with respect to FIGS. 4G and 4H) that is in fluidic communication with the first conduit and/or an auxiliary conduit. A constriction in the second conduit prevents further movement of the fluid. At step 1740, operation of a second pump (e.g., displaceable membrane 445 as described with respect to FIGS. 4A, 4B, 4G, and 4H) by the analyzer applies pressure to an air-bladder of the sensing device, forcing air through a third conduit (e.g., conduit 455 as described with respect to FIGS. 4G and 4H) and into the sample chamber at a predetermined location.

At step 1745, the metered portion of the biological sample is expelled through the capillary stop by air pressure produced within the air-bladder at step 1740 into the first conduit. Optionally at step 1750, the biological sample is moved forward within the first conduit to a portion of the first conduit (e.g., conduit 465 as described with respect to FIG. 4A) that is exposed to a sensor chip (e.g., sensor chip 1400 as described with respect to FIG. 14) by air pressure produced within the air-bladder such that the biological specimen can be amended with a compound or compounds (e.g., reagents such as enzymes, enzyme substrate, activators, stabilizers, buffers, enzyme-labeled antibody conjugate and the like) present initially as a dry coating on a portion of the sensor chip (i.e., one or more reagent regions). Additionally or alternatively, the fluid in the second conduit may be moved past the constriction into the first conduit and into contact with the biological specimen by air pressure produced by the first pump. The fluid may include a substrate that may be acted upon by the biological specimen and/or amended compounds to produce a chromatic substance. To facilitate the dissolution of the substrate, compound or compounds in the biological sample and/or promote efficient reaction, the biological sample may be oscillated by air pressure produced within the air-bladder. In one embodiment, an oscillation frequency of between about 0.2 Hz and about 5 Hz is used, most preferably about 0.7 Hz.

At step 1755, the biological sample is move forward within the first conduit to a portion of the first conduit forming an imaging chamber (e.g., imaging chamber 530 as described with respect to FIG. 5) that is exposed to the sensor chip (e.g., sensor chip 505 or 1400 as described with respect to FIGS. 5 and 14) by air pressure produced within the air-bladder such that analysis (e.g., optical analysis) of the biological specimen can be performed. Once the biological sample is move forward to the imaging chamber, the biological specimen is dispersed through-out the imaging chamber by capillary action. In various embodiments, the biological sample dispersed though-out the imaging chamber over the photosensitive surface such that one or more light emitters can transmit incident light of one or more wavelengths into the portion of the imaging chamber and the biological specimen. Upon the incident light striking the biological sample, photons that match an energy gap of cells, a target analyte, or a chromatic substance related to a presence of cells or the target analyte present in the biological specimen are absorbed. Other photons transmit through the imaging chamber and biological specimen unaffected. The photosensitive surface collect the photons of light transmitted through the imaging chamber and the biological sample, and convert the transmitted photons of light into current. At step 1760, the current is transmitted to the analyzer as an output signal via a conductive contact, and the analyzer compares the attenuation of the transmitted light with the incident light to obtain an absorption spectrum and converts the output signal to: (i) to a number count or percentage for each type of cell in the blood sample, or (ii) an analyte signal proportional to the light received from the imaging chamber and collected by the photosensitive surface.

At step 1765, the biological sample is move forward within the first conduit to an auxiliary conduit (e.g., auxiliary conduit 1525 as described with respect to FIG. 15) that is exposed to the sensor chip (e.g., sensor chip 1500/1510 as described with respect to FIG. 15) by air pressure produced within the air-bladder such that analysis (e.g., electrochemical analysis) of the biological specimen can be performed. Optionally at step 1770, the biological sample is moved forward such that the biological specimen can be amended with a compound or compounds (e.g., reagents such as an enzyme and enzyme substrate-labeled antibody conjugate) present initially as a dry coating on a portion of the sensor chip (i.e., one or more reagent regions). To facilitate the dissolution of the compound or compounds in the biological sample and/or promote efficient reaction, the biological sample may be oscillated over the one or more reagent regions by air pressure produced within the air-bladder. In one embodiment, an oscillation frequency of between about 0.2 Hz and about 5 Hz is used, most preferably about 0.7 Hz. At step 1775, the biological sample is moved forward within the auxiliary conduit to a position over a second sensor (e.g., an amperometric sensor) by air pressure produced within the air-bladder. Optionally at step 1780, to promote efficient reaction product formation sandwich formation on or near the surface of the second sensor comprising a biolayer, the biological sample may be oscillated over the second sensors by air pressure produced within the air-bladder. In one embodiment, an oscillation frequency of between about 0.2 Hz and about 5 Hz is used, most preferably about 0.7 Hz

At step 1785, the biological sample is displaced from the auxiliary conduit by further pressure applied to air-bladder, and the biological sample passes to a waste chamber (e.g., waste chamber 433 as described with respect to FIGS. 4A and 4G.). At optional step 1790, one or more air segments (meniscus) may be produced within the first conduit by any suitable means, including a passive means, an embodiment of which is described in detail in U.S. Pat. No. 7,682,833, which is incorporated herein by reference in its entirety, or an active means including a transient lowering of the pressure within the first conduit using the second pump whereby air is drawn into the first conduit through a flap or valve. The one or more air segments are extremely effective at clearing or rinsing the biological sample-contaminated fluid from the first conduit. For example, a leading and/or trailing edge of the one or more air segments may be passed a number of times over the first and second sensors to rinse and resuspend extraneous material that may have been deposited from the biological sample. Extraneous material includes any material other than specifically bound analyte or analyte/antibody-enzyme conjugate complex. However, in accordance with various embodiments, the clearing or rinsing step 1790 using the one or more air segments is not sufficiently protracted or vigorous so as to promote substantial dissociation of specifically bound analyte or analyte/antibody-enzyme conjugate complex from a biolayer.

Optionally at step 1795, the fluid in the second conduit is moved past the constriction into the auxiliary conduit and into contact with the second sensor by air pressure produced by the first pump. The fluid may include a substrate or signal agent and the enzyme remaining within the auxiliary conduit and immobilized on or near the second sensor either produces an electroactive species from an electro-inactive substrate or destroys an electroactive substrate. In some embodiments, the fluid may be applied to the second sensor to wash the biological sample from the second sensor. At step 1797, a change in current or potential generated by the production or destruction of the electroactive species at the second sensor and the change is transmitted as a function of time to the analyzer via a conductive contact, and the analyzer performs analysis of the change in current or potential to identify the presence and/or concentration of the target analyte in the biological specimen.

As should be understood, the previous steps could be split up into two or more processes for using two or more testing devices to perform an optical assay and an electrochemical assay in accordance with alternative embodiments of the invention. For example, the steps pertaining to the optical assay could be performed via an optical testing device and subsequently the steps pertaining to the electrochemical assay could be performed via an electrochemical testing device, or vice versa.

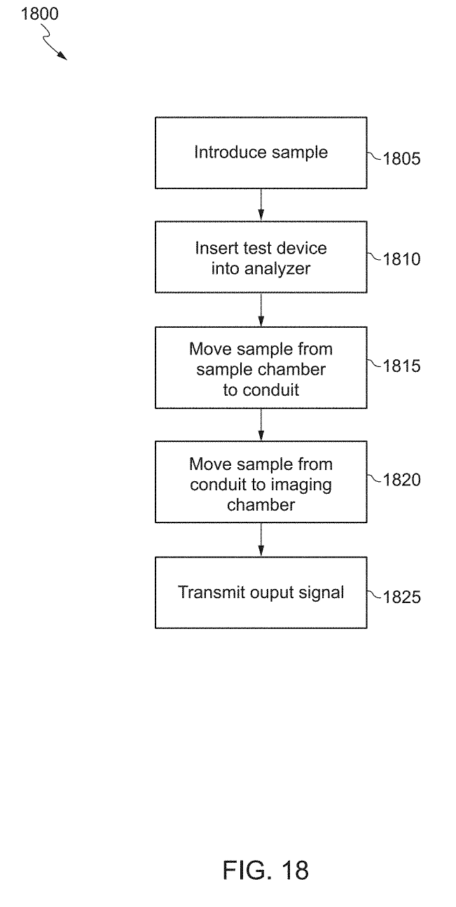

FIG. 18 illustrates a method 1800 (with reference to the testing device 400 as illustrated in FIGS. 4K-4R) of using a testing device to perform an optical assay in accordance with one embodiment of the invention. At step 1805, an unmetered biological sample may be introduced into a sample receiving chamber (e.g., the sample receiving chamber 420 described with respect to FIG. 4R) of a testing device, through a sample entry port (e.g., sample entry port 415 described with respect to FIGS. 4K and 4P). While the biological sample is within the sample chamber, the biological sample may be optionally passively amended (e.g., dissolution) with a compound or compounds (e.g., reagents such as dyes, enzymes, enzyme substrate, activators, stabilizers, binders, anticoagulants, buffers, enzyme-labeled antibody conjugate and the like) present initially as a dry coating on the inner surface of the sample chamber.

At step 1810, the testing device may be inserted into an analyzer (e.g., analyzer 105 described with respect to FIG. 1) in accordance with some aspects of the present invention. At step 1815, the biological sample moves passively from the sample chamber into a conduit (e.g., conduit 465 described with respect to FIG. 4R). For example, capillary action may facilitate the passive movement of the biological sample from the sample chamber into the conduit. In some embodiments, the surfaces of the fluidic paths and/or conduit may be treated to make them more or less hydrophilic and hydrophobic to further promote capillary action using established techniques known in the art. While the biological sample is within the conduit, the biological sample may be optionally amended passively (e.g., dissolution) with a compound or compounds (e.g., reagents such as dyes, enzymes, enzyme substrate, activators, stabilizers, binders, anticoagulants, buffers, enzyme-labeled antibody conjugate and the like) present initially as a dry coating on the inner surface of the conduit.

At step 1820, the biological sample moves passively through the conduit to a portion of the conduit forming an imaging chamber (e.g., imaging chamber 485, 530 as described with respect to FIGS. 4P and 5) that is exposed to the sensor chip (e.g., sensor chip 505 or 1400 as described with respect to FIGS. 5 and 14). For example, capillary action may facilitate the passive movement of the biological sample through the conduit and into the imaging chamber. While the biological sample is within the imaging chamber, the biological sample may be optionally amended passively (e.g., dissolution) with a compound or compounds (e.g., reagents such as dyes, enzymes, enzyme substrate, activators, stabilizers, binders, anticoagulants, buffers, enzyme-labeled antibody conjugate and the like) present initially as a dry coating on the inner surface of the imaging chamber.

Once the biological sample is move forward to the imaging chamber, the biological specimen is dispersed through-out the imaging chamber by capillary action. In various embodiments, the biological sample dispersed though-out the imaging chamber over the photosensitive surface such that one or more light emitters can transmit incident light of one or more wavelengths into the portion of the imaging chamber and the biological specimen. Upon the incident light striking the biological sample, photons that match an energy gap of cells, a target analyte, or a chromatic substance related to a presence of cells or the target analyte present in the biological specimen are absorbed. Other photons transmit through the imaging chamber and biological specimen unaffected. The photosensitive surface collect the photons of light transmitted through the imaging chamber and the biological sample, and convert the transmitted photons of light into current. At step 1825, the current is transmitted to the analyzer as an output signal via a conductive contact, and the analyzer compares the attenuation of the transmitted light with the incident light to obtain an absorption spectrum and converts the output signal to: (i) to a number count or percentage for each type of cell in the blood sample, or (ii) an analyte signal proportional to the light received from the imaging chamber and collected by the photosensitive surface.

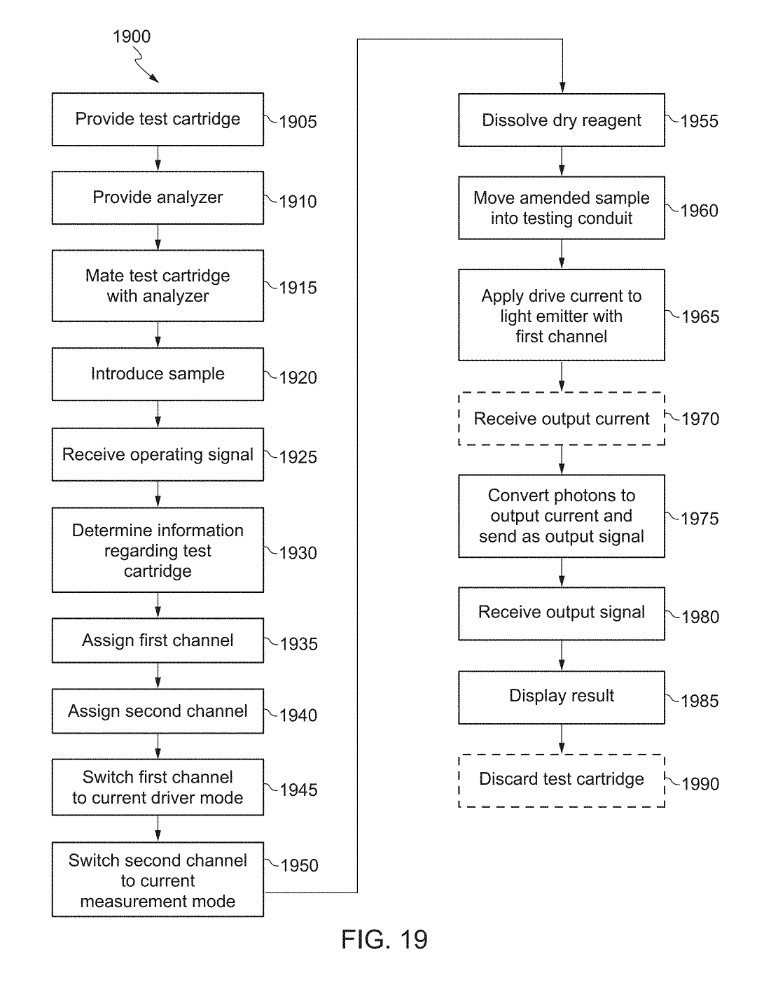

FIG. 19 illustrates a method 1900 of performing a differential blood cell count in a biological sample (e.g., whole blood) in accordance with various embodiments of the invention. At step 1905, a test cartridge is provided that comprises a sample entry port, a sample testing conduit fluidically connected to the sample entry port, optionally a pump, and an imager chip comprising an array of pixels. In some embodiments, the test cartridge further comprises a plurality of discrete connector contacts, and the imager chip is electrically connected to at least one of the plurality of discrete connector contacts. At step 1910, an analyzer is provided comprising a processor and display. In some embodiments, the analyzer further comprises a multi-terminal connector and optionally a pump actuator. At step 1915, the test cartridge is mated with the analyzer. The mating may comprise inserting the testing device into a port of the analyzer. Mating or inserting the test cartridge into the port of the analyzer places the multi-terminal connector in electrical contact with the plurality of discrete connector contacts. Mating or inserting the test cartridge into the port of the analyzer places may also place the pump actuator aligned with the pump in the test cartridge. At step 1920, a blood sample (e.g., whole blood) is introduced into the sample entry port before or after the mating the test cartridge with the analyzer.

At step 1925, an operating state signal is received that is indicative of a type of test cartridge inserted into the analyzer. In some embodiments, the operating state signal comprises a value of a measured resistance between contacts of the test cartridge and a shorting bar. For example, in order to impart cartridge identification functionality into a test cartridge, an additional mechanism or means may be included in the sensor chip arrangement for cartridge identification. In certain embodiments, a resistor can be implemented between contacts. The resistance of the resistor may be measured by a detector (e.g., processor) by applying a small voltage, e.g., 1 mV, between the contacts, subsequent to (e.g., immediately after) the cartridge being inserted into the analyzer. The value of the measured resistance can then be used for cartridge identification. For example, each cartridge type (e.g., i-STAT® cartridges EC8+, CG8+, EG7+, CHEM8+, etc.) may be associated with a certain resistance or resistance range such that a measured resistance of the cartridge may be used to identify the type of cartridge using a look-up table.

In alternative embodiments, the operating state signal comprises a value obtained from a barcode located on the test cartridge or a package of the test cartridge. For example, an imaging area of the test cartridge may be used to scan a barcode to obtain a value using the barcode reader 135 of the instrument 110, as described with respect to FIG. 1. The value of barcode can then be used for cartridge identification. For example, each cartridge type (e.g., i-STAT® cartridges EC8+, CG8+, EG7+, CHEM8+, etc.) may be associated with a certain value such that a scanned value of the cartridge may be used to identify the type of cartridge using a look-up table retained in the instrument.

At step 1930, information regarding sensors of the test cartridge are determined based on the identified type of cartridge. In certain embodiments, determining the information comprises: identifying, based on a value of the operating state signal, the type of test cartridge using a look-up table, and obtaining, based on the type of test cartridge, the information regarding the sensors from a database, where the database has information for each type of test cartridge. In various embodiments, the information indicates the type of sensors of the test cartridge (e.g., one or more optical sensors, one or more reference electrode, one or more electrochemical sensors, etc) and the position of conductive contacts connected to the sensors of the test cartridge In addition or alternative to obtaining information regarding the type of sensors and the position of conductive contacts from the database via the identified type of testing cartridge, the type of sensors and position of the conductive contacts may be identified using information obtained regarding the connector pins in contact with the various conductive contacts of the testing cartridge. For example, the analyzer connector may be a linear array of connector pins, e.g., pins one to twenty. The type of sensors and position of the conductive contacts may be identified via the position of each pin relative to the contacts. For example, a light emitter may be connected via a contact to a pin “x” (e.g., pin 11) and a light detector of the optical sensor may be connected via another contact to a pin “y” (e.g., 12), and thus since both pins 11 and 12 are being used, the type of sensor (optical) and components (e.g., light emitter and light detector) connected to the contacts can be identified via the database. Consequently, as described herein, the analyzer may then assign channels of the universal circuitry to the appropriate pins for the types of sensors determined to be in the identified testing cartridge. As should be understood, once a test cycle is run and the testing cartridge is removed from the instrument or analyzer, the channels of the universal circuitry can be reassigned to the same or different connector pins when a new testing cartridge is inserted into the analyzer.

At step 1935, a first channel is assigned to the light emitter via: (i) the first contact and a corresponding first pin, and optionally, (ii) the second contact and a corresponding second pin. At step 1940, a second channel is assigned to the light detector via the third contact and a corresponding third pin. At step 1945, the circuitry of the first channel is switched to a current driver mode. In some embodiments, the switching the circuitry of the first channel comprises modifying switching elements of the circuitry such that the first channel is configured to apply the drive current via the first contact and the corresponding first pin to the light emitter. At step 1950, the circuitry of the second channel is switched to a current measurement mode. In some embodiments, the switching the circuitry of the second channel comprises modifying switching elements of the circuitry such that the second channel is configured to convert output current received from the light detector to a measurable voltage proportional to an amount light detected by the light detector.

At step 1955, a dry reagent is dissolved into the blood sample to generate an amended blood sample. In some embodiments, dissolving the dry reagent may include driving the pump actuator to actuate the pump on the test cartridge and move the blood sample into contact with the dry reagent (e.g., cause the blood sample to oscillate over the dry reagent), which ultimately dissolves the dry reagent in the blood sample. In other embodiments, dissolving the dry reagent may include the blood sample moving passively into contact with the dry reagent, which ultimately dissolves the dry reagent in the blood sample. At step 1960, the amended blood sample is moved into a sample testing conduit. The sample testing conduit may comprise a first wall formed from at least a portion of an imager chip, a second wall formed from a transparent material layer, and a plurality of spacer elements having an average spacer height and disposed between the first wall and the second wall. In certain embodiments, the average spacer height defines an average chamber height of a chamber between the portion of the imager chip and the transparent material layer. In some embodiments, moving the blood sample into the sample testing conduit includes driving the pump actuator to actuate the pump on the test cartridge and move the blood sample from a sample receiving chamber into the sample testing conduit. In other embodiments, moving the blood sample into the sample testing conduit includes the blood sample moving passively from a sample receiving chamber into the sample testing conduit.

At step 1965, a drive current is applied to the light emitter using the first channel. The applying the drive current to the light emitter causes the light emitter to generate output current and light comprising a predetermined wavelength that is projected through the chamber and the amended blood sample. Optionally at step 1970, the output current generated by the light emitter is received at the first channel from the second contact and the corresponding second pin, and the output current is applied to a feedback resistor to establish a constant current for the drive current.

At step 1975, the light detector converts the photons of light received from the light emitter to an output current and sends the output current to the third contact as an output signal. In some embodiments, the output signal is at least one of absorbance and fluorescence and is recorded at the array of pixels based on the light received from the light emitter. At step 1980, the output signal from the light detector is received at the second channel via the third contact and the corresponding third pin. The output signal may be converted, using the second channel, to a number count or percentage for each type of cell in the blood sample. At step 1885, the number count or percentage for each type of cell in the blood sample may be displayed on the display. Optionally at step 1990, the test cartridge is unmated from the analyzer and the test cartridge is discarded in the trash.

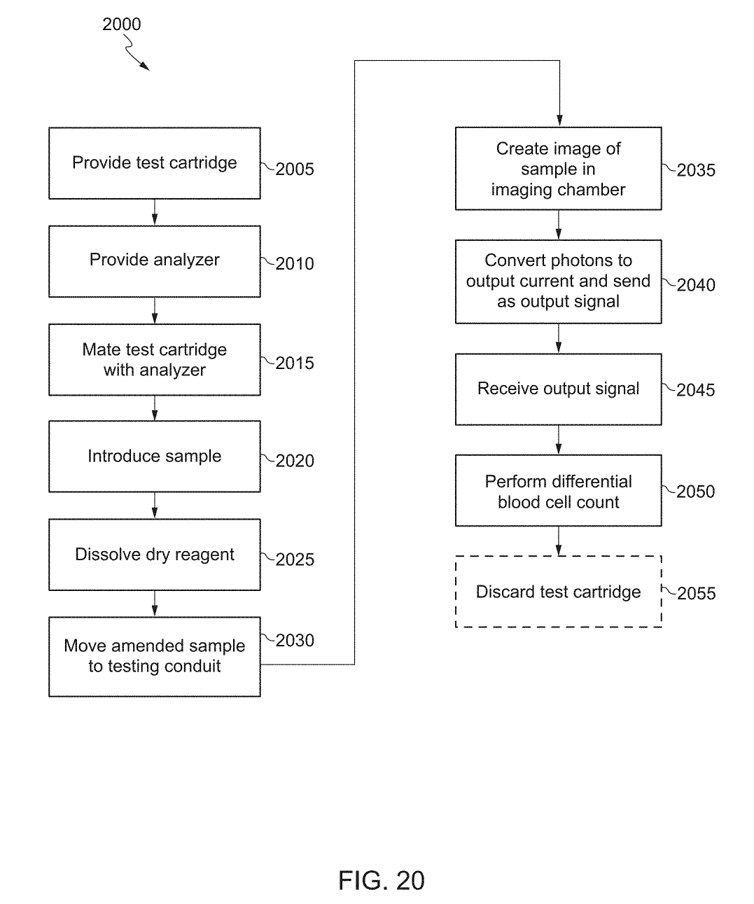

FIG. 20 illustrates a method 2000 of performing a differential blood cell count in a biological sample (e.g., whole blood) in accordance with various embodiments of the invention. Medical diagnostics often include analyses of a whole blood sample from a patient. One of the more popular diagnostics is a complete blood count (referred to as a “CBC”), which is a suite of tests that may include, in addition to the enumeration of the cellular components, red blood cell metrics, reticulocyte counts, and a leukocyte differential count (“LDC”; sometimes referred to as a “white blood cell differential”), which is the identification and enumeration of the types of white blood cells (WBCs) present in the blood sample. At step 2005, a test cartridge is provided that comprises a sample entry port, a sample testing conduit fluidically connected to the sample entry port, optionally a pump, and an imager chip comprising an array of pixels. In some embodiments, the test cartridge further comprises a plurality of discrete connector contacts, and the imager chip is electrically connected to at least one of the plurality of discrete connector contacts. At step 2010, an analyzer is provided comprising a processor and display. In some embodiments, the analyzer further comprises a multi-terminal connector and optionally a pump actuator. At step 2015, the test cartridge is mated with the analyzer. The mating may comprise inserting the testing device into a port of the analyzer. Mating or inserting the test cartridge into the port of the analyzer places the multi-terminal connector in electrical contact with the plurality of discrete connector contacts. Mating or inserting the test cartridge into the port of the analyzer places may also place the pump actuator aligned with the pump in the test cartridge. At step 2020, a blood sample (e.g., whole blood) is introduced into the sample entry port before or after the mating the test cartridge with the analyzer.

At step 2025, a dry reagent is dissolved into the blood sample to generate an amended blood sample. In some embodiments, dissolving the dry reagent may include driving the pump actuator to actuate the pump on the test cartridge and move the blood sample into contact with the dry reagent (e.g., cause the blood sample to oscillate over the dry reagent), which ultimately dissolves the dry reagent in the blood sample. In other embodiments, dissolving the dry reagent may include the blood sample moving passively into contact with the dry reagent, which ultimately dissolves the dry reagent in the blood sample. The reagent may be added to the sample to facilitate distinguishing one constituent from another within the sample. For example, the reagent may be a dye or colorant such as Acridine Orange (also referred to as “Basic Orange 15” or “ACO”) and Astrazon Orange (also referred to as “AO” or Basic Orange 21), which emit light at particular wavelengths when mixed with whole blood and subjected to an excitation wavelength from the light emitter. The light emitter may be operable to produce light at wavelengths associated with one or more of red, green, and blue light. The red light is typically produced in the range of about 600-700 nm, with red light at about 660 nm preferred. The green light is typically produced in the range of about 515-570 nm, with green light at about 540 nm preferred. The blue light is typically in the range of about 405-425 nm, with blue light at about 413 nm preferred. Light transmitted through the sample, or fluoresced from the sample, is captured using the imager chip, and a signal representative of the captured light is sent to the analyzer, where it is processed into an image. The image is produced in a manner that permits the light transmittance or fluorescence intensity captured within the image to be determined on a per unit basis; e.g., “per unit basis” being an incremental unit of which the image of the sample can be dissected, such as a pixel.

At step 2030, the amended blood sample is moved into a sample testing conduit. In certain embodiments, the sample is quiescently residing within the chamber. The sample testing conduit may comprise a first wall formed from at least a portion of an imager chip, a second wall formed from a transparent material layer, and a plurality of spacer elements having an average spacer height and disposed between the first wall and the second wall. In certain embodiments, the average spacer height defines an average chamber height of a chamber between the portion of the imager chip and the transparent material layer. In some embodiments, moving the blood sample into the sample testing conduit includes driving the pump actuator to actuate the pump on the test cartridge and move the blood sample from a sample receiving chamber into the sample testing conduit. In other embodiments, moving the blood sample into the sample testing conduit includes the blood sample moving passively from a sample receiving chamber into the sample testing conduit.

At step 2035, at least one image of the sample residing within the chamber is created using the imager chip. In some embodiments, the imager chip comprises a CCD type image sensor that converts light passing through (or from) the sample into an electronic data format image. In other embodiments, the imager chip comprises a CMOS type image sensor that converts light passing through (or from) the sample into an electronic data format image. The signals from the imager chip provide information for each pixel of the image, which information includes, or can be derived to include, intensity, wavelength, and optical density. Intensity values may be assigned an arbitrary scale of, for example, 0 units to 4095 units (“IVUs”). Optical density (“OD”) is a measure of the amount of light absorbed relative to the amount of light transmitted through a medium; e.g., the higher the “OD” value, the greater the amount of light absorbed during transmission. OD may be quantitatively described in optical density units (“OD”) or fractions thereof; e.g., a MilliOD is a 1/1000^{th} of an OD. One “OD” unit decreases light intensity by 90%. “OD” or “MilliOD” as a quantitative value can be used for images acquired or derived by transmission light, for example, the transmission blue light.

In some embodiments, the information from the imager chip is separated into multiple channels, for example, three channels, which provides particular utility for determining a four part LDC. However, the present invention is not limited to a three channel embodiment. A first of the three channels may be directed toward information relating to light emitted from the sample at a first wavelength (e.g., 540 nm, which appears green). A second channel may be directed toward information relating to light emitted from the sample at a second wavelength (e.g., 660 nm, which appears red). A third channel may be directed toward information relating to light passing through the sample at a third wavelength (e.g., 413 nm, which is used to determine blue optical density—“OD”). These wavelength values and the number of channels have particular utility when an LDC is being performed on a whole blood sample. However, the present invention is not limited to these particular wavelengths or number of channels. Additional channels can be implemented to gather information at different wavelengths and/or transmission values. That information, in turn, can be used to evaluate additional constituents within the sample and/or to increase the accuracy of the analysis. For example, in applications where it is desirable to further differentiate basophils within the sample, a fourth and a fifth channel can be added. The fourth channel can be directed toward information relating to light passing through the sample at a fourth wavelength (e.g., 540 nm), which is used to determine green OD, and the fifth channel can be directed toward information relating to light passing through the sample at a fifth wavelength (e.g., 660 nm), which is used to determine red OD. These OD values, in turn, can be used to identify basophils.

At step 2040, the imager chip converts the photons of light received from the light emitter to an output current and sends the output current to the analyzer. At step 2045, the output signal from the imager chip is received at the analyzer. For example, the analyzer is in communication with the test cartridge, light emitter, and imager chip, and may be adapted (e.g., programmed) to send and receive signals from one or more of the cartridge, light emitter, and imager chip. For example, the analyzer is adapted to: (i) send signals to the light emitter to produce light at defined wavelengths (or alternatively at multiple wavelengths); and (ii) send and receive signals from the imager chip to capture light for defined periods of time. The analyzer is further adapted to process the signals (e.g., the output signal) received from the imager chip according to one or more predetermined algorithms. The specifics of a particular algorithm will depend upon the analysis at hand. As indicated above, the present invention has particular utility when applied to perform an LDC on a whole blood sample, and to illustrate that utility the invention is described herein as performing an LDC. However, the present invention is not limited to this particular analysis.

At step 2050, a differential blood cell count is performed using the output signal received from the imager chip. In some embodiments, a differential blood cell count includes: (i) identifying the cells, for example white blood cells, within the sample residing within the chamber; (ii) quantitatively analyzing at least some of the identified cells within the image relative to one or more predetermined quantitatively determinable features; and (iii) identifying at least one type of cell from the identified cells using the quantitatively determinable features. For example, to perform the differential blood cell count such as a LDC, the algorithm utilizes a set of identifying features, each of which features is distinguishable from the other features and each of which is quantitatively determinable from an image of the sample. Each WBC can be characterized by the presence or absence of certain identifying features, and/or by quantitative information associated with certain features. For purposes of providing an enabling disclosure, the present invention is described herein in terms of an exemplary set of identifying features that can be used to selectively identify and distinguish WBCs. This set is not inclusive of all possible features, and therefore the present invention is not limited to this particular set.

For a WBC analysis, if for example acridine orange is used, an exemplary set of identifying features includes those entitled: Cell, Nucleus, number of Lobes, Cell Area, Nucleus Area Ratio of Large Granules, Ratio of Nucleus, Red-Green Ratio, Nucleus Shape, Cell Shape, Nucleus Brightness, Cytoplasm Brightness, Average Cell Absorption at a Given Wavelength, Nucleus Texture, Cytoplasm Texture, Cell Absorption Texture at a Given Wavelength, Nucleus Hollowness, and Cytoplasm Hollowness; each of which is described in US. Patent Publication No. 20120034647, which is incorporated herein by reference. In some instances, certain features directly provide information about a particular cell (e.g., Nucleus Shape). In other instances, a feature (e.g., Cell Area) can be used to indirectly provide information about a particular cell (e.g., ratio of Nucleus Area to Cell Area—referred to above as “Ratio of Nucleus”, etc.). The identifying features are based on quantifiable characteristics such as light intensity, light color. OD, area, and relative position (e.g., shape). As indicated above, the colors may be created by one or more fluorescent colorants admixed with the sample, which upon excitation, produce fluorescent light emission at particular wavelengths associated with particular colors. As should be understood, this principal also applies to non-fluorescent dye detection based on absorbance of a particular wavelength associated with particular colors.

An example of an acceptable colorant that can be used when performing an LDC on a whole blood sample is Acridine Orange (“ACO”). ACO is a fluorescent dye that, when mixed with a whole blood sample, selectively stains constituents within the sample; e.g., white blood cells, platelets, reticulocytes, and nucleated red blood cells. With respect to WBCs, the ACO permeates through the respective WBC and stains its DNA and RNA. The color(s) emitted by the dye within the WBC arc a function of a number of factors, including: the quantity of RNA and DNA within the dye, the concentration of the dye in the constituent, and the pH of the constituent. The present invention is not limited to using ACO, and other dyes (e.g., Astrazon Orange) may be used in place of ACO or in combination with ACO. Using ACO and white blood cells as an example, if the sample is subjected to an excitation light at or about a wavelength of 470 nm, the ACO bound to materials (e.g., DNA) within the nucleus of a white blood cell will emit light at about 540 nm (which appears green), and the ACO bound to materials (e.g., RNA) within the cytoplasm of a white blood cell will emit light at about 660 nm (which appears red).

As indicated above, OD values within the sample are a function of absorptivity of light at predetermined wavelengths by materials that naturally occur within the cell (e.g., hemoglobin), and/or may be a function of colorant absorbed (or not absorbed) by constituents within the sample. The identification of particular groups of pixels at one or more defined wavelengths can be performed using a variety of different techniques. For example, segmentation techniques can be used to produce a masked image depicting only those pixels within the image that meet the criteria (e.g., intensity and color). For those analyses that derive information only from the green light portions (e.g., the nuclei) or the red light portions (e.g., cytoplasm) of the image, or both, the sample image can be masked to produce a partial image depicting only those pixels showing green, or red, or both, and may also be distinguished by a predetermined intensity threshold. The present invention is not limited to any particular segmentation technique, and a specific technique can be chosen in view of the application at hand. For example, a hard segmentation technique can be used wherein a pixel is assigned as either belonging to an object or not. “Hard” segmentation techniques can be implemented using thresholding, region grow, or watershed type routines. Alternatively, soft segmentation techniques can be utilized; e.g., a “fuzzy” segmentation, where each pixel is assigned a value in the range of 0 to 1, which value describes the likelihood that the particular pixel belongs to the object. The description of each of the identifying features below will provide clear examples of how quantitative data such as that associated with wavelength and intensity can provide a basis for distinguishing one WBC from another. The present invention is also not limited to using a segmentation technique, and can use other techniques that select (i.e., “pick”) pixels or otherwise distinguish pixels having particular attributes.

As indicated above, an LDC is an analysis wherein the different types of WBCs are identified and enumerated. The results can be expressed in terms of the relative percentages of the identified WBC types. Consequently, at step 2050, the output signal output signal may be converted, using the analyzer, to a number count or percentage for each type of cell in the blood sample. At step 2050, the number count or percentage for each type of cell in the blood sample may be displayed on the display. Optionally at step 2055, the test cartridge is unmated from the analyzer and the test cartridge is discarded in the trash.

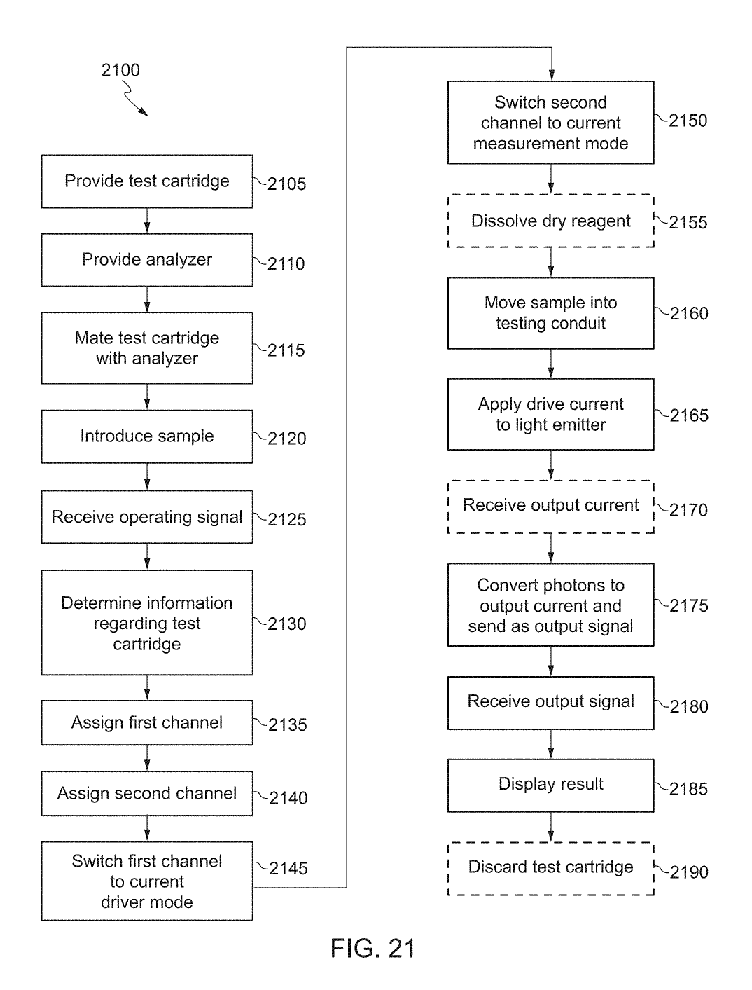

FIG. 21 illustrates a method 2100 of imaging assay beads in a biological sample (e.g., plasma) in accordance with various embodiments of the invention. At step 2105, a test cartridge is provided that comprises a sample entry port, a sample testing conduit fluidically connected to the sample entry port, optionally a pump, and an imager chip comprising an array of pixels. In some embodiments, the test cartridge further comprises a plurality of discrete connector contacts, and the imager chip is electrically connected to at least one of the plurality of discrete connector contacts. At step 2110, an analyzer is provided comprising a processor and display. In some embodiments, the analyzer further comprises a multi-terminal connector and optionally a pump actuator. At step 2115, the test cartridge is mated with the analyzer. The mating may comprise inserting the testing device into a port of the analyzer. Mating or inserting the test cartridge into the port of the analyzer places the multi-terminal connector in electrical contact with the plurality of discrete connector contacts. Mating or inserting the test cartridge into the port of the analyzer places may also place the pump actuator aligned with the pump in the test cartridge. At step 2020, a blood sample is introduced into the sample entry port before or after the mating the test cartridge with the analyzer.

At step 2125, an operating state signal is received that is indicative of a type of test cartridge inserted into the analyzer. In some embodiments, the operating state signal comprises a value of a measured resistance between contacts of the test cartridge and a shorting bar. For example, in order to impart cartridge identification functionality into a test cartridge, an additional mechanism or means may be included in the sensor chip arrangement for cartridge identification. In certain embodiments, a resistor can be implemented between contacts. The resistance of the resistor may be measured by a detector (e.g., processor) by applying a small voltage, e.g., 1 mV, between the contacts, subsequent to (e.g., immediately after) the cartridge being inserted into the analyzer. The value of the measured resistance can then be used for cartridge identification. For example, each cartridge type (e.g., i-STAT® cartridges EC8+, CG8+, EG7+, CHEM8+, etc.) may be associated with a certain resistance or resistance range such that a measured resistance of the cartridge may be used to identify the type of cartridge using a look-up table.

In alternative embodiments, the operating state signal comprises a value obtained from a barcode located on the test cartridge or a package of the test cartridge. For example, an imaging area of the test cartridge may be used to scan a barcode to obtain a value using the barcode reader 135 of the instrument 110, as described with respect to FIG. 1. The value of barcode can then be used for cartridge identification. For example, each cartridge type (e.g., i-STAT® cartridges EC8+, CG8+, EG7+, CHEM8+, etc.) may be associated with a certain value such that a scanned value of the cartridge may be used to identify the type of cartridge using a look-up table retained in the instrument.

At step 2130, information regarding sensors of the test cartridge are determined based on the identified type of cartridge. In certain embodiments, determining the information comprises: identifying, based on a value of the operating state signal, the type of test cartridge using a look-up table, and obtaining, based on the type of test cartridge, the information regarding the sensors from a database, where the database has information for each type of test cartridge. In various embodiments, the information indicates the type of sensors of the test cartridge (e.g., one or more optical sensors, one or more reference electrode, one or more electrochemical sensors, etc.) and the position of conductive contacts connected to the sensors of the test cartridge In addition or alternative to obtaining information regarding the type of sensors and the position of conductive contacts from the database via the identified type of testing cartridge, the type of sensors and position of the conductive contacts may be identified using information obtained regarding the connector pins in contact with the various conductive contacts of the testing cartridge. For example, the analyzer connector may be a linear array of connector pins, e.g., pins one to twenty. The type of sensors and position of the conductive contacts may be identified via the position of each pin relative to the contacts. For example, a light emitter may be connected via a contact to a pin “x” (e.g., pin 11) and a light detector of the optical sensor may be connected via another contact to a pin “y” (e.g., 12), and thus since both pins 11 and 12 are being used, the type of sensor (optical) and components (e.g., light emitter and light detector) connected to the contacts can be identified via the database. Consequently, as described herein, the analyzer may then assign channels of the universal circuitry to the appropriate pins for the types of sensors determined to be in the identified testing cartridge. As should be understood, once a test cycle is run and the testing cartridge is removed from the instrument or analyzer, the channels of the universal circuitry can be reassigned to the same or different connector pins when a new testing cartridge is inserted into the analyzer.

At step 2135, a first channel is assigned to the light emitter via: (i) the first contact and a corresponding first pin, and optionally, (ii) the second contact and a corresponding second pin. At step 2140, a second channel is assigned to the light detector via the third contact and a corresponding third pin. At step 2145, the circuitry of the first channel is switched to a current driver mode. In some embodiments, the switching the circuitry of the first channel comprises modifying switching elements of the circuitry such that the first channel is configured to apply the drive current via the first contact and the corresponding first pin to the light emitter. At step 2150, the circuitry of the second channel is switched to a current measurement mode. In some embodiments, the switching the circuitry of the second channel comprises modifying switching elements of the circuitry such that the second channel is configured to convert output current received from the light detector to a measurable voltage proportional to an amount light detected by the light detector.

Optionally, at step 2155, a dry reagent is dissolved into the blood sample to generate an amended blood sample. In some embodiments, dissolving the dry reagent may include driving the pump actuator to actuate the pump on the test cartridge and move the blood sample into contact with the dry reagent (e.g., cause the blood sample to oscillate over the dry reagent), which ultimately dissolves the dry reagent in the blood sample. In other embodiments, dissolving the dry reagent may include the blood sample moving passively into contact with the dry reagent, which ultimately dissolves the dry reagent in the blood sample. At step 2160, the amended blood sample is moved into a sample testing conduit. The sample testing conduit may comprise a first wall formed from at least a portion of an imager chip, a second wall formed from a transparent material layer, and a plurality of wells having an average well height and disposed between the first wall and the second wall. In some embodiments, each of the plurality of wells is aligned vertically with one or more pixels of the imager chip, and at least a portion of the plurality of wells comprise at least one assay bead. In some embodiments, moving the blood sample into the sample testing conduit includes driving the pump actuator to actuate the pump on the test cartridge and move the blood sample from a sample receiving chamber into the sample testing conduit. In other embodiments, moving the blood sample into the sample testing conduit includes the blood sample moving passively from a sample receiving chamber into the sample testing conduit.

At step 2165, a drive current is applied to the light emitter using the first channel. The applying the drive current to the light emitter causes the light emitter to generate output current and light comprising a predetermined wavelength that is projected through the sample testing conduit and the amended blood sample. Optionally, at step 2170, the output current generated by the light emitter is received at the first channel from the second contact and the corresponding second pin, and the output current is applied to a feedback resistor to establish a constant current for the drive current.

At step 2175, the light detector converts the photons of light received from the light emitter to an output current and sends the output current to the third contact as an output signal. In some embodiments, the output signal is at least one of absorbance and fluorescence and is recorded at the array of pixels based on the light received from the light emitter. At step 2180, the output signal from the light detector is received at the second channel via the third contact and the corresponding third pin. The output signal may be converted, using the second channel, to a value indicative of a reaction of the biological sample with the at least one assay bead in each of the plurality of wells. At step 2185, value indicative of a reaction of the biological sample may be displayed on the display. Optionally at step 2190, the test cartridge is unmated from the analyzer and the test cartridge is discarded in the trash.

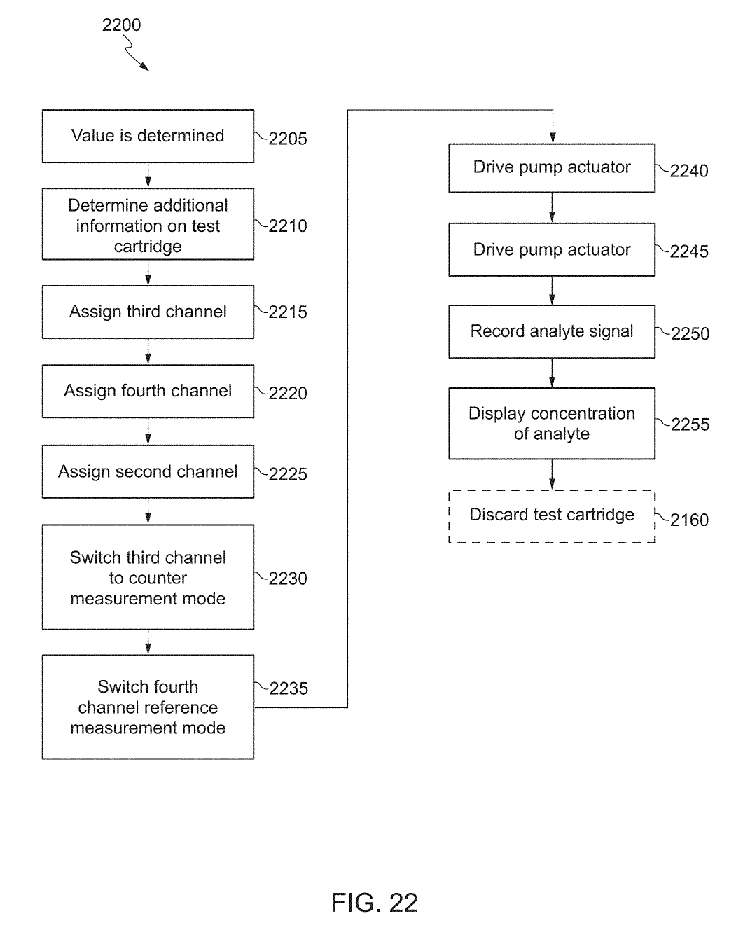

FIG. 22 illustrates a method 2200 of performing an optical assay and electrochemical assay using a same testing device. At step 2205, a qualitative, semi-quantitative, or quantitative value is determined based on a measurable voltage that is proportional to cell types or an amount of target analyte in the biological specimen in accordance with steps 1905-1985 of method 1900 or 2105-2185 of method 2100. At step 2210, additional/alternative information regarding sensors of the test cartridge is determined based on the type of the test cartridge and/or the pins being used. In various embodiments, the information indicates that a fourth contact is connected to a counter electrode, a fifth contact is connected to a reference electrode, and the third contact or a sixth contact is connected to a working electrode (e.g., an amperometric electrode).

At step 2215, a third channel is assigned to the counter electrode via the fourth contact and a corresponding fourth pin. At step 2220, a fourth channel is assigned to the reference electrode via the fifth contact and a corresponding fifth pin. At step 2225, the second channel is assigned to the working electrode via the third contact and the corresponding third pin or the sixth contact and a corresponding sixth pin. At step 2230, the circuitry of the third channel is switched to a counter measurement mode. In some embodiments, the switching the circuitry of the third channel comprises modifying switching elements of the circuitry such that the third channel is configured to apply a potential that is optionally not measured and is adjusted so as to balance the reaction occurring at the working electrode. This configuration allows the potential of the working electrode to be measured against a known electrode (i.e., the counter electrode) without compromising the stability of the reference electrode by passing current over the reference electrode. At step 2235, the circuitry of the fourth channel is switched to a reference measurement mode. In some embodiments, the switching the circuitry of the fourth channel comprises modifying switching elements of the circuitry such that the fourth channel is configured to apply a stable potential to the reference electrode, which may be used as a reference for measurements made by the working electrode.

At step 2240, the pump actuator is driven to actuate the pump on the test cartridge to split the blood sample into a first portion and a second portion. In some embodiments, the first portion of the blood sample is moved into the sample testing conduit. At step 2245, the pump actuator is driven to actuate the pump on the test cartridge to move the second portion of the blood sample into an auxiliary conduit comprising an electrochemical sensor for detecting an analyte in the blood sample. At step 2250, an analyte signal from the electrochemical sensor is recorded based on performance of an electrochemical analytical test in the auxiliary conduit, and a qualitative, semi-quantitative, or quantitative value is determined proportional to an amount of the analyte in the blood sample based on the analyte signal. In various embodiments, the performing the electrochemical analytical test comprises: (i) applying a potential to the counter electrode using the third channel; (ii) applying a potential to the reference electrode using the fourth channel; (iii) applying a potential to the working electrode using the second channel; (iv) measuring a current change across the biological specimen, using the second channel, that is proportional to a concentration of target analyte within the biological specimen; and (v) determining the concentration of target analyte within the biological specimen based on the current change across the biological specimen. In various embodiments, the counter electrode and the reference electrode are used in conjunction with the working electrode to measure the current change across the biological specimen. At step 2255, the concentration of target analyte within the biological specimen may be displayed on the display. Optionally at step 2260, the test cartridge is unmated from the analyzer and the test cartridge is discarded in the trash.

While the invention has been described in detail, modifications within the spirit and scope of the invention will be readily apparent to the skilled artisan. It should be understood that aspects of the invention and portions of various embodiments and various features recited above and/or in the appended claims may be combined or interchanged either in whole or in part. In the foregoing descriptions of the various embodiments, those embodiments which refer to another embodiment may be appropriately combined with other embodiments as will be appreciated by the skilled artisan. Furthermore, the skilled artisan will appreciate that the foregoing description is by way of example only, and is not intended to limit the invention.

We claim:

1. A method for performing a differential blood cell count comprising:
providing a test cartridge comprising a sample entry port, a sample testing conduit fluidically connected to the sample entry port, and an imager chip comprising an array of pixels;
providing an analyzer comprising a processor and display;
mating the test cartridge with the analyzer;
introducing a blood sample into the sample entry port before or after the mating the test cartridge with the analyzer;
dissolving a dry reagent into the blood sample to generate an amended blood sample;
moving the amended blood sample into the sample testing conduit, wherein the sample testing conduit comprises a first wall formed from at least a portion of an imager chip, a second wall formed from a transparent material layer, and a plurality of spacer elements having an average spacer height and disposed between the first wall and the second wall, and wherein the average spacer height defines an average chamber height of a chamber between the portion of the imager chip and the transparent material layer;
driving a light emitter to project light through the chamber and the amended blood sample;
recording an output signal of at least one of absorbance and fluorescence at the array of pixels based on the light received from the chamber and the amended blood sample;
converting the output signal using the processor to a number count or percentage for each type of cell in the blood sample; and
displaying the number count or percentage for each type of cell in the blood sample on the display.

2. The method of claim 1, further comprising:
unmating the test cartridge from the analyzer; and
disposing of the test cartridge.

3. The method of claim 1, wherein the dry reagent comprises acridine orange or an anticoagulant.

4. The method of claim 1, wherein the dry reagent binds to nucleic DNA in the blood cells.

5. The method of claim 1, wherein the mating comprises inserting the test cartridge into a port of the analyzer.

6. The method of claim 5, wherein the analyzer further comprises a multi-terminal connector, the test cartridge further comprises a plurality of discrete connector contacts, the imager chip is electrically connected to at least one of the plurality of discrete connector contacts, and the inserting the test cartridge into the port of the analyzer places the multi-terminal connector in electrical contact with the plurality of discrete connector contacts.

7. The method of claim 6, wherein the processor is electrically connected to the light emitter, the processor is electrically connected to the imager chip via the at least one of the plurality of discrete connector contacts and the multi-terminal connector, the light emitter is driven via the processor to project the light, and the imager chip is controlled via the processor to record the output signal.

8. The method of claim 5, the analyzer further comprises a pump actuator, the test cartridge further comprises a pump, the inserting the test cartridge into the port of the analyzer places the pump actuator aligned with the pump, and moving the amended blood sample into the sample testing conduit comprises driving the pump actuator to actuate the pump and displace the amended blood sample into the sample testing conduit.

9. A system comprising:
one or more processors; and
memory coupled to the one or more processors, the memory encoded with a set of instructions configured to perform a process comprising:
receiving an operating state signal from a test cartridge indicative of a type of cartridge inserted into an analyzer;
determining that the type of cartridge is the test cartridge having a contact connected to an imager chip configured to image blood cells in a blood sample;
driving a pump actuator to actuate a pump on the test cartridge and move the blood sample from a sample receiving chamber into a sample testing conduit, wherein the sample testing conduit comprises a first wall formed from at least a portion of the imager chip, a second wall formed from a transparent material layer, and a plurality of spacer elements disposed between the first wall and the second wall;
driving a light emitter to project light through the sample testing conduit and the blood sample;
recording an output signal of at least one of absorbance and fluorescence at an array of pixels of the imager chip based on the light received from the sample testing conduit and the blood sample; and
converting the output signal to a number count or percentage for each type of cell in the blood sample.

10. The system of claim 9, wherein the method further comprises driving the pump actuator to actuate the pump on the test cartridge and move the blood sample into contact with a dry reagent.

11. The system of claim 10, wherein the dry reagent comprises acridine orange or an anticoagulant.

12. The system of claim 10, wherein the dry reagent binds to nucleic DNA in the blood cells.

13. The system of claim 10, wherein the dry regent is disposed in the sample receiving chamber.

14. The system of claim 10, wherein the dry regent is disposed in the sample testing conduit.

15. The system of claim 9, further comprising displaying the number count or percentage for each type of cell in the blood sample on a display.

16. The system of claim 9, wherein the light emitter projects the light through the transparent material layer, the sample testing conduit and the blood sample.

17. The system of claim 16, wherein the light emitter is disposed in the test cartridge, and determining that the type of cartridge is the test cartridge having the contact connected to the imager chip and an another contact connected to the light emitter.

18. The system of claim 9, wherein the plurality of spacer elements have a predetermined average spacer height that defines a predetermined average chamber height of a chamber between the portion of the imager chip and the transparent material layer.

19. The system of claim 9, wherein at least one of the second wall and the plurality of spacer elements is deformable such that the second wall and the plurality of spacer elements are drawn toward each other by capillary force from the blood sample being moved into the sample testing conduit.

20. A non-transitory machine readable storage medium storing instructions that, when executed by one or more processors of a computing system, cause the computing system to perform operations comprising:
driving a pump actuator to actuate a pump on a test cartridge and move a blood sample from a sample receiving chamber into a sample testing conduit, wherein the sample testing conduit comprises a first wall formed from at least a portion of an imager chip, a second wall formed from a transparent material layer, and a plurality of spacer elements disposed between the first wall and the second wall;
driving a light emitter to project light through the sample testing conduit and the blood sample;
recording an output signal of at least one of absorbance and fluorescence at an array of pixels of the imager chip based on the light received from the sample testing conduit and the blood sample; and
converting the output signal to a number count or percentage for each type of cell in the blood sample.

21. The non-transitory machine readable storage medium of claim 20, wherein the operations further comprise driving the pump actuator to actuate the pump on the test cartridge to split the blood sample into a first portion and a second portion, wherein the first portion of the blood sample is moved into the sample testing conduit.

22. The non-transitory machine readable storage medium of claim 21, wherein the operations further comprise driving the pump actuator to actuate the pump on the test cartridge to move the second portion of the blood sample into an auxiliary conduit comprising an electrochemical sensor for detecting an analyte in the blood sample.

23. The non-transitory machine readable storage medium of claim 22, wherein the operations further comprise recording an analyte signal from the electrochemical sensoheightr based on performance of an electrochemical analytical test in the auxiliary conduit, and determining a qualitative, semi-quantitative, or quantitative value proportional to an amount of the analyte in the blood sample based on the analyte signal.

24. The non-transitory machine readable storage medium of claim 23, wherein the performing the electrochemical analytical test comprises applying a potential to the electrochemical sensor with respect to a reference electrode, and measuring a current change across the blood sample that is proportional to the amount of the analyte within the blood sample, and wherein the analyte signal is recorded as indicative of the measured current change across the blood sample.

25. The non-transitory machine readable storage medium of claim 24, wherein the operations further comprise receiving an operating state signal from the test cartridge indicative of a type of cartridge inserted into an analyzer, and determining that the type of cartridge is the test cartridge having a first contact connected to the imager chip and a second contact connected to the electrochemical sensor.

26. The non-transitory machine readable storage medium of claim 20, wherein the plurality of spacer elements have a predetermined average spacer height that defines a predetermined average chamber height of a chamber between the portion of the imager chip and the transparent material layer.

27. The non-transitory machine readable storage medium of claim 20, wherein at least one of the second wall and the plurality of spacer elements is deformable such that the second wall and the plurality of spacer elements are drawn toward each other by capillary force from the blood sample being moved into the sample testing conduit.
